(19) United States
(12) Patent Application Publication
(10) Pub. No.: US 2009/0312245 A1
(43) Pub. Date: Dec. 17, 2009

(54) SRA binding protein

(75) Inventors: Peter Leedman, Perth (AU); Esme Hatchell, Mount Lawley (AU); Shane Colley, West Leederville (AU)

Correspondence Address: Edwards Angell Palmer & Dodge LLP, P.O. Box 55874, Boston, MA, 02205, US

(73) Assignee: The University of Western Australia, Nedlands (AU)

(21) Appl. No.: 12/009,690

(22) Filed: Jan. 22, 2008

Related U.S. Application Data
(63) Continuation of application No. PCT/AU2006/001037, filed on Jul. 24, 2006.

(30) Foreign Application Priority Data
Jul. 22, 2005 (AU) 2005903903
Dec. 23, 2005 (AU) 2005907268
Jun. 7, 2006 (AU) 2006903090

(51) Int. Cl. A61K 38/17; C07K 14/435; C07K 16/18; G01N 33/566; C12N 15/85; C12N 15/12; C12Q 1/68; A01K 67/027; A61P 35/00; A61P 15/00; A61P 3/04; A61P 19/02; A61P 19/10; A61P 9/10

(52) U.S. Cl. 514/12; 530/350; 530/387.9; 436/501; 536/23.4; 435/320.1; 536/23.1; 435/6; 800/13

(57) ABSTRACT

An isolated polypeptide comprising: (i) SEQ ID No: 2; (ii) amino acids 27 to 109 of SEQ ID No: 2 (iii) amino acids 22 to 109 of SEQ ID No: 2 (iv) amino acids 21-91 of SEQ ID No: 2 (v) amino acids 21-26 and/or 60-67 of SEQ ID No: 2 or (vi) a functional variant of any one of (i) to (v).

### RELATED APPLICATIONS

This is a continuation patent application that claims priority to PCT patent application number PCT/AU2006/01037, filed on Jul. 24, 2006, which claims priority to AU patent application number 2006903090 filed on Jun. 7, 2006, which claims priority to AU patent application 2005907268 filed on Dec. 23, 2005 and AU patent application number 2005903903 filed on Jul. 22, 2005, the entirety of which are herein incorporated by reference.

### FIELD OF INVENTION

The present invention relates to SRA binding polypeptides, variants thereof and to related polynucleotides and binding agents. The present invention also relates to various applications of these molecules including methods of treating, diagnosing and preventing serious diseases and disorders such as cancer.

### BACKGROUND TO THE INVENTION

Coregulators, functioning as coactivators or corepressors of nuclear receptor (NR) activity, play pivotal roles in mediating hormone action via the regulation of transcriptional efficiency. The discovery of steroid receptor coactivator-1 (SRC-1), a broad-spectrum coactivator, ten years ago provided important insight into understanding hormone action and the mechanisms underlying transcriptional activation by NRs. Since then, a large family of coregulators has been discovered, each of which is selectively recruited by specific NRs in a ligand- and tissue-specific manner to cognate response elements in the DNA.

For the estrogen receptor (ER), a NR that plays a key role in the proliferation of breast cancer cells, a large number or coregulators have been identified. These include SRC-1, SRC-2/GRIP (glucocorticoid receptor-interacting protein)-1, SRC-3/AIB-1, SRC-2/GRIP (glucocorticoid receptor-interacting protein)-1, SRC-3AIB1 (amplified in breast cancer-1), members of the TRAP (thyroid receptor (TR)-associated proteins/DRIP (vitamin D receptor-interacting proteins) complex, E6-AP (E6-associated protein), MTA1 (metastasis-associated protein 1), MICoA (MTA interacting coactivator), PELP1 (proline-, glutamic acid-, leucine-rich protein 1), SHARP (SMRT/HDAC1 associated repressor protein), DP97 (novel DEAD box helicase 97 kD) and SRA (steroid receptor RNA activator). Remarkably, SRA is the only coregulator that has the capacity to coactivate as an RNA rather than protein, and for this reason stands alone in its functional characteristics.

SRA plays an important role in mediating 17β-estradiol (E₂) action. Its expression is both increased and aberrant in many human breast tumours, suggesting a potential role in pathogenesis. It has been shown that SRA expression was increased in a panel of 26 tumours when compared to normal tissue. Further, an exon 3 deletion of SRA has been described that confers a worse prognosis to specific subsets of breast cancer patients. Despite evidence that an alternative splice variant of SRA may exist as a protein in the rat and possibly in the human, conclusive evidence suggests the recently discovered human SRA is a NR coactivator that can act as an RNA transcript.

Recent findings have identified protein interactors of SRA, and provided insight into the putative mechanisms underlying SRA's transcriptional coactivation ability. Specifically, SHARP a NR corepressor that binds SMRT (silencing mediator for retinoid and thyroid receptors), HDAC (histone deacytlyase)1 and HDAC-2, interacts with SRA in vitro, and contains three RNA recognition motif (RRM) domains. These RRMs are required to repress SRA-augmented E₂-induced transactivation. In pull-down studies, full-length SRA bound to recombinant SHARP, but the detail of where this interaction might occur within the SRA structure has not yet been defined. p72 is another ER coregulator that binds SRA in vitro and co-purifies with full-length SRA from cell extracts. The detail of which substructure of SRA was involved and definitive analysis of the SRA-p72 interaction has also not been reported. Thus, although SRA-protein interactions impact significantly on NR activity and signalling, the specifics of any interaction to date are unclear, and the identity of SRA-binding proteins whose function is dependent upon targeting specific SRA substructures is unknown.

The present invention seeks to identify and characterize a new protein that is capable of interacting with and regulating the action of SRA.

### SUMMARY OF THE INVENTION

The present invention provides an isolated polypeptide comprising:
(i) SEQ ID No: 2;
(ii) amino acids 27 to 109 of SEQ ID No:2;
(iii) amino acids 22 to 109 of SEQ ID No:2;
(iv) amino acids 21 to 91 of SEQ ID No:2;
(v) amino acids 21-26 and/or 54-61 of SEQ ID No:2; or
(vi) a functional variant of any one of (i) to (v).

The SLIRP polypeptides herein such as SEQ ID NO:2 may be used as a biomarker for cancer. The present invention also provides a selective binding agent of any one of (i) to (vi) above such as an antibody or derivative thereof. These may be used in methods of detecting a polypeptide of the invention in a biological sample by a method which comprises:
(i) contacting an antibody to any one of (i) to (vi) above with a biological sample under conditions which allow for the formation of an antibody-antigen complex; and
(ii) determining whether antibody-antigen complex comprising said antibody is formed.

The present invention also provides an isolated polynucleotide encoding a polypeptide of the present invention such as:
(i) SEQ ID No: 2;
(ii) amino acids 27 to 109 of SEQ ID No:2;
(iii) amino acids 22 to 109 of SEQ ID No:2;
(iv) amino acids 21 to 91 of SEQ ID No:2;
(v) amino acids 21-26 and/or 5-61 of SEQ ID No:2; or
(vi) a functional variant of any one of (i) to (v).

The present invention also provides fragments of the polynucleotides herein including fragments that encode polypeptides with at least one important property of the full length polypeptide or epitope bearing portions of the larger polypeptide. Isolated polynucleotides that selectively hybridize with at least a portion of a polynucleotide of the present invention are also provided as part of the present invention.

The present invention also provides a vector comprising an isolated polynucleotide of the present invention and a host cell including such a vector.

The present invention also provides a composition comprising a therapeutically effective amount of a polypeptide or nucleotide described herein in admixture with a pharmaceutically or physiologically acceptable formulation agent selected for suitability with the mode of administration.

An active agent herein such as a polypeptide or selective binding agent can be delivered by implanting certain cells that have been genetically engineered to express and secrete the polypeptide or selective binding agent. Thus, the present invention also provides a method of delivering a therapeutically effective amount of a polypeptide or selective binding agent to a subject using a cell adapted to express a polypeptide according to any one of (i) to (vi) above.

There are a broad range of diseases and disorders that may be treated using therapies based on the polypeptides described herein. Thus, the present invention also provides methods of treating a subject suffering from a disorder associated with undesirable physiological levels of a polypeptide of SEQ ID No:2 comprising the step of manipulating the physiological levels of the polypeptide. Of particular interest are methods of treating cancer and metabolic disorders including those disorders where energy homeostasis is altered, including obesity, insulin resistance and diabetes mellitus (Type II diabetes).

The polypeptide, polynucleotides and binding agents herein may also be used for diagnostic and prognostic purposes in relation to any diseases or disorder associated with undesirable physiological levels of a polypeptide according to SEQ ID No:2.

Thus, the present invention also provides a method for performing a diagnosis on a patient comprising:
(i) determining the concentration of a polypeptide described herein in a biological sample, taken from the patient;
(ii) comparing the level determined in step (i) to the concentration range of the polypeptide known to be present in normal subjects; and
(iii) diagnosing whether the patient has the disorder based on the comparison in step (ii).

The present invention may be applied to assess the prognosis of a patient. Thus, the present invention also provides a method for prognostic evaluation of a patient comprising:
(i) determining the concentration of a polypeptide described herein in a biological sample, taken from the patient;
(ii) comparing the level determined in step (i) to the concentration range of the polypeptide known to be present in normal subjects; and
(iii) evaluating the prognosis of said patient based on the comparison in step (ii).

The present invention also provides for the use of polynucleotides, and more particularly fragments thereof as microarrays. Thus, the present invention also provides a method of determining the expression of a polynucleotide encoding the polypeptide of SEQ ID No:2 comprising the steps of:
(i) extracting mRNA from a sample and converting said mRNA to labelled cDNA;
(ii) contacting the labelled cDNA with an array designed to hybridize to at least one unique sequence within SEQ ID No:1; and
(iii) detecting the bound cDNA.

The present invention also provides a method for detecting the presence or absence of a SLIRP polynucleotide described herein in a biological sample containing nucleic acid which method comprises:
(i) bringing the biological sample into contact with a polynucleotide probe or primer capable of binding to a SLIRP polynucleotide under suitable hybridizing conditions; and
(ii) detecting any duplex formed between the probe or primer and nucleic acid in the sample.

The present invention also provides a method for detecting a SLIRP polypeptide which comprises the sequence set out in SEQ ID No 2 or a homologue, variant, derivative or fragment thereof present in a biological sample which comprises:
(i) incubating a biological sample with a SLIRP antibody described herein under conditions which allow for the formation of an antibody-antigen complex; and
(ii) determining whether an antibody-antigen complex comprising said antibody is formed.

The present invention also includes non-human animals such as mice, rats, or other rodents, rabbits, goats, or sheep, or other farm animals, in which the gene encoding the polypeptide of SEQ ID No:2 or a variant thereof has been disrupted (“knocked out”) such that the level of expression of this gene or genes is(are) significantly decreased or completely abolished.

The present invention also provides for the use of a SLIRP polypeptide or an agonist thereof for repressing the SRA mediated activation of a nuclear receptor.

### BRIEF DESCRIPTION OF THE FIGURES

FIG. 1A: Mfold secondary structure plot of SRA (15). The ΔE value for the full-length SRA structure was −243.1 kJ mol⁻¹. STR7 (labeled) is the most stable and one of the most conserved structures of SRA;

FIG. 1B: RNA electrophoretic mobility shift assay (REMSA) using a ³²P-labeled SRA STR7 probe which was incubated with nuclear and cytoplasmic cell extracts from MCF-7, HeLa and MDA-MB-468 cell lines, before incubation with RNase T1 and heparin followed by 5% PAGE and Phosphorlmager analysis. Some extracts were incubated with excess unlabeled RNA competitor (SRA STR7, pblue vector, tRNA) prior to addition of labeled probe. RPC, RNA-protein complex;

FIG. 1C: UV cross-link assay with nuclear extracts from the cell lines listed in Panel B. Cell extract (30 μg) was incubated with ³²P-labeled STR7 probe, UV irradiated for 10 min, RNase A digested, resolved by SDS-PAGE and detected by PhosphorImager after transfer to PVDF membrane. [¹⁴C]-molecular weight markers were used for size standards. Arrows highlight 39 & 40 kDa RPCs in MDA-MB-468 cells.

FIG. 2A: Nucleotide (nt) and single letter amino acid sequence of SLIRP. The entire mRNA is shown: arrow denotes the sequence isolated via yeast three-hybrid screen; start and stop codons in capitals; italics denote the poly A signal; SLIRP contains a highly conserved RRM (underlined) with consensus RNP2 & RNP1 sub-motifs (highlighted). The symbols *, ̂ and − denote putative N-myristoylation, protein kinase C phosphorylation and casein kinase II phosphorylation sites respectively;

FIG. 2B: Amino acid sequence alignment comparing RRMs of SLIRP, SHARP and nucleolin. Black boxes indicate amino acids conserved with consensus RRM sequence;

FIG. 2C: Schematic of SLIRP and SHARP highlighting specific functional domains. RRM, RNA recognition motif. RID, receptor interaction domain. SID/RD, repression domain. Numbers denote amino acid sequence position;

FIG. 2D: Alignment of human, mouse and rat SLIRP amino acid sequences illustrates high degree of homology between species. Sequences in black indicate amino acid identity, grey, amino acid similarity and white no homology;

FIG. 3A: Northern analysis of SLIRP in normal human tissues. A ³²P-labeled SLIRP cDNA probe was hybridized with a poly A mRNA human tissue blot, before washing and visualization by PhosphorImager. The blots were rehybridized with a β-actin probe for normalization. Small intest., small intestine. Peri. blood leuk., peripheral blood leukocytes;

FIG. 3B: Northern analysis of SLIRP in cancer cell lines. Poly A RNA was generated from a range of human cancer (breast, SK-BR-3, MCF-7, MDA-MB-468; prostate, LNCaP; lung, Calu-6; cervical, HeLa; liver, HepG2), normal mammary (HMEC) and monkey kidney (COS-7) cells, and subjected to Northern analysis with SLIRP, SKIP and GAPDH probes;

FIG. 3C: Protein lysates were prepared from breast (SK-BR-3, MCF-7, MDA-MB-468, T47D), cervical (HeLa), prostate (LNCaP, PC3), lung (Calu-6) and fibrosarcoma (HT1080) cells and transferred to nitrocellulose prior to probing with SLIRP, SKIP or β-actin abs and visualization with ECL;

FIG. 3D: Expression of SLIRP in primary human breast cancer tissue. Sections (20×a,c; 40×b,d) from a human breast ductal cancer were incubated with SLIRP antibody (a, b and d) and compared with sections from the same tumor with no antibody (c). Arrows denote stroma, ducts and tumor tissue. Box in Panel a denotes region magnified in Panel b (40×);

FIG. 4A: HeLa cell extract was incubated with no antibody, SLIRP or β-actin antibody, the resultant immune complexes precipitated with Protein A & G beads (Beads), and copurifying RNA detected by RT-PCR using SRA-specific primers. SRA transcripts were detected in supernatants prior to washing (lanes 1-3) and bead+SLIRP antibody post wash samples (lane 5). No SRA mRNA was detected in samples in the absence of antibody (lane 4), with β-actin antibody (lane 5) or without addition of reverse transcriptase (RT, lanes 7-12). PCR reactions with SRA expression vector (P) or water (W) inputs were included as positive and negative controls, respectively. Arrow denotes the 260 bp SRA-specific PCR product;

FIG. 4B/C: SLIRP knockdown augments SRC-1 association with SRA. MCF-7 cells were treated with siRNA to SLIRP, the cells incubated with no antibody or SRC-1 antibody, and the associated SRA detected as above in (A). Panel B is an RT-PCR for SLIRP, β-actin and SRA that shows SLIRP is significantly knocked down. A small amount of SRA co-purifies with SRC-1 in control cells, but this is substantially augmented in cells with reduced SLIRP. Panel C is an immunoblot showing SLIRP protein is reduced, and that the amount of SRC-1 immunoprecipitated throughout is constant;

FIG. 4D: Schematic of the plasmids used in REMSA studies; GST-alone, GST-SLIRP (wildtype), GST-SHARP-RRM (SHARP aas 1-608) and SHARP-RD (SHARP aas 3420-3651);

FIG. 4E: REMSA using either a ³²P-labeled SRA STR7 or SDM7 mutant probe and recombinant GST-SLIRP shows specificity of binding. Complexes were incubated with RNase T1 and heparin followed by 5% PAGE and PhosphorImager analysis. In lanes 3-4′ and 9-10, excess unlabeled “cold” STR7 competitor RNA (up to 100-fold) was added prior to addition of the labeled wildtype probe. In lanes 5-6, excess cold pblue (vector alone) was added;

FIG. 4F: REMSA using a ³²P-labeled SRA STR7 probe with recombinant GST and GST-SHARP fusion proteins shows binding to SRA using SHARP-RRM (lane 1) but not with either SHARP-RD (lane 2) or GST alone (lane 3);

FIG. 5A: HeLa cells were cotransfected with an ERE-luciferase (Luc) reporter along with expression vectors for ERα±SRA and increasing amounts of SLIRP. After 24 h, transfected cells were treated for a further 8 h with E2 prior to harvesting of lysate and assessment of Luc activity. For some cells, Tam or ICI was added to the well at the same time as E2. Each experiment was performed at least three times in triplicate. Error bars represent standard deviation;

FIG. 5B: HeLa cells were cotransfected with either a GRE-Luc, ARE-Luc, TRE-Luc, VitD-Luc or PPARE-Luc reporter plus corresponding AR, TR, VDR, PPARδ, SRA and SLIRP expression vectors, incubated with ligand (Dex, DHT, T3, VitD, GW501516) for 8 h and Luc activity determined;

FIG. 5C: HeLa cells were cotransfected with GRE-Luc and siRNA directed against either SLIRP or a nonsense target. After 48 h, cells were treated with Dex for 8 h prior to assessment of Luc activity. Reduced endogenous protein expression in SLIRP siRNA treated cells (lane 1) compared with nonsense (lane 2) relative to β-actin, confirmed by Western;

FIG. 5D: HeLa cells were cotransfected with ERE-Luc and expression vectors for ERα alone, and/or empty, SHARP, SLIRP or SKIP vectors, ±siRNA (nonsense, SLIRP or SKIP). RT-PCR confirmed reduced SLIRP and SKIP expression in siRNA treated cells while having no effect on β-actin (lower panel).

FIG. 5E: Schematic of plasmids for expression of wildtype and mutant SLIRP with a carboxy terminal FLAG epitope;

FIG. 5F: REMSA using labeled SRA STR7 probe and increasing amounts of GST-SLIRP fusion proteins (wild-type, mutants R24, 25A, L62A or double mutant R24, 25A, L62A);

FIG. 5G: HeLa cells were transfected as in FIG. 5A with ERE-Luc and either wildtype (SLIRP-FLAG) or mutated SLIRP-FLAG (R24, 25A, L62A, DM) expression vectors together with SRA or SRA SDM7 (a stem-loop mutant);

FIG. 6A: SLIRP is recruited to the endogenous pS2 promoter by estrogen. Chromatin immunoprecipitation (ChIP) assay was employed in MCF-7 cells with ERα antibody as a positive control and HuD antibody, another RRM-containing RNA-binding protein with a nuclear/cytoplasmic distribution, serving as a negative control. Sheared, genomic, MCF-7 DNA was used as input control. SLIRP was recruited to the promoter, whilst HuD was not;

FIG. 6B: Recruitment of SLIRP to the metallothionein (MTA2) promoter is regulated by SRA. HeLa cells were treated with SRA siRNA or non-sense siRNA (NS siRNA) for 3 days, subsequently incubated with Dex and then ChIP assay performed as above with either GR, SRC-1 or SLIRP antibody. The left hand panel shows the ChIP assay, the right hand panel RT-PCR for SRA and actin expression demonstrating the SRA knockdown;

FIG. 6C: MCF-7 cells were treated with either SLIRP siRNA or NS siRNA (3 days) followed by estrogen for 45 min before ChIP assay using ER or NCoR antibody as above. The left hand panel shows the ChIP assay, the right hand panel RT-PCR for SLIRP and actin expression demonstrating the SLIRP knockdown;

FIG. 7A: The mitochondria (red, Mitotracker) and the nuclei (blue, Hoescht 33256) of HeLa cells were simultaneously stained for and the localization of SLIRP detected (green, rabbit polyclonal sera, AlexaFluor 488 secondary antibody). Overlaying of images collected by confocal microscopy reveals colocalization of SLIRP and the mitochondria (yellow) (Top panel). Endogenous SLIRP was also shown to colocalize with HSP-60, a mitochondria specific protein (upper panel). Transfected SLIRP-FLAG colocalized with another mitochondrial protein (cytochrome c) (middle panel), as well as the Mitotracker stain (lower panel). However, transfected FLAG-SLIRP was pan-cellular and did not colocalize with the Mitotracker stain (bottom panel);

FIG. 7B: SLIRP and HSP-60 stain similarly in human breast cancer tissue. IHC of primary human breast cancer tissue using either SLIRP or HSP-60 abs. The duct stained readily with both abs in a punctate cytoplasmic pattern, consistent with a mitochondrial location for HSP-60 and SLIRP;

FIG. 7C: Schematic of SLIRP. Three-dimensional imaging of the SLIRP protein sequence predicts the presence of a mitochondrial localization signal in its amino terminal 26 aas. The positions of aas subjected to point mutation are indicated;

FIG. 7D: Mutations in the SLIRP mitochondrial sequence relieve its repressive activity. HeLa cells were transfected as in FIG. 5A with ERE-luc, ERα, wildtype SRA, and either wildtype SLIRP or the R7, 13, 14A mutant. N=3; error bars represent standard deviation;

FIG. 8A: Immunoblot of variety of lysates from several species including human (HeLa, MCF-7), murine (NIH-3T3, J2E, OD9DL, C2C12), monkey (Cos) and rat brain using the SLIRP polyclonal Antibody or a B-actin Antibody;

FIG. 8B: Immunoblot of GST-fusion proteins used in mutant REMSA binding studies;

FIG. 9: Graph depicting SLIRP mediated regulation of PPARδ signalling activity (with a PPARE-Luc reporter) in the presence of a PPARδ-specific ligand (GW501516) in murine muscle cells;

FIG. 10A: Graph comparing the survival of patients with SLIRP positivity >2+ (on a scoring system of 0-3+, red line) versus those with SLIRP immunoreactivity less than or equal to 2 (blue line).

FIG. 10B: Graph depicts survival with SLIRP staining of >2+ (red line) versus that of women with SLIRP less than or equal to 2 (blue line) specifically within the cohort of women with tumours that were estrogen receptor negative; and

FIG. 11: Figure showing that transfection of SLIRP into human prostate cancer cells (22Rv1 cells) that are androgen responsive, results in a profound reduction of androgen receptor mediated signalling.

### DETAILED DESCRIPTION OF THE INVENTION

Polypeptides

The present invention provides an isolated polypeptide comprising:
(i) SEQ ID No: 2;
(ii) amino acids 27 to 109 of SEQ ID No:2;
(iii) amino acids 22 to 109 of SEQ ID No:2;
(iv) amino acids 21 to 91 of SEQ ID No:2;
(v) amino acids 21-26 and/or 60-67 of SEQ ID No:2; or
(vi) a functional variant of any one of (i) to (v).

For the purposes of the present invention the term “SLIRP polypeptide(s)” and SLIRP, as used herein, includes the above polypeptides, unless the context specifically requires otherwise.

Although not wishing to be bound by any particular mechanism, it is believed that the polypeptides of the present invention modulate (i.e. repress or augment) SRA regulated transactivation of nuclear receptors by binding to SRA at STR7 and affecting the ability of SRA to interact with various other RNA binding proteins that act as co-regulators of nuclear receptor dependent gene expression. It is also believed that the polypeptides herein regulate the capacity of peroxisome proliferator activated receptors (PPARs) to regulate key genes in muscle and thus may have a role in these tissues and more specifically fat metabolism. In addition, the predominant mitochondrial localization of the native polypeptide suggests a role in mitochondrial biogenesis and energy metabolism.

The polypeptides of the present invention may be recombinant, natural or synthetic. The isolated polypeptides of the invention may be mixed with carriers or diluents that will not interfere with the intended purpose of the polypeptide and still be regarded as isolated. A polypeptide of the invention may also be in a substantially purified form, in which case it will generally comprise the polypeptide in a preparation in which at least 90%, 95%, 98% or 99% of the protein in the preparation is a polypeptide of the invention.

It will be recognized that some amino acid sequences of the polypeptides of the invention can be varied without significantly affecting the structure or function of the polypeptide. When such differences in sequence are-contemplated, it should be remembered that there would be critical areas on the protein that determine activity.

Thus, functional variants include isolated polypeptides that have at least one important activity of the polypeptide according to SEQ ID No:2, such as the ability to bind SRA or modulate SRA regulated transactivation of nuclear receptors. Such variants include the recited sequences with deletions, insertions, inversions, repeats, and type substitutions. Guidance concerning which amino acid changes are likely to be phenotypically silent can be found in Bowie, J. U., et al, “Deciphering the Message in Protein Sequences: Tolerance to Amino Acid Substitutions,” Science 247:1306-1310 (1990).

Thus, a variant polypeptide of the present invention may be: (i) one in which one or more of the amino acid residues are substituted with a conserved or non-conserved amino acid residue (preferably a conserved amino acid residue) and such substituted amino acid residue may or may not be one encoded by the genetic code, (ii) one in which one or more of the amino acid residues includes a substituent group, (iii) one in which the polypeptide is fused with another compound, such as a compound to increase the half life of the polypeptide (for example, polyethylene glycol or polypropylene glycol), or (iv) one in which the additional amino acids, such as a leader, signal or secretory sequence or a sequence which is employed for purification of the polypeptide sequence are fused to the mature polypeptide. Such fragments, derivatives and analogs are deemed to be within the scope of those skilled in the art from the teachings herein.

As indicated above, the variants of the present invention may include one or more amino acid substitutions, deletions or additions, either from natural mutations or human manipulation. The particular replacements may be determined by a skilled person as detailed more fully hereunder. However, changes are preferably of a minor nature, such as conservative amino acid substitutions that do not significantly affect the folding or activity of the protein (see for example the table hereunder). Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Amino acids in the polypeptides of the present invention that are essential for function can be identified by methods known in the art, such as site directed mutagenesis or alanine-scanning mutagenesis. The latter procedure introduces single alanine mutations at every residue in the molecule. The resulting mutant molecules are then tested for biological activity such as SRA binding or their ability to modulate SRA regulated transactivation of nuclear receptors. Sites that are critical for ligand-receptor binding can also be determined by structural analysis such as crystallization. Nuclear magnetic resonance or photoaffinity labelling may also be used when developing functional variants. Alternatively, synthetic peptides corresponding to candidate functional variants may be produced and their ability to display one or more activities of the polypeptide of SEQ ID No:2 assessed in vitro or in vivo.

Polypeptide variants of the present invention can also be prepared as libraries using the sequence of SEQ ID No:2 or other polypeptides herein. Phage display can also be effective in identifying variants useful according to the invention. Briefly, a phage library is prepared (using e.g. ml3, fd, or lambda phage), displaying inserts from 4 to about 80 amino acid residues using conventional procedures. The inserts may represent, for example, a biased degenerate array or may completely restrict the amino acids at one or more positions (e.g., for a library based on a protein from SEQ ID No:2). One then can select phage-bearing inserts that have a relevant biological activity of the protein of SEQ ID No:2 such as SRA binding affinity and/or modulation of SRA regulated transactivation of nuclear receptors. This process can be repeated through several cycles of reselection of phage. Repeated rounds lead to enrichment of phage bearing particular sequences. DNA sequence analysis can be conducted to identify the sequences of the expressed polypeptides. The minimal linear portion of the sequence that confers the relevant activity can be determined. One can repeat the procedure using a biased library containing inserts containing part or the entire minimal linear portion plus one or more additional degenerate residues upstream or downstream thereof.

Polypeptides, including variant polypeptides, can be tested for retention of any of the given activity. For example, the peptides can be tested for in vitro properties using transient transfection assays with a responsive reporter that assess the ability of the peptide to modulate SRA regulated transactivation of nuclear receptors to determine which of the variant peptides retain activity.

Preferred variant polypeptides of the present invention comprise an amino acid sequence that is at least 70-80% identical, more preferably at least 90% or 95% identical, still more preferably at least 96%, 97%, 98% or 99% identical to a polypeptide sequence recited herein, such as SEQ ID No:2.

By a polypeptide having an amino acid sequence at least, for example, 95% “identical” to a reference amino acid sequence of a polypeptide of the invention it is intended that the amino acid sequence of the polypeptide is identical to the reference sequence except that the polypeptide sequence may include up to five amino acid alterations per each 100 amino acids of the reference polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular variant polypeptide is at least 90%, 95%, 96%, 97%, 98% or 99% identical to, for instance, SEQ ID No:2 herein can be determined conventionally using known computer programs such the Bestfrt program (Wisconsin Sequence Analysis Package, Version 8 for Unix, Genetics Computer Group, University Research Park, 575 Science Drive, Madison, Wisc. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

In general, the polypeptides of the present invention can be synthesized directly or obtained by chemical or mechanical disruption of larger molecules, fractioned and then tested for one or more activity of the polypeptide of SEQ ID No:2. Functional variants may also be produced by Northern blot analysis of total cellular RNA followed by cloning and sequencing of identified bands derived from different tissues/cells, or by PCR analysis of such RNA also followed by cloning and sequencing. Thus, synthesis or purification of an extremely large number of functional variants is possible using the information contained herein.

Polypeptide variants of the present invention also include fusion proteins, for example, where another peptide sequence is fused to the polypeptide of interest to aid in extraction and purification. Examples of fusion protein partners include glutathione-S-transferase (GST), hexahistidine, GAL4 (DNA binding and/or transcriptional activation domains) and β-galactosidase. It may also be convenient to include a proteolytic cleavage site between the fusion protein partner and the protein sequence of interest to allow removal of fusion protein sequences. Preferably the fusion protein will not hinder SRA binding activity.

The polypeptides of the present invention also include conjugated proteins. In this regard, a protein may be modified by attachment of a moiety (e.g. a fluorescent, radioactive, or enzymatic label, or an unrelated sequence of amino acids to make a fusion protein) that does not correspond to a portion of the peptide in its native state. Thus, the peptides of the present invention may comprise chimeric proteins comprising a fusion of an isolated peptide with another peptide. For example, a peptide capable of targeting the isolated peptide to a cell type or tissue type, enhancing stability of the isolated peptide under assay conditions, or providing a detectable moiety, such as green fluorescent protein. A moiety fused to an isolated peptide or a fragment thereof also may provide means of readily detecting the fusion protein, e.g., by immunological recognition or by fluorescent labelling such as green fluorescent protein. Purified isolated peptides include peptides isolated by methods including, but are not limited to, immunochromotography, HPLC, size-exclusion chromatography, ion-exchange chromatography and immune-affinity chromatography.

The polypeptides herein can be conjugated by well-known methods, including bifunctional linkers, formation of a fusion polypeptide, and formation of biotin/streptavidin or biotin/avidin complexes by attaching either biotin or streptavidin/avidin to the peptide and the complementary molecule. Depending upon the nature of the reactive groups in an isolated peptide and a targeting agent, a conjugate can be formed by simultaneously or sequentially allowing the functional groups of the above-described components to react with one another. Numerous art-recognized methods for forming a covalent linkage can be used. See, e.g., March, J., Advanced Organic Chemistry, 4th Ed., New York, N.Y., Wiley and Sons, 1985), pp. 326-1120.

In general, the conjugated peptides of the invention can be prepared by using well-known methods for forming amide, ester or imino bonds between acid, aldehyde, hydroxy, amino, or hydrazo groups on the respective conjugated peptide components. As would be apparent to one of ordinary skill in the art, reactive functional groups that are present in the amino acid side chains of the peptide preferably are protected, to minimize unwanted side reactions prior to coupling the peptide to the derivatizing agent and/or to the extracellular agent. As used herein, “protecting group” refers to a molecule which is bound to a functional group and which may be selectively removed therefrom to expose the functional group in a reactive form. Preferably, the protecting groups are reversibly attached to the functional groups and can be removed therefrom using, for example, chemical or other cleavage methods. Thus, for example, the peptides of the invention can be synthesized using commercially available side-chain-blocked amino acids (e.g., FMOC-derivatized amino acids from Advanced Chemtech Inc., Louisville, Ky.). Alternatively, the peptide side chains can be reacted with protecting groups after peptide synthesis, but prior to the covalent coupling reaction. In this manner, conjugated peptides of the invention can be prepared in which the amino acid side chains do not participate to any significant extent in the coupling reaction of the peptide to the other agent, such as a cell-type-specific targeting agent.

If a peptide does not have a free amino- or carboxyl-terminal functional group that can participate in a coupling reaction, such a group can be introduced, e.g., by introducing a cysteine (containing a reactive thiol group) into the peptide by synthesis or site directed mutagenesis. Disulfide linkages can be formed between thiol groups in, for example, the peptide and the targeting compound. Alternatively, covalent linkages can be formed using bifunctional cross linking agents, such as bismaleimidohexane (which contains thiol-reactive maleimide groups and which forms covalent bonds with free thiols). See also the Pierce Co. Immunotechnology Catalogue and Handbook Vol. 1 for a list of exemplary homo- and hetero-bifunctional cross linking agents, thiol-containing amines and other molecules with reactive groups.

For peptides that exhibit reduced activity in a conjugated form, the covalent bond between the peptides and its conjugate is selected to be sufficiently labile (e.g., to enzymatic cleavage) so that it is cleaved following transport to its target, thereby releasing the free peptides at the target. Biologically labile covalent linkages, e.g., imino bonds, and “active” esters can be used to form prodrugs where the covalently coupled peptides is found to exhibit reduced activity in comparison to the activity of the peptides alone.

It will be appreciated that the amino acids in the polypeptide of SEQ ID No:2 that are required for activity may be incorporated into larger peptides and still maintain their function. Preferably, the amino acids required for SRA binding include at least amino acids 21 to 26 or 60 to 67 or are other contiguous sequence of between about 5 and 20 amino acids and more preferably between about 6 and 15 amino acids.

Preferably, the isolated peptides are non-hydrolyzable in that the bonds linking the amino acids of the peptide are less readily hydrolyzed than peptide bonds formed between L-amino acids. To provide such peptides, one may select isolated peptides from a library of non-hydrolyzable peptides, such as peptides containing one or more D-amino acids or peptides containing one or more non-hydrolyzable peptide bonds linking amino acids.

Alternatively, one can select peptides that are optimal for a preferred function in suitable assay systems and then modify such peptides as necessary to reduce the potential for hydrolysis by proteases. For example, to determine the susceptibility to proteolytic cleavage, peptides may be labelled and incubated with cell extracts or purified proteases and then isolated to determine which peptide bonds are susceptible to proteolysis, e.g., by sequencing peptides and proteolytic fragments. Alternatively, potentially susceptible peptide bonds can be identified by comparing the amino acid sequence of an isolated peptide with the known cleavage site specificity of a panel of proteases. Based on the results of such assays, individual peptide bonds that are susceptible to proteolysis can be replaced with non-hydrolyzable peptide bonds by in vitro synthesis of the peptide.

Many non-hydrolyzable peptide bonds are known in the art, along with procedures for synthesis of peptides containing such bonds. Non-hydrolyzable bonds include -psi[CH.sub.2 NH]— reduced amide peptide bonds, -psi[COCH.sub.2]- ketomethylene peptide bonds, -psi[CH(CN)NH]— (cyanomethylene)amino peptide bonds, -psi[CH.sub.2 CH(OH)]— hydroxyethylene peptide bonds, -psi[CH.sub.2 O]— peptide bonds, and -psi[CH.sub.2 S]— thiomethylene peptide bonds.

Likewise, various changes may be made including the addition of various side groups that do not affect the manner in which the peptide functions, or which favourably affect the manner in which the peptide functions. Such changes may involve adding or subtracting charge groups, substituting amino acids, adding lipophilic moieties that do not effect binding but that affect the overall charge characteristics of the molecule facilitating a specific outcome with a physiological benefit. For each such change, no more than routine experimentation is required to test whether the molecule functions according to the invention. One simply makes the desired change or selects the desired peptide and applies it in a fashion as described in detail herein.

The peptides herein may also be linked to a variety of polymers, such as polyethylene glycol (PEG) and polypropylene glycol (PPG). Replacement of naturally occurring amino acids with a variety of uncoded or modified amino acids such as D-amino acids and N-methyl amino acids may also be used to modify peptides. Another approach is to use bifunctional crosslinkers, such as N-succinimidyl 3-(2 pyridyldithio)propionate, succinimidyl 6-[3-(2 pyridyldithio)propionamido]hexanoate, and sulfosuccinimidyl 6-[3-2 pyridyidithio)propionamido]hexanoate.

It may be desirable to use derivatives of the peptides of the invention that are conformationally constrained. Conformational constraint refers to the stability and preferred conformation of the three-dimensional shape assumed by a peptide. Conformational constraints include local constraints, involving restricting the conformational mobility of a single residue in a peptide; regional constraints, involving restricting the conformational mobility of a group of residues, which residues may form some secondary structural unit; and global constraints, involving the entire peptide structure.

The active conformation of the peptide may be stabilized by a covalent modification, such as cyclization or by incorporation of gamma-lactam or other types of bridges. For example, side chains can be cyclized to the backbone to create a L-gamma-lactam moiety on each side of the interaction site. Cyclization also can be achieved, for example, by formation of cysteine bridges, coupling of amino and carboxy terminal groups of respective terminal amino acids, or coupling of the amino group of a Lys residue or a related homolog with a carboxy group of Asp, Glu or a related homolog. Coupling of the alpha-amino group of a polypeptide with the epsilon-amino group of a lysine residue, using iodoacetic anhydride, can be also undertaken.

Another approach is to include a metal-ion complexing backbone in the peptide structure. Typically, the preferred metal-peptide backbone is based on the requisite number of particular coordinating groups required by the coordination sphere of a given complexing metal ion. In general, most of the metal ions that may prove useful have a coordination number of four to six. The nature of the coordinating groups in the peptide chain includes nitrogen atoms with amine, amide, imidazole, or guanidino functionalities; sulphur atoms of thiols or disulfides; and oxygen atoms of hydroxy, phenolic, carbonyl, or carboxyl functionalities. In addition, the peptide chain or individual amino acids can be chemically altered to include a coordinating group, such as for example oxime, hydrazino, sulfhydryl, phosphate, cyano, pyridino, piperidino, or morpholino. The peptide construct can be either linear or cyclic, however a linear construct is typically preferred. One example of a small linear peptide is Gly-Gly-Gly-Gly that has four nitrogens (an N₄ complexation system) in the backbone that can complex to a metal ion with a coordination number of four.

Other methods for identifying variants of the isolated peptides herein rely upon the development of amino acid sequence motifs to which potential epitopes may be compared. Each motif describes a finite set of amino acid sequences in which the residues at each (relative) position may be (a) restricted to a single residue, (b) allowed to vary amongst a restricted set of residues, or (c) allowed to vary amongst all possible residues. For example, a motif might specify that the residue at a first position may be any one of valine, leucine, isoleucine, methionine, or phenylalanine; that the residue at the second position must be histidine; that the residue at the third position may be any amino acid residue; that the residue at the fourth position may be any one of the residues valine, leucine, isoleucine, methionine, phenylalanine, tyrosine or tryptophan; that the residue at the fifth position must be lysine, and so on. The motifs in SEQ ID No:2 at amino acids 21-26 and 60-67 provide further assistance to those skilled in the art as search, evaluation, or design criteria for functional variants of the polypeptides disclosed herein.

Thus, the present invention also provides methods for identifying functional variants of an isolated polypeptide. In general, the methods include selecting an isolated peptide, such as the isolated peptide identified herein as SEQ ID No:2.

Then a first amino acid residue of the isolated peptide is mutated to prepare a variant peptide. In one embodiment, the amino acid residue can be selected and mutated as indicated by a computer model of peptide conformation. Peptides bearing mutated residues that maintain a similar conformation (e.g. secondary structure) can be considered potential functional variants that can be tested for function using the assays described herein. Any method for preparing variant peptides can be employed, such as synthesis of the variant peptide, recombinantly producing the variant peptide using a mutated nucleic acid molecule, and the like. The properties of the variant peptide in relation to the isolated peptides described previously are then determined according to standard procedures as described herein.

Variants of the isolated peptides prepared by any of the foregoing methods can be sequenced, if necessary, to determine the amino acid sequence and thus deduce the nucleotide sequence which encodes such variants.

The present invention also provides fragments of the polypeptide of SEQ ID No:2 comprising at least about 10, 20, 30, 50 or 100 amino acid residues. In this context “about” includes the particularly recited range and ranges larger or smaller by several, a few, 5, 4, 3, 2 or 1 amino acid residues at either extreme or at both extremes. For instance, about 40-90 amino acids in this context means a polypeptide fragment of 40 plus or minus several, a few, 5, 4, 3, 2 or 1 amino acid residues to 90 plus or minus several a few, 5, 4, 3, 2 or 1 amino acid residues. Highly preferred in this regard are the recited ranges plus or minus as many as 5 amino acids at either or at both extremes. Particularly highly preferred are the recited ranges plus or minus as many as 3 amino acids at either or at both the recited extremes. Especially particularly highly preferred are ranges plus or minus 1 amino acid at either or at both extremes of the recited ranges with no additions or deletions. Preferably, the fragments include at least one biological activity of the polypeptide from which they are fragmented, such as SRA binding affinity and/or ability to modulate SRA regulated transactivation of nuclear receptors and/or ability to bind an antibody to the full polypeptide.

Fragments or portions of the polypeptides of the present invention may be employed for producing the corresponding full-length polypeptide by peptide synthesis; therefore, the fragments may be employed as intermediates for producing the full-length polypeptides.

Other fragments of the present invention comprise an epitope-bearing portion of a polypeptide according to SEQ ID No:2. Preferably, the epitope is an immunogenic or antigenic epitope of the polypeptide. An “immunogenic epitope” is defined as a part of a protein that elicits an antibody response when the whole protein is the immunogen. On the other hand, a region of a protein molecule to which an antibody can bind is defined as an “antigenic epitope.”

As to the selection of fragments bearing an antigenic epitope (i.e., that contain a region of a protein molecule to which an antibody can bind), it is well known in that art that relatively short synthetic peptides that mimic part of a protein sequence are routinely capable of eliciting an antiserum that reacts with the partially mimicked protein. Peptides capable of eliciting protein-reactive sera are frequently represented in the primary sequence Z-1 of a protein, can be characterized by a set of simple chemical rules, and are confined neither to immunodominant regions of intact proteins (i.e. immunogenic epitopes) nor to the amino or carboxyl terminals. Antigenic epitope-bearing peptides and polypeptides of the invention may be contiguous or conformational epitopes and are useful to raise antibodies, including monoclonal antibodies that bind specifically to a polypeptide of the invention. The epitope-bearing fragments of the invention may be produced by any conventional means apparent to those skilled in the art.

The present invention also includes non-peptide mimetics. A wide variety of techniques may be used to elucidate the precise structure of a peptide. These techniques include amino acid sequencing, x-ray crystallography, mass spectroscopy, nuclear magnetic resonance spectroscopy, computer-assisted molecular modelling, peptide mapping, and combinations thereof. Structural analysis of a peptide provides a large body of data that comprise the amino acid sequence of the peptide as well as the three-dimensional positioning of its atomic components. From this information, non-peptide peptidomimetics may be designed that have the required chemical functionalities for therapeutic activity but are more stable, for example less susceptible to biological degradation.

Thus, variants of the present invention also include mimetics. Nonpeptide analogs of peptides, such as those that provide a stabilized structure or lessened biodegradation, are within the scope of the present invention. Peptide mimetic analogs can be prepared based on a selected peptide by replacement of one or more residues by nonpeptide moieties. Preferably, the nonpeptide moieties permit the peptide to retain its natural conformation, or stabilize a preferred, e.g., bioactive, conformation such as a conformation able to bind SRA. Thus, the present invention also provides for the use of a polypeptide described herein for designing a mimetic thereof such as a non-peptide peptidomimetic.

Selective Binding Agents

As used herein, the term “selective binding agent” refers to a molecule which has specificity for the polypeptides described herein. Suitable selective binding agents include, but are not limited to, antibodies and derivatives thereof, polypeptides and small molecules. Suitable selective binding agents may be prepared using methods known in the art. An exemplary selective binding agent of the present invention is capable of binding a portion of the polypeptides thereby inhibiting or enhancing the binding of the polypeptides to other molecules such as SRA.

Selective binding agents such as antibodies and antibody fragments that bind polypeptides herein, such as SEQ ID No:2, are within the scope of the present invention. The antibodies may be polyclonal including monospecific polyclonal, monoclonal (MAbs), recombinant, chimeric, humanized such as CDR-grafted, human, single chain, and/or bispecific, as well as fragments, variants or derivatives thereof. Antibody fragments include those portions of the antibody that bind to an epitope on the polypeptide. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions.

Polyclonal antibodies generally are produced in animals (e.g., rabbits or mice) by means of multiple subcutaneous or intraperitoneal injections of the polypeptide and an adjuvant. It may be useful to conjugate the polypeptide to a carrier protein that is immunogenic in the species to be immunized, such as keyhole limpet hemocyanin, serum, albumin, bovine thyroglobulin, or soybean trypsin inhibitor. Also, aggregating agents such as alum are used to enhance the immune response. After immunization, the animals are bled and the serum is assayed for antibody titre.

Monoclonal antibodies are produced using any method that provides for the production of antibody molecules by continuous cell lines in culture. Examples of suitable methods for preparing monoclonal antibodies include the hybridoma methods of Kohler et al., Nature, 256:495-497 (1975) and the human B-cell hybridoma method, Kozbor, J. Immunol., 133:3001 (1984);(1984) and Brodeur et al., Monoclonal Antibody Production Techniques and Applications, pp. 51-63 (Marcel Dekker, Inc., New York, 1987). Also provided by the invention are hybridoma cell lines that produce monoclonal antibodies reactive with polypeptides herein.

Monoclonal antibodies of the invention may be modified for use as therapeutics. One embodiment is a “chimeric” antibody in which a portion of the heavy and/or light chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. Also included are fragments of such antibodies, so long as they exhibit the desired biological activity.

In another embodiment, a monoclonal antibody of the invention is a “humanized” antibody. Methods for humanizing non-human antibodies are well known in the art. Generally, a humanized antibody has one or more amino acid residues introduced into it from a source that is non-human. Humanization can be performed, for example, using methods described in the art (Jones et al., Nature 321:522-525 (1986); Riechmann et al., Nature, 332:323-327 (1988); Verhoeyen et al., Science, 239:15341536 (1988)), by substituting at least a portion of a rodent complementarity-determining region (CDR) for the corresponding regions of a human antibody.

Also encompassed by the invention are human antibodies that bind polypeptides herein. Using transgenic animals (e.g., mice) that are capable of producing a repertoire of human antibodies in the absence of endogenous immunoglobulin production, such antibodies are produced by immunization with a polypeptide antigen (i.e., having at least 6 contiguous amino acids), optionally conjugated to a carrier. In one method, such transgenic animals are produced by incapacitating the endogenous loci encoding the heavy and light immunoglobulin chains therein, and inserting loci encoding human heavy and light chain proteins into the genome thereof. Partially modified animals, that is, those having less than the full complement of modifications, are then cross-bred to obtain an animal having all of the desired immune system modifications. When administered an immunogen, these transgenic animals produce antibodies with human (rather than e.g., murine) amino acid sequences, including variable regions that are immunospecific for these antigens. See PCT application nos. PCT/US96/05928 and PCT/US93106926. Additional methods are described in U.S. Pat. No. 5,545,807, PCT application nos. PCT/US91/245, PCT/GB89/01207, and in EP 546073B1 and EP 546073A1. Human antibodies may also be produced by the expression of recombinant DNA in host cells or by expression in hybridoma cells as described herein.

In an alternative embodiment, human antibodies can be produced from phage-display libraries (Hoogenboom et al., J. Mol. Biol., 227:381 (1991); (1991) and Marks et al., J. Mol. Biol., 222:581 (1991)). These processes mimic immune selection through the display of antibody repertoires on the surface of filamentous bacteriophage, and subsequent selection of phage by their binding to an antigen of choice. One such technique is described in PCT Application No. PCT/US98/17364, which describes the isolation of high affinity and functional agonistic antibodies.

Chimeric, CDR grafted, and humanized antibodies are typically produced by recombinant methods. Nucleic acids encoding the antibodies are introduced into host cells and expressed using materials and procedures described herein. In a preferred embodiment, the antibodies are produced in mammalian host cells, such as CHO cells. Monoclonal (e.g., human) antibodies may be produced by the expression of recombinant DNA in host cells or by expression in hybridoma cells as described herein.

The antibodies of the invention may be employed in any known assay method, such as competitive binding assays, direct and indirect sandwich assays, and immunoprecipitation assays (Sola, Monoclonal Antibodies: A Manual of Techniques, pp. 147-158 (CRC Press, Inc., 1987)) for the detection and quantitation of polypeptides. The antibodies will bind polypeptides with an affinity that is appropriate for the assay method being employed.

For diagnostic applications, in certain embodiments, antibodies may be labelled with a detectable moiety. The detectable moiety can be any one that is capable of producing, either directly or indirectly, a detectable signal. For example, the detectable moiety may be a radioisotope, such as ³H, ¹⁴C, ³²P, ³⁵S, or ¹²⁵I, ¹²⁵I; a fluorescent or chemiluminescent compound, such as fluorescein isothiocyanate, rhodamine, or luciferin; or an enzyme, such as alkaline phosphatase, β-galactosidase, or horseradish peroxidase.

Competitive binding assays rely on the ability of a labelled standard (e.g., a polypeptide described herein or an immunologically reactive portion thereof) to compete with the test sample (a candidate polypeptide) for binding with a limited amount of antibody. The amount of the candidate polypeptide in the test sample is inversely proportional to the amount of standard that becomes bound to the antibody. To facilitate determining the amount of standard that becomes bound, the antibodies typically are insolubilized before or after the competition, so that the standard and candidate polypeptide that are bound to the antibodies may conveniently be separated from the standard and candidate polypeptide which remain unbound.

Sandwich assays typically involve the use of two antibodies, each capable of binding to a different immunogenic portion, or epitope, of the protein to be detected and/or quantitated. In a sandwich assay, the test sample (analyte) is typically bound by a first antibody that is immobilized on a solid support, and thereafter a second antibody binds to the analyte, thus forming an insoluble three-part complex. The second antibody may itself be labelled with a detectable moiety (direct sandwich assays) or may be measured using an anti-immunoglobulin antibody that is labelled with a detectable moiety (indirect sandwich assays). For example, one type of sandwich assay is an enzyme-linked immunosorbent assay (ELISA), in which case the detectable moiety is an enzyme.

The selective binding agents, including antibodies, are also useful for in vivo imaging. An antibody labelled with a detectable moiety may be administered to an animal, preferably into the bloodstream, and the presence and location of the labelled antibody in the host is assayed. The antibody may be labelled with any moiety that is detectable in an animal, whether by nuclear magnetic resonance, radiology, or other detection means known in the art.

Selective binding agents of the invention, including antibodies, may be used as therapeutics. These therapeutic agents are generally agonists or antagonists, in that they either enhance or reduce, respectively, at least one of the biological activities of a polypeptide herein, including SRA binding or the ability to modulate SRA regulated transactivation of nuclear receptors. In one embodiment, antagonist antibodies of the invention are antibodies or binding fragments thereof which are capable of specifically binding to a SLIRP polypeptide herein and which are capable of inhibiting or eliminating the functional activity of the polypeptide in vivo or in vitro. In preferred embodiments, the selective binding agent, e.g., an antagonist antibody, will inhibit the functional activity of the polypeptide by at least about 50%, and preferably by at least about 80%. In another embodiment, the selective binding agent may be an antibody that is capable of interacting with SRA or some other binding partner (a ligand or receptor) of the polypeptides described herein thereby inhibiting or eliminating SRA binding activity in vitro or in vivo. Selective binding agents, including agonist and antagonist like antibodies, are identified by screening assays that are well known in the art.

The invention also relates to a kit comprising selective binding agents (such as antibodies) and other reagents useful for detecting the levels of the polypeptides described herein in biological samples. Such reagents may include, a detectable label, blocking serum, positive and negative control samples, and detection reagents.

The polypeptides of the present invention can be used to clone their receptors, using an expression cloning strategy. Radiolabeled (¹²⁵-Iodine) polypeptide or affinity/activity-tagged polypeptide (such as an Fc fusion or an alkaline phosphatase fusion) can be used in binding assays to identify a cell type or cell line or tissue that expresses the receptor(s). RNA isolated from such cells or tissues can be converted to cDNA, cloned into a mammalian expression vector, and transfected into mammalian cells (such as COS or 293 cells) to create an expression library. A radiolabeled or polypeptide of the present invention can then be used as an affinity ligand to identify and isolate from this library the subset of cells which express the receptor(s). DNA can then be isolated from these cells and transfected into mammalian cells to create a secondary expression library in which the fraction of cells expressing receptor(s) is many-fold higher than in the original library. This enrichment process can be repeated iteratively until a single recombinant clone containing the receptor is isolated. Isolation of the receptor(s) is useful for identifying or developing novel agonists and antagonists of the polypeptide signalling pathway. Such agonists and antagonists include soluble polypeptide receptor(s), receptor antibodies, small molecules, proteins, peptides, carbohydrates, lipids, or antisense oligonucleotides, and they may be used for treating, preventing, or diagnosing one or more disease or disorder, including those described herein.

In particular, antibodies may be used to detect polypeptides of the invention present in biological samples by a method that comprises:
(i) providing an antibody of the invention;
(ii) incubating a biological sample with said antibody under conditions which allow for the formation of an antibody-antigen complex; and
(iii) determining whether antibody-antigen complex comprising said antibody is formed.

Suitable samples include extracts of tissues such as brain, skin, breast, ovary, lung, colon, pancreas, testes, liver, muscle, prostate and bone tissues or from neoplastic growths derived from such tissues.

Antibodies of the invention may be bound to a solid support and/or packaged into kits in a suitable container along with suitable reagents, controls, instructions and the like.

SLIRP Binding Protein Assays

One type of assay for identifying substances that bind to the polypeptides of the present invention involves contacting a SLIRP polypeptide described herein, which is immobilized on a solid support, with a non-immobilized candidate substance determining whether and/or to what extent the SLIRP polypeptide and candidate substance bind to each other. Alternatively, the candidate substance may be immobilized and the SLIRP polypeptide non-immobilized.

In a preferred assay method, the SLIRP polypeptide is immobilized on beads such as agarose beads. Typically this is achieved by expressing the component as a GST-fusion protein in bacteria, yeast or higher eukaryotic cell lines and purifying the GST-fusion protein from crude cell extracts using glutathione-agarose beads. As a control, binding of the candidate substance, which is not a GST-fusion protein, to the immobilized SLIRP polypeptide is determined in the absence of the SLIRP polypeptide. The binding of the candidate substance to the immobilized SLIRP polypeptide is then determined. This type of assay is known in the art as a GST pulldown assay. Again, the candidate substance may be immobilized and the SLIRP polypeptide non-immobilized.

It is also possible to perform this type of assay using different affinity purification systems for immobilizing one of the components, for example Ni-NTA agarose and hexahistidine-tagged components.

Binding of the SLIRP polypeptide to the candidate substance may be determined by a variety of methods well-known in the art. For example, the non-immobilized component may be labelled (with for example, a radioactive label, an epitope tag or an enzyme-antibody conjugate). Alternatively, binding may be determined by immunological detection techniques. For example, the reaction mixture can be Western blotted and the blot probed with an antibody that detects the non-immobilized component. ELISA techniques may also be used.

Candidate substances are typically added to a final concentration of from 1 to 1000 nmol/ml, more preferably from 1 to 100 nmol/ml. In the case of antibodies, the final concentration used is typically from 100 to 500 μg/ml, more preferably from 200 to 300 μg/ml.

SLIRP binding proteins can also be identified using the yeast two hybrid method or equivalent to screen libraries using SLIRP as bait. RNA molecules, other than SRA, adapted to bind SLIRP may be identified using SLIRP as bait in a yeast 3 hybrid assay or the clip assay as described by Ule. J. et al (2003) Science 302 1212-1215. Thus, the present invention also provides for the use of SLIRP to identify RNA coregulators.

Another type of in vitro assay involves determining whether a candidate substance modulates binding of a protein/agent known to interact with SLIRP, such as SHARP or SRA. Such an assay typically comprises contacting SLIRP protein with the interacting protein in the presence or absence of the candidate substance and determining if the candidate substance has an effect on SLIRP binding to the interacting protein.

Whole Cell Assays

Candidate substances may also be tested on whole cells for their effect on cell growth. Preferably the candidate substances have been identified by the above-described in vitro methods. Alternatively, rapid throughput screens for substances capable of inhibiting cell growth may be used as a preliminary screen and then used in the in vitro assay described above to confirm that the affect is on SLIRP.

The candidate substance, i.e. the test compound, may be administered to the cell in several ways. For example, it may be added directly to the cell culture medium or injected into the cell. Alternatively, in the case of polypeptide candidate substances, the cell may be transfected with a nucleic acid construct which directs expression of the polypeptide in the cell. Preferably, the expression of the polypeptide is under the control of a regulatable promoter.

Typically, an assay to determine the effect of a candidate substance identified by the method of the invention on cell growth comprises administering the candidate substance to a cell and determining whether the substance affects cell growth. The extent of cell growth may be determined using parameters such as the number and sizes of cell colonies. The extent of growth in treated cells is compared with the extent of growth in an untreated control cell population to determine any affect.

The concentration of candidate substances used will typically be such that the final concentration in the cells is similar to that described above for the in vitro assays.

In a preferred embodiment, the candidate substance is administered to the cell together with functional SLIRP. Since SLIRP can have the effect of reducing cell growth (such as hormone driven cell multiplication/growth), a substance that inhibits SLIRP may serve to restore cell growth back to the levels seen in the absence of SLIRP. Alternatively, if cell growth is further reduced, then the substance may be an activator of SLIRP function or another co-repressor of SRA.

A candidate substance is typically considered to be an inhibitor of SLIRP function if cell growth is increased by at least 10%, preferably at least 20, 30 or 40% relative to the extent of cell growth seen in the presence of SLIRP and absence of the candidate substance. By contrast, a candidate substance is typically considered to be an activator of SLIRP function if cell growth is further decreased by at least 10%, preferably at least 20, 30 or 40% relative to the extent of cell growth seen in the presence of SLIRP and absence of the candidate substance.

Thus, this invention is also particularly useful for screening compounds by using the SLIRP polypeptide or fragment thereof in any of a variety of drug screening techniques.

The SLIRP polypeptide or fragment employed in such a test may either be free in solution, affixed to a solid support, or borne on a cell surface. One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may measure, for example, for the formation of complexes between a SLIRP polypeptide or fragment and the agent being tested, or examine the degree to which the formation of a complex between a SLIRP polypeptide or fragment and a known ligand is interfered with by the agent being tested.

Thus, the present invention provides methods of screening for drugs comprising contacting such an agent with a SLIRP polypeptide or fragment thereof and assaying (i) for the presence of a complex between the agent and the SLIRP polypeptide or fragment, or (ii) for the presence of a complex between the SLIRP polypeptide or fragment and a ligand, by methods well known in the art. In such competitive binding assays the SLIRP polypeptide or fragment is typically labeled. Free SLIRP polypeptide or fragment is separated from that present in a protein:protein complex, and the amount of free (i.e., uncomplexed) label is a measure of the binding of the agent being tested to SLIRP or its interference with SLIRP:ligand binding, respectively.

Another technique for drug screening provides high throughput screening for compounds having suitable binding affinity to the SLIRP polypeptides and is described in detail in Geysen, PCT published application WO 84/03564, published on Sep. 13, 1984. Briefly stated, large numbers of different small peptide test compounds are synthesized on a solid substrate, such as plastic pins or some other surface. The peptide test compounds are reacted with SLIRP polypeptide and washed. Bound SLIRP polypeptide is then detected by methods well known in the art.

Purified SLIRP can be coated directly onto plates for use in the aforementioned drug screening techniques. However, antibodies to the polypeptide can be used to capture antibodies to immobilize the SLIRP polypeptide on the solid phase.

A further technique for drug screening involves the use of host eukaryotic cell lines or cells (such as described above) that have a nonfunctional SLIRP gene. These host cell lines or cells are defective at the SLIRP polypeptide level. The host cell lines or cells are grown in the presence of drug compound. The rate of growth of the host cells is measured to determine if the compound is capable of regulating the growth of SLIRP defective cells.

Polynucleotides

The present invention also provides an isolated polynucleotide encoding a polypeptide of the present invention. Preferably, the polynucleotide encodes the amino acid sequence of an isolated polypeptide comprising:
(i) SEQ ID No: 2;
(ii) amino acids 27 to 109 of SEQ ID No:2;
(iii) amino acids 22 to 109 of SEQ ID No:2;
(iv) amino acids 21 to 91 of SEQ ID No:2;
(v) amino acids 21-26 and/or 60-67 of SEQ ID No:2; or
(vi) a functional variant of any one of (i) to (v).

Polynucleotides of the present invention may be in the form of RNA, such as mRNA, or in the form of DNA, including, for instance, cDNA and genomic DNA obtained by cloning or produced synthetically. In terms of genomic DNA it is noted that the genomic sequence for SLIRP is located between the genomic sequences for alkB and SKIP. The DNA may be double-stranded or single-stranded. Single-stranded DNA or RNA may be the coding strand, also known as the sense strand, or it may be the non-coding strand, also referred to as the anti-sense strand.

Reference to “isolated” polynucleotide(s) means a polynucleotide, DNA or RNA, which has been removed from its native environment. For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution.

Isolated RNA molecules include in vivo or in vitro RNA transcripts of the DNA molecules of the present invention. Isolated polynucleotides according to the present invention further include such molecules produced synthetically.

Polynucleotides of the present invention include those that comprise a nucleotide sequence different to those specifically described herein but which, due to the degeneracy of the genetic code, still encode the same polypeptide. Of course, the genetic code is well known in the art. Thus, it would be routine for one skilled in the art to generate such degenerate variants of the polynucleotides of the present invention such as SEQ ID No:1.

The present invention also provides fragments of the polynucleotides of the present invention. Preferred fragments comprise at least 10, 20, 30, 40, 50, 60 or 70 contiguous nucleotides. Other preferred fragments encode polypeptides with at least one important property of the full length polypeptide or epitope bearing portions of the larger polypeptide. Methods for determining fragments would be readily apparent to one skilled in the art and are exemplified in more detail below.

The polynucleotides of the present invention may be used in accordance with the present invention for a variety of applications, particularly those that make use of the chemical and biological properties of the polypeptide of SEQ ID No:2.

The present invention also provides isolated polynucleotides that selectively hybridize with at least a portion of a polynucleotide of the present invention. As used herein to describe nucleic acids, the term “selectively hybridize” excludes the occasional randomly hybridizing nucleic acids under at least moderate stringency conditions. Thus, selectively hybridizing polynucleotides preferably hybridize under at least moderate stringency conditions and more preferably under high stringency conditions. The hybridizing polynucleotides may be used, for example, as probes or primers for detecting the presence of polynucleotides encoding polypeptides such as SEQ ID No:2 e.g. cDNA or mRNA.

A nucleic acid molecule is “hybridizable” to another nucleic acid molecule, such as a cDNA, genomic DNA, or RNA, when a single-stranded form of the nucleic acid molecule can anneal to the other nucleic acid molecule under the appropriate conditions of temperature and solution ionic strength. The conditions of temperature and ionic strength determine the “stringency” of the hybridization. For preliminary screening for homologous nucleic acids, low stringency hybridization conditions, corresponding to a Tₘ of 55° C., can be used, e.g., 5×SSC, 0.1% SDS, 0.25% milk, and no formamide; or 30% formamide, 5×SSC, 0.5% SDS). Moderate stringency hybridization conditons correspond to a higher Tₘ, e.g., 40% formamide, with 5× or 6×SCC. High stringency hybridization conditions correspond to the highest Tₘ, e.g., 50% formnamide, 5× or 6×SCC.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order RNA:RNA, DNA:RNA, DNA:DNA. For hybrids of greater than 100 nucleotides in length, equations for calculating Tₘ have been derived and are known to those skilled in the art. For hybridization with shorter nucleic acids, i.e., oligonucleotides, the position of mismatches becomes more important, and the length of the oligonucleotide determines its specificity. Preferably a minimum length for a hybridizable nucleic acid is at least about 10 nucleotides; more preferably at least about 15 nucleotides; most preferably the length is at least about 20, 30 or 40-70 nucleotides.

Of course, a polynucleotide which hybridizes only to a poly A sequence (such as a 3′ terminal poly(A) tail of a polynucleotide of the present invention), or to a complementary stretch of T (or U) residues, would not be included as a selectively hybridizable polynucleotide of the invention, since such a polynucleotide would hybridize to any nucleic acid molecule containing a poly (A) stretch or the complement thereof (e.g., practically any double-stranded cDNA clone).

Using the nucleic acid sequences taught herein and relying on cross-hybridization, one skilled in the art can identify polynucleotides in other species that encode polypeptides of the invention. If used as primers, the invention provides compositions including at least two nucleic acids that selectively hybridize with different regions of the target nucleic acid so as to amplify a desired region. Depending on the length of the probe or primer, the target region can range between 70% complementary bases and full complementarity.

The selectively hybridizable polynucleotides described herein or more particularly portions thereof can be used to detect the nucleic acid of the present invention in samples by methods such as the polymerase chain reaction, ligase chain reaction, hybridization, and the like. Alternatively, these sequences can be utilized to produce an antigenic protein or protein portion, or an active protein or protein portion.

In addition, portions of the selectively hybridizable polynucleotides described herein can be selected to selectively hybridize with homologous polynucleotides in other organisms. These selectively hybridizable polynucleotides can be used, for example, to simultaneously detect related sequences for cloning of homologues of the polynucleotides of the present invention.

As indicated above, the polynucleotides of the present invention that encode a polypeptide of the present invention include, but are not limited to, those encoding the amino acid sequence of the polypeptide of SEQ ID No:2, by itself. Rather the polynucleotides of the present invention may comprise the coding sequence for the polypeptide and additional sequences, such as those encoding a leader or secretory sequence, such as a pre-, or pro- or prepro-protein sequence; the coding sequence of the polypeptide, with or without the aforementioned additional coding sequences, together with additional, non-coding sequences, including for example, but not limited to introns and non-coding 5′ and 3′ sequences, such as the transcribed, non-translated sequences that play a role in transcription, mRNA processing, including splicing and polyadenylation signals, for example ribosome binding and stability of mRNA; an additional coding sequence which codes for additional amino acids, such as those which provide additional functionalities. Polynucleotides according to the present invention also include those encoding a polypeptide, such as the entire protein, lacking the N terminal methionine.

Thus, polynucleotides of the present invention include those with a sequence encoding a polypeptide of the invention fused to a marker sequence, such as a sequence encoding a peptide that facilitates purification of the fused polypeptide.

In certain preferred embodiments of this aspect of the invention, the marker amino acid sequence is a hexa histidine peptide, such as the tag provided in a pQE vector (Qiagen, Inc.), among others, many of which are commercially available. The “HA” tag is another peptide useful for purification which corresponds to an epitope derived from the influenza hemagglutinin protein.

The present invention further relates to variants of the nucleic acid molecules of the present invention, which encode variants of the polypeptides of the present invention. Variants may occur naturally, such as a natural allelic variant. By an “allelic variant” is intended one of several alternate forms of a gene occupying a given locus on a chromosome of an organism. Non-naturally occurring variants may be produced using mutagenesis techniques known to those in the art.

Such variants include those produced by nucleotide substitutions, deletions or additions that may involve one or more nucleotides. The variants may be altered in coding regions, non-coding regions, or both. Alterations in the coding regions may produce conservative or non-conservative amino acid substitutions, deletions or additions. Especially preferred among these are silent substitutions, additions and deletions, which do not alter the properties and activities of the encoded polypeptide. Also especially preferred in this regard are conservative substitutions.

The present invention also includes isolated polynucleotides comprising a nucleotide sequence at least 60, 70, 80 or 90% identical, and more preferably at least 95%, 96%, 97%, 98% or 99% identical to a nucleotide sequence encoding the polypeptide having the complete amino acid sequence in SEQ ID NO: 2.

For the purposes of the present invention a nucleotide sequence that is 95% identical to a reference sequence is identical to the reference sequence except that it may include up to five point mutations per each 100 nucleotides of the reference nucleotide sequence. In other words, to obtain a polynucleotide having a nucleotide sequence at least 95% identical to reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 60, 70, 80, 90%, 95%, 96%, 97%, 98% or 99% 90%, 95%, 96%, 97%, 98% or 99% identical to, for instance, the nucleotide sequence encoding a polypeptide according to SEQ ID No:2 can be determined conventionally using known computer programs such as the Bestfit program (Wisconsin Sequence Analysis Package, Version 8 for Unix, Genetics Computer Group, University Research Park, 575 Science Drive, Madison, Wisc. 53711). Bestfit uses the local homology algorithm of Smith and Waterman, Advances in Applied Mathematics 2: 482-489 (1981), to find the best segment of homology between two sequences. When using Bestfit or any other sequence alignment program to determine whether a particular sequence is, for instance, 95% identical to a reference sequence according .to the present invention, the parameters are set, of course, such that the percentage of identity is calculated over the full length of the reference nucleotide sequence and that gaps in homology of up to 5% of the total number of nucleotides in the reference sequence are allowed.

Of course, due to the degeneracy of the genetic code, one of ordinary skill in the art will immediately recognize that a large number of the nucleic acid molecules having a sequence at least 60, 70, 80, 90, 95, 96, 97, 98 or 99 identical to the nucleic acid sequence of the polypeptides in SEQ ID No:2 will encode a polypeptide within the scope of the present invention. In fact, since degenerate variants of these nucleotide sequences all encode the same polypeptide, this will be clear to the skilled artisan even without performing the above described comparison.

It will be further recognized in the art that, for such nucleic acid molecules that are not degenerate variants, a reasonable number will also encode a polypeptide having one or more properties of the full polypeptide such as SRA binding and or the ability to repress SRA mediated activation of nuclear receptors. This is because the skilled artisan is fully aware of amino acid substitutions that are either less likely or not likely to significantly effect protein function (e.g., replacing one aliphatic amino acid with a second aliphatic amino acid).

Screening Methods

The polynucleotides herein may be used to, screen for mutations in a gene encoding a polypeptide according to the present invention and/or to secure expression of the polypeptides described herein with SRA binding activity or another biological activity of SEQ ID No:2.

A polynucleotide is said to “encode” a polypeptide if, in its native state or when manipulated by methods well known to those skilled in the art, it can be transcribed and/or translated to produce the mRNA for and/or the polypeptide or a fragment thereof. The anti-sense strand is the complement of such a nucleic acid, and the encoding sequence can be deduced therefrom.

cDNA or genomic libraries of various types may be screened as natural sources of the polynucleotides of the present invention, or such polynucleotides may be provided by amplification of sequences resident in genomic DNA or other natural sources, e.g., by PCR. The choice of cDNA libraries normally corresponds to a tissue source that is abundant in mRNA for the desired proteins. Phage libraries are normally preferred, but other types of libraries may be used. Clones of a library are spread onto plates, transferred to a substrate for screening, denatured and probed for the presence of desired sequences.

The nucleic acid sequences used in this invention will usually comprise at least about five codons (15 nucleotides), more usually at least about 7-15 codons, and most preferably, at least about 35 codons. One or more introns may also be present. This number of nucleotides is usually about the minimal length required for a successful probe that would hybridize specifically with a polynucleotide sequence of interest.

Techniques for nucleic acid manipulation are described generally, for example, in Sambrook et al., 1989: “Molecular Cloning: a laboratory manual. Sambrook, J., Fritsch, E. F. and Maniatis, T. (1989). Coldspring Harbour Laboratory Press, Coldspring Harbour, N.Y. or Ausubel et al., 1992 Current Protocols in Molecular Biology. Ausubel, F. M., Brent, R., Kingston, R. E., Moore, D. D., Seidman, J. G., Smith, J. G. and Struhl, K. (1987). John Wiley and Sons, NY. Reagents useful in applying such techniques, such as restriction enzymes and the like, are widely known in the art and commercially available from such vendors as New England BioLabs, Boehringer Mannheim, Amersham, Promega Biotec, U.S. Biochemicals, New England Nuclear, and a number of other sources. The recombinant nucleic acid sequences used to produce fusion proteins of the present invention may be derived from natural or synthetic sequences. Many natural gene sequences are obtainable from various cDNA or from genomic libraries using appropriate probes. See, GenBank, National Institutes of Health.

Probe sequences may also hybridize specifically to duplex DNA under certain conditions to form triplex or other higher order DNA complexes. The preparation of such probes and suitable hybridization conditions are well known in the art.

Detectably labeled nucleic acid molecules hybridizable to a DNA molecule of the invention are also provided and include nucleic acid molecules hybridizable to a non-coding region of an nucleic acid encoding a polypeptide of the present invention, which non-coding region is selected from the group consisting of an intron, a 5′ non-coding region, and a 3′ non-coding region. The present invention also provides oligonucleotide primers for amplifying human genomic DNA encoding a polypeptide described herein such as oligonucleotides set out in the Examples.

Polynucleotide polymorphisms associated with alleles of the polynucleotide of SEQ ID No:1 which predispose to certain disorders such as cancer or are associated with disorders such as cancer can be detected by hybridization with a polynucleotide probe which forms a stable hybrid with that of the target sequence, under stringent to moderately stringent hybridization and wash conditions. If it is expected that the probes will be perfectly complementary to the target sequence, stringent conditions will be used. Hybridization stringency may be lessened if some mismatching is expected, for example, if variants are expected with the result that the probe will not be completely complementary. Conditions are chosen which rule out nonspecific/adventitious bindings, that is, which minimize noise. Since such indications identify neutral DNA polymorphisms as well as mutations, these indications need further analysis to demonstrate detection of a disease susceptible allele.

Probes for the alleles may be derived from the sequences of SEQ ID No: 1 or its corresponding gene. The probes may be of any suitable length, which span all or a portion of the gene or SEQ ID No:1, and which allow specific hybridization to a region of interest. If the target sequence contains a sequence identical to that of the probe, the probes may be short, e.g., in the range of about 8-30 base pairs, since the hybrid will be relatively stable under even stringent conditions. If some degree of mismatch is expected with the probe, i.e., if it is suspected that the probe will hybridize to a variant region, a longer probe may be employed which hybridizes to the target sequence with the requisite specificity.

The probes include an isolated polynucleotide attached to a label or reporter molecule and may be used to isolate other polynucleotide sequences, having sequence similarity by standard methods. For techniques for preparing and labeling probes see, e.g. Sambrook et al., 1989: “Molecular Cloning: a laboratory manual. Sambrook, J., Fritsch, E. F. and Maniatis, T. (1989). Coldspring Harbour Laboratory Press, Coldspring Harbour, N.Y. or Ausubel et al., 1992 Current Protocols in Molecular Biology. Ausubel, F. M., Brent, R., Kingston, R. E., Moore, D. D., Seidman, J. G., Smith, J. G. and Struhl, K. (1987). John Wiley and Sons, NY. Other similar polynucleotides may be selected by using homologous polynucleotides. Alternatively, polynucleotides encoding these or similar polypeptides may be synthesized or selected by use of the redundancy in the genetic code. Various codon substitutions may be introduced, e.g., by silent changes (thereby producing various restriction sites) or to optimize expression for a particular system. Mutations may be introduced to modify the properties of the polypeptide, perhaps to change ligand-binding affinities, interchain affinities, or the polypeptide degradation or turnover rate.

Probes comprising synthetic oligonucleotides or other polynucleotides of the present invention may be derived from naturally occurring or recombinant single- or double-stranded polynucleotides, or be chemically synthesized. Probes may also be labeled by nick translation, Klenow fill-in reaction, or other methods known in the art.

Portions of the polynucleotide sequence having at least about eight nucleotides, usually at least about 15 nucleotides, and fewer than about 6 kb, usually fewer than about 1.0 kb, from a polynucleotide sequence encoding a polypeptide according to the present invention or fragment thereof are preferred as probes. The probes may also be used to determine whether mRNA encoding the polypeptide is present in a cell or tissue and whether the genomic organization of the genes locus is deleted or otherwise damaged.

A variety of DNA technologies may thus be used to identify mutant alleles in a range of individuals. A number of these alleles may comprise minor alterations to the genomic sequence, such as point mutations including insertions deletions and/or substitutions. Fragments of nucleic acid which comprise these mutations may be used in diagnostic screening as described below. Accordingly, the present invention provides one or more polynucleotides or fragments thereof as described herein comprising mutations with respect to the wild type sequence. In a further embodiment, the present invention provides a plurality of polynucleotides or fragments thereof as described herein for use in screening the DNA of an individual for the presence of one or more mutations/polymorphisms. The plurality of sequences is conveniently provided immobilized to a solid substrate as is described below.

Nucleic Acid Arrays

Polynucleotides of the invention, including probes that may be used to detect both normal (wild type) and abnormal SLIRP sequences in nucleic acid samples, may be immobilized to a solid phase support. The probes will typically form part of a library of DNA molecules that may be used to detect simultaneously a number of different genes in a given genome.

Techniques for producing immobilized libraries of DNA molecules have been described in the art. Generally, most prior art methods describe the synthesis of single-stranded nucleic acid molecule libraries, using for example masking techniques to build up various permutations of sequences at the various discrete positions on the solid substrate. U.S. Pat. No. 5,837,832, the contents of which are incorporated herein by reference, describes an improved method for producing DNA arrays immobilized to silicon substrates based on very large scale integration technology. In particular, U.S. Pat. No. 5,837,832 describes a strategy called “tiling” to synthesize specific sets of probes at spatially-defined locations on a substrate which may be used to produce the immobilized DNA libraries of the present invention. U.S. Pat. No. 5,837,832 also provides references for earlier techniques that may also be used. Thus, nucleic acid probes may be synthesized in situ on the surface of the substrate.

Alternatively, single-stranded molecules may be synthesized off the solid substrate and each pre-formed sequence applied to a discrete position on the solid substrate. For example, nucleic acids may be printed directly onto the substrate using robotic devices equipped with either pins or pizo electric devices.

The library sequences are typically immobilized onto or in discrete regions of a solid substrate. The substrate may be porous to allow immobilization within the substrate or substantially non-porous, in which case the library sequences are typically immobilized on the surface of the substrate. The solid substrate may be made of any material to which polypeptides can bind, either directly or indirectly. Examples of suitable solid substrates include flat glass, silicon wafers, mica, ceramics and organic polymers such as plastics, including polystyrene and polymethacrylate. It may also be possible to use semi-permeable membranes such as nitrocellulose or nylon membranes, which are widely available. The semi-permeable membranes may be mounted on a more robust solid surface such as glass. The surfaces may optionally be coated with a layer of metal, such as gold, platinum or other transition metal. A particular example of a suitable solid substrate is the commercially available BiaCore™ chip (Pharmacia Biosensors).

Preferably, the solid substrate is generally a material having a rigid or semi-rigid surface. In preferred embodiments, at least one surface of the substrate will be substantially flat, although in some embodiments it may be desirable to physically separate synthesis regions for different polymers with, for example, raised regions or etched trenches. It is also preferred that the solid substrate is suitable for the high density application of DNA sequences in discrete areas of typically from 50 to 100 μm, giving a density of 10000 to 40000 cm⁻².

The solid substrate is conveniently divided up into sections. This may be achieved by techniques such as photoetching, or by the application of hydrophobic inks, for example teflon-based inks (Cel-line, USA).

Discrete positions, in which each different member of the library is located may have any convenient shape, e.g., circular, rectangular, elliptical, wedge-shaped, etc.

Attachment of the nucleic acid sequences to the substrate may be by covalent or non-covalent means. The nucleic acid sequences may be attached to the substrate via a layer of molecules to which the library sequences bind. For example, the nucleic acid sequences may be labelled with biotin and the substrate coated with avidin and/or streptavidin. A convenient feature of using biotinylated nucleic acid sequences is that the efficiency of coupling to the solid substrate can be determined easily. Since the nucleic acid sequences may bind only poorly to some solid substrates, it is often necessary to provide a chemical interface between the solid substrate (such as in the case of glass) and the nucleic acid sequences. Examples of suitable chemical interfaces include hexaethylene glycol. Another example is the use of polylysine coated glass, the polylysine then being chemically modified using standard procedures to introduce an affinity ligand. Other methods for attaching molecules to the surfaces of solid substrate by the use of coupling agents are known in the art, see for example WO98149557.

Binding of complementary nucleic acid sequence to the immobilized nucleic acid library may be determined by a variety of means such as changes in the optical characteristics of the bound nucleic acid (i.e. by the use of ethidium bromide) or by the use of labelled nucleic acids, such as polypeptides labelled with fluorophores. Other detection techniques that do not require the use of labels include optical techniques such as optoacoustics, reflectometry, ellipsometry and surface plasmon resonance (SPR)—see WO97/49989, incorporated herein by reference.

Thus, the present invention provides a solid substrate having immobilized thereon at least one polynucleotide of the present invention, preferably two or more different polynucleotides of the present invention, for example two or more different polynucleotides corresponding to different alleles. In a preferred embodiment the solid substrate further comprises polynucleotides derived from genes other than the SLIRP gene.

High throughput expression profiling has a broad range of applications with respect to the polypeptides of the invention, including, but not limited to: the identification and validation of disease-related genes as targets for therapeutics; molecular toxicology of polypeptides of the invention and inhibitors thereof; stratification of populations and generation of surrogate markers for clinical trials; and enhancing polypeptide related small molecule drug discovery by aiding in the identification of selective compounds in high throughput screens (HTS).

Vectors and Host Cells

A nucleic acid molecule encoding the amino acid sequence of a polypeptide herein may be inserted into an appropriate expression vector using standard ligation techniques. The vector is typically selected to be functional in the particular host cell employed (i.e., the vector is compatible with the host cell machinery such that amplification of the gene and/or expression of the gene can occur). A nucleic acid molecule encoding the amino acid sequence of polypeptide herein may be amplified/expressed in prokaryotic, yeast, insect (baculovirus systems), and/or eukaryotic host cells. Selection of the host cell will depend in part on whether the polypeptide is to be post-translationally modified (e.g., glycosylated and/or phosphorylated). If so, yeast, insect, or mammalian host cells are preferable.

Typically, expression vectors used in any of the host cells will contain sequences for plasmid maintenance and for cloning and expression of exogenous nucleotide sequences. Such sequences, collectively referred to as “flanking sequences” in certain embodiments, will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Each of these sequences is discussed below.

Optionally, the vector may contain a “tag”-encoding sequence, i.e., an oligonucleotide molecule located at the 5′ or 3′ end of the polypeptide coding sequence; the oligonucleotide sequence encodes polyHis (such as hexaHis), or another “tag” such as FLAG, HA (hemaglutinin influenza virus) or myc for which commercially available antibodies exist. This tag is typically fused to the polypeptide upon expression of the polypeptide, and can serve as a means for affinity purification of the polypeptide from the host cell. Affinity purification can be accomplished, for example, by column chromatography using antibodies against the tag as an affinity matrix. Optionally, the tag can subsequently be removed from the purified polypeptide by various means such as using certain peptidases for cleavage.

Flanking sequences may be homologous (i.e., from the same species and/or strain as the host cell), heterologous (i.e., from a species other than the host cell species or strain), hybrid (i.e., a combination of flanking sequences from more than one source) or synthetic, or the flanking sequences may be native sequences that normally function to regulate polypeptide expression. As such, the source of a flanking sequence may be any prokaryotic or eukaryotic organism, any vertebrate or invertebrate organism, or any plant, provided that the flanking sequence is functional in, and can be activated by, the host cell machinery.

The flanking sequences useful in the vectors of this invention may be obtained by any of several methods well known in the art. Typically, flanking sequences useful herein other than the gene flanking sequences will have been previously identified by mapping and/or by restriction endonuclease digestion and can thus be isolated from the proper tissue source using the appropriate restriction endonucleases. In some cases, the full nucleotide sequence of a flanking sequence may be known. Here, the flanking sequence may be synthesized using the methods described herein for nucleic acid synthesis or cloning.

Where all or only a portion of the flanking sequence is known, it may be obtained using PCR and/or by screening a genomic library with suitable oligonucleotide and/or flanking sequence fragments from the same or another species. Where the flanking sequence is not known, a fragment of DNA containing a flanking sequence may be isolated from a larger piece of DNA that may contain, for example, a coding sequence or even another gene or genes. Isolation may be accomplished by restriction endonuclease digestion to produce the proper DNA fragment followed by isolation using agarose gel purification, Qiagen® column chromatography (Chatsworth, Calif.), or other methods known to the skilled artisan. The selection of suitable enzymes to accomplish this purpose will be readily apparent to one of ordinary skill in the art.

An origin of replication is typically a part of those prokaryotic expression vectors purchased commercially, and the origin aids in the amplification of the vector in a host cell. Amplification of the vector to a certain copy number can, in some cases, be important for the optimal expression of the polypeptide. If the vector of choice does not contain an origin of replication site, one may be chemically synthesized based on a known sequence, and ligated into the vector. For example, the origin of replication from the plasmid pBR322 (Product No. 303-3s, New England Biolabs, Beverly, Mass.) is suitable for most Gram-negative bacteria, and various origins (e.g., SV40, polyoma, adenovirus, vesicular stomatitus virus (VSV) or papillomaviruses such as HPV or BPV) are useful for cloning vectors in mammalian cells. Generally, the origin of replication component is not needed for mammalian expression vectors (for example, the SV40 origin is often used only because it contains the early promoter).

A transcription termination sequence is typically located 3′ of the end of a polypeptide coding region and serves to terminate transcription. Usually, a transcription termination sequence in prokaryotic cells is a G-C rich fragment followed by a poly T sequence. While the sequence is easily cloned from a library or even purchased commercially as part of a vector, it can also be readily synthesized using methods for nucleic acid synthesis such as those described herein.

A selectable marker gene element encodes a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells, (b) complement auxotrophic deficiencies of the cell; or (c) supply critical nutrients not available from complex media. Preferred selectable markers are the kanamycin resistance gene, the ampicillin resistance gene, and the tetracycline resistance gene. A neomycin resistance gene may also be used for selection in prokaryotic and eukaryotic host cells.

Other selection genes may be used to amplify the gene that will be expressed. Amplification is the process wherein genes which are in greater demand for the production of a protein critical for growth are reiterated in tandem within the chromosomes of successive generations of recombinant cells. Examples of suitable selectable markers for mammalian cells include dihydrofolate reductase (DHFR) and thymidine kinase. The mammalian cell transformants are placed under selection pressure which only the transformants are uniquely adapted to survive by virtue of the selection gene present in the vector. Selection pressure is imposed by culturing the transformed cells under conditions in which the concentration of selection agent in the medium is successively changed, thereby leading to the amplification of both the selection gene and the DNA that encodes a polypeptide described herein. As a result, increased quantities of the polypeptide are synthesized from the amplified DNA.

A ribosome binding site is usually necessary for translation initiation of mRNA and is characterized by a Shine-Dalgarno sequence (prokaryotes) or a Kozak sequence (eukaryotes). The element is typically located 3′ to the promoter and 5′ to the coding sequence of the polypeptide to be expressed. The Shine-Dalgarno sequence is varied but is typically a polypurine (i.e., having a high A-G content). Many Shine-Dalgarno sequences have been identified, each of which can be readily synthesized using methods set forth herein and used in a prokaryotic vector.

A leader, or signal, sequence may be used to direct the polypeptide out of the host cell. Typically, a nucleotide sequence encoding the signal sequence is positioned in the coding region of the nucleic acid molecule encoding the polypeptide, or directly at the 5′ end of the polypeptide coding region. Many signal sequences have been identified, and any of those that are functional in the selected host cell may be used in conjunction with the nucleic acid molecule. Therefore, a signal sequence may be homologous (naturally occurring) or heterologous to the gene or cDNA encoding the polypeptide. Additionally, a signal sequence may be chemically synthesized using methods described herein. In most cases, the secretion of the polypeptide from the host cell via the presence of a signal peptide will result in the removal of the signal peptide from the secreted polypeptide. The signal sequence may be a component of the vector, or it may be a part of the nucleic acid molecule that is inserted into the vector.

Included within the scope of this invention is the use of either a nucleotide sequence encoding a native signal sequence joined to a polypeptide coding region or a nucleotide sequence encoding a heterologous signal sequence joined to a polypeptide coding region. The heterologous signal sequence selected should be one that is recognized and processed, i.e., cleaved by a signal peptidase, by the host cell. For prokaryotic host cells that do not recognize and process the native polypeptide signal sequence, the signal sequence is substituted by a prokaryotic signal sequence selected, for example, from the group of the alkaline phosphatase, penicillinase, or heat-stable enterotoxin II leaders. For yeast secretion, the native polypeptide signal sequence may be substituted by the yeast invertase, alpha factor, or acid phosphatase leaders. In mammalian cell expression the native signal sequence is satisfactory, although other mammalian signal sequences may be suitable.

In some cases, such as where glycosylation is desired in a eukaryotic host cell expression system, one may manipulate the various presequences to improve glycosylation or yield. For example, one may alter the peptidase cleavage site of a particular signal peptide, or add presequences, which also may affect glycosylation. The final protein product may have, in the −1 position (relative to the first amino acid of the mature protein), one or more additional amino acids incidental to expression, which may not have been totally removed. For example, the final protein product may have one or two amino acid residues found in the peptidase cleavage site, attached to the N-terminus. Alternatively, use of some enzyme cleavage sites may result in a slightly truncated form of the desired polypeptide, if the enzyme cuts at such area within the mature polypeptide.

In many cases, transcription of a nucleic acid molecule is increased by the presence of one or more introns in the vector; this is particularly true where a polypeptide is produced in eukaryotic host cells, especially mammalian host cells. The introns used may be naturally occurring within the gene, especially where the gene used is a full length genomic sequence or a fragment thereof. Where the intron is not naturally occurring within the gene (as for most cDNAs), the intron(s) may be obtained from another source. The position of the intron with respect to flanking sequences and the gene is generally important, as the intron must be transcribed to be effective. Thus, when a cDNA molecule is being transcribed, the preferred position for the intron is 3′ to the transcription start site, and 5′ to the polyA transcription termination sequence. Preferably, the intron or introns will be located on one side or the other (i.e., 5′ or 3′) of the cDNA such that it does not interrupt the coding sequence. Any intron from any source, including any viral, prokaryotic and eukaryotic (plant or animal) organisms, may be used to practice this invention, provided that it is compatible with the host cell(s) into which it is inserted. Also included herein are synthetic introns. Optionally, more than one intron may be used in the vector.

The expression and cloning vectors of the present invention will each typically contain a promoter that is recognized by the host organism and operably linked to the molecule encoding the polypeptide. Promoters are untranscribed sequences located upstream (5′) to the start codon of a structural gene (generally within about 100 to 1000 bp) that control the transcription of the structural gene. Promoters are conventionally grouped into one of two classes, inducible promoters and constitutive promoters. Inducible promoters initiate increased levels of transcription from DNA under their control in response to some change in culture conditions, such as the presence or absence of a nutrient or a change in temperature. Constitutive promoters, on the other hand, initiate continual gene product production; that is, there is little or no control over gene expression. A large number of promoters, recognized by a variety of potential host cells, are well known. A suitable promoter is operably linked to the DNA encoding the polypeptide by removing the promoter from the source DNA by restriction enzyme digestion and inserting the desired promoter sequence into the vector. The native gene promoter sequence may be used to direct amplification and/or expression of a nucleic acid molecule. A heterologous promoter is preferred, if it permits greater transcription and higher yields of the expressed protein as compared to the native promoter, and if it is compatible with the host cell system that has been selected for use.

Promoters suitable for use with prokaryotic hosts include the beta-lactamase and lactose promoter systems; alkaline phosphatase, a tryptophan (trp) promoter system; and hybrid promoters such as the tac promoter. Other known bacterial promoters are also suitable. Their sequences have been published, thereby enabling one skilled in the art to ligate them to the desired DNA sequence(s), using linkers or adapters as needed to supply any useful restriction sites.

Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowl pox virus, adenovirus (such as Adenovirus 2), bovine papilloma virus, avian sarcoma virus, cytomegalovirus (CMV), a retrovirus, hepatitis-B virus and most preferably Simian Virus 40 (SV40). Other suitable mammalian promoters include heterologous mammalian promoters, e.g., heat-shock promoters and the actin promoter.

Additional promoters which may be of interest in controlling gene transcription include, but are not limited to: the SV40 early promoter region; the CMV promoter, the promoter contained in the 3′ long terminal repeat of Rous sarcoma virus; the herpes thymidine kinase promoter, the regulatory sequences of the metallothionine gene, prokaryotic expression vectors such as the beta-lactamase promoter; or the tac promoter. Also of interest are the following animal transcriptional control regions, which exhibit tissue specificity and have been utilized in transgenic animals: the elastase I gene control region which is active in pancreatic acinar cells; the insulin gene control region which is active in pancreatic beta cells; the immunoglobulin gene control region which is active in lymphoid cells; the mouse mammary tumor virus control region which is active in testicular, breast, lymphoid and mast cells; the albumin gene control region which is active in liver; the alphafetoprotein gene control region which is active in liver, the alpha 1-antitrypsin gene control region which is active in the liver; the beta-globin gene control region which is active in myeloid cells; the myelin basic protein gene control region which is active in oligodendrocyte cells in the brain; the myosin light chain-2 gene control region which is active in skeletal muscle; and the gonadotropic releasing hormone gene control region which is active in the hypothalamus.

An enhancer sequence may be inserted into the vector to increase the transcription of a DNA encoding the polypeptide of the present invention by higher eukaryotes. Enhancers are cis-acting elements of DNA, usually about 10-300 bp in length, that act on the promoter to increase transcription. Enhancers are relatively orientation and position independent. They have been found 5′ and 3′ to the transcription unit. Several enhancer sequences available from mammalian genes are known (e.g., globin, elastase, albumin, alpha-feto-protein and insulin). Typically, however, an enhancer from a virus will be used. The SV40 enhancer, the cytomegalovirus early promoter enhancer, the polyoma enhancer, and adenovirus enhancers are exemplary enhancing elements for the activation of eukaryotic promoters. While an enhancer may be spliced into the vector at a position 5′ or 3′ to a nucleic acid molecule, it is typically located at a site 5′ from the promoter.

Expression vectors of the invention may be constructed from a starting vector such as a commercially available vector. Such vectors may or may not contain all of the desired flanking sequences. Where one or more of the desired flanking sequences are not already present in the vector, they may be individually obtained and ligated into the vector. Methods used for obtaining each of the flanking sequences are well known to one skilled in the art.

Preferred vectors for practicing this invention are those that are compatible with bacterial, insect, and mammalian host cells. Such vectors include, inter alia, pCRII, pCR3, and pcDNA3.1 (Invitrogen Company, Carlsbad, Calif.), pBSII (Stratagene Company, La Jolla, Calif.), pET15□ (Novagen, Madison, Wisc.), pGEX (Pharmacia Biotech, Piscataway, N.J.), pEGFP-N2 (Clontech, Palo Alto, Calif.), pETL (BlueBacII; Invitrogen), pDSR-alpha (PCT Publication No. WO 90/14363) and pFastBacDual (Gibco/BRL, Grand Island, N.Y.).

Additional suitable vectors include, but are not limited to, cosmids, plasmids or modified viruses, but it will be appreciated that the vector system must be compatible with the selected host cell. Such vectors include, but are not limited to, plasmids such as Bluescript® plasmid derivatives (a high copy number ColE1-based phagemid, Stratagene Cloning Systems Inc., La Jolla Calif.), PCR cloning plasmids designed for cloning Taq-Taq-amplified PCR products (e.g., TOPO™ TA Cloning® Kit, PCR2.1® plasmid derivatives, Invitrogen, Carlsbad, Calif.), and mammalian, yeast, or virus vectors such as a baculovirus expression system (pBacPAK plasmid derivatives, Clontech, Palo Alto, Calif.).

After the vector has been constructed and a nucleic acid molecule encoding the polypeptide has been inserted into the proper site of the vector, the completed vector may be inserted into a suitable host cell for amplification and/or polypeptide expression. The transformation of an expression vector for a polypeptide into a selected host cell may be accomplished by well known methods including transfection, infection, calcium chloride, electroporation, microinjection, lipofection or the DEAE-dextran method or other known techniques. The method selected will in part be a function of the type of host cell to be used. These methods and other suitable methods are well known to the skilled artisan, and are set forth, for example, in Sambrook et al., supra.

Host cells may be prokaryotic host cells (such as E. coli) or eukaryotic host cells (such as a yeast cell, an insect cell or a vertebrate cell). The host cell, when cultured under appropriate conditions, synthesizes the polypeptide that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity, such activity (such as glycosylation or phosphorylation), and ease of folding into a biologically active molecule.

A number of suitable host cells are known in the art and many are available from the American Type Culture Collection (ATCC), 10801 University Boulevard, Manassas, Va. 20110-2209. Examples include, but are not limited to, mammalian cells, such as Chinese hamster ovary cells (CHO) (ATCC No. CCL61); CHO DHFR-cells (Urlaub et al., Proc. Nat. Acad. Sci. USA, 97:4216-4220 (1980)); human embryonic kidney (HEK) 293 or 293T cells (ATCC No. CRL1573); or 3T3 cells (ATCC No. CCL92). The selection of suitable mammalian host cells and methods for transformation, culture, amplification, screening and screening, product production and purification are known in the art. Other suitable mammalian cell lines, are the monkey COS-1 (ATCC No. CRL1650) and COS-7cell lines (ATCC No. CRL1651) cell lines, and the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants, are also suitable. Candidate cells may be genotypically deficient in the selection gene, or may contain a dominantly acting selection gene. Other suitable mammalian cell lines include, but are not limited to, mouse neuroblastoma N2A cells, HeLa, mouse L-L-929 cells, 3T3 lines derived from Swiss, Balb-c or NIH mice, BHK or HaK hamster cell lines, which are available from the ATCC. Each of these cell lines is known by and available to those skilled in the art of protein expression.

Similarly useful as host cells suitable for the present invention are bacterial cells. For example, the various strains of E. coli (e.g., HB101, (ATCC No. 33694) DH5α, DH10, and MC1061 (ATCC No. 53338)) are well-well known as host cells in the field of biotechnology. Various strains of B. subtillis, Pseudomonas spp., other Bacillus spp., Streptomyces spp., and the like may also be employed in this method.

Many strains of yeast cells known to those skilled in the art are also available as host cells for the expression of the polypeptides of the present invention. Preferred yeast cells include, for example, Saccharomyces cerivisae and Pichia pastors.

Additionally, where desired, insect cell systems may be utilized in the methods of the present invention. Such systems are described for example in Kitts et al, Biotechniques, 14:810-817 (1993); Lucklow, Curr. Opin. Biotechnol., 4:564-572 (1993); and Lucklow et al. (J. al., J. Virol., 67:4566-4579 (1993). Preferred insect cells are Sf-9 and Hi5 (Invitrogen, Carlsbad, Calif.).

One may also use transgenic animals to express glycosylated polypeptides of the present invention. For example, one may use a transgenic milk-producing animal (a cow or goat, for example) and obtain the present glycosylated polypeptide in the animal milk. One may also use plants to produce polypeptides. However, in general, the glycosylation occurring in plants is different from that produced in mammalian cells, and may result in a glycosylated product which is not suitable for human therapeutic use.

Pharmaceutical Compositions

Therapeutic compositions are within the scope of the present invention. Such compositions may comprise a therapeutically effective amount of a polypeptide or nucleotide described herein in admixture with a pharmaceutically or physiologically acceptable formulation agent selected for suitability with the mode of administration. Pharmaceutical compositions may also comprise a therapeutically effective amount of one or more selective binding agents described herein in admixture with a pharmaceutically or physiologically acceptable formulation agent selected for suitability with the mode of administration.

The pharmaceutical composition may contain formulation materials for modifying, maintaining or preserving, for example, the pH, osmolarity, viscosity, clarity, colour, isotonicity, odour, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin), fillers; monosaccharides, disaccharides; and other carbohydrates (such as glucose, mannose, or dextrins); proteins (such as serum albumin, gelatin or immunoglobulins); colouring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapol); stability enhancing agents (sucrose or sorbitol); tonicity enhancing agents (such as alkali metal halides, preferably sodium or potassium chloride), delivery vehicles, diluents, excipients and/or pharmaceutical adjuvants.

The optimal pharmaceutical composition will be determined by one skilled in the art depending upon, for example, the intended route of administration, delivery format, and desired dosage. Such compositions may influence the physical state, stability, rate of in vivo release, and rate of in vivo clearance of the active agent.

The primary vehicle or carrier in a pharmaceutical composition may be either aqueous or non-aqueous in nature. For example, a suitable vehicle or carrier may be water for injection, physiological saline solution, solution or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute therefor. In one embodiment of the present invention, pharmaceutical compositions may be prepared for storage by mixing the selected composition having the desired degree of purity with optional formulation agents in the form of a lyophilized cake or an aqueous solution. Further, the polypeptide product may be formulated as a lyophilizate using appropriate excipients such as sucrose.

The pharmaceutical compositions can be capable of parenteral delivery. Alternatively, the compositions may be capable of inhalation or for delivery through the digestive tract, such as orally. The preparation of such pharmaceutically acceptable compositions is within the skill of the art.

The formulation components are present in concentrations that are acceptable to the site of administration. For example, buffers are used to maintain the composition at physiological pH or at a slightly lower pH, typically within a pH range of from about 5 to about 8.

When parenteral administration is contemplated, the therapeutic compositions for use in this invention may be in the form of a pyrogen-free, parenterally acceptable aqueous solution comprising the desired polypeptide or nucleotide in a pharmaceutically acceptable vehicle. A particularly suitable vehicle for parenteral injection is sterile distilled water in which the active agent is formulated as a sterile, isotonic solution, properly preserved. Yet another preparation can involve the formulation of the desired molecule with an agent, such as injectable microspheres, bio-erodible particles, polymeric compounds (such as polylactic acid, acid or polyglycolic acid), or beads or liposomes, that provides for the controlled or sustained release of the product which may then be delivered as a depot injection. Hyaluronic acid may also be used, and this may have the effect of promoting sustained duration in the circulation. Other suitable means for the introduction of the desired molecule include implantable drug delivery devices.

In one embodiment, a pharmaceutical composition may be formulated for inhalation. For example, a polypeptide or nucleotide may be formulated as a dry powder for inhalation. The polypeptide or nucleic acid molecule inhalation solutions may also be formulated with a propellant for aerosol delivery. In yet another embodiment, solutions may be nebulized. Pulmonary administration is further described in PCT application no. PCT/US94/001875, which describes pulmonary delivery of chemically modified proteins.

It is also contemplated that certain formulations may be administered orally. In one embodiment of the present invention, polypeptide or nucleotides of the present invention that are administered in this fashion can be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents can be included to facilitate absorption of the active agent. Diluents, flavourings, low melting point waxes, vegetable oils, lubricants, suspending agents, tablet disintegrating agents, and binders may also be employed.

Another pharmaceutical composition may involve an effective quantity of the polypeptide or nucleotide in a mixture with non-toxic excipients that are suitable for the manufacture of tablets. By dissolving the tablets in sterile water, or another appropriate vehicle, solutions can be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving polypeptides in sustained- or controlled-delivery formulations. Techniques for formulating a variety of other sustained- or controlled-delivery means, such as liposome carriers, bio-erodible microparticles or porous beads and depot injections, are also known to those skilled in the art. See for example, PCT Application No. PCT/US93/00829 that describes the controlled release of porous polymeric microparticles for the delivery of pharmaceutical compositions. Additional examples of sustained-sustained-release preparations include semipermeable polymer matrices in the form of shaped articles, e.g. films, or microcapsules. Sustained release matrices may include polyesters, hydrogels, polylactides, copolymers of L-glutamic acid and gamma ethyl-L-glutamate, ethylene vinyl acetate or poly-D(−)-3-hydroxybutyric acid. Sustained-release compositions may also include liposomes, which can be prepared by any of several methods known in the art.

The pharmaceutical composition to be used for in vivo administration typically must be sterile. This may be accomplished by filtration through sterile filtration membranes. Where the composition is lyophilized, sterilization using these methods may be conducted either prior to, or following, lyophilization and reconstitution. The composition for parenteral administration may be stored in lyophilized form or in a solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or as a dehydrated or lyophilized powder. Such formulations may be stored either in a ready-to-use form or in a form (e.g., lyophilized) requiring reconstitution prior to administration.

The effective amount of the active agent in the pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the active agent is being used, the route of administration, and the size (body weight, body surface or organ size) and condition (the age and general health) of the patient. Accordingly, the clinician may titre the dosage and modify the route of administration to obtain the optimal therapeutic effect. A typical dosage may range from about 0.1 μg/kg to up to about 100 mg/kg or more, depending on the factors mentioned above. In other embodiments, the dosage may range from 0.1 μg/kg up to about 100 mg/kg; or 1 μg/kg up to about 100 mg/kg; or 5 μg/kg up to about 100 mg/kg.

The frequency of dosing will depend upon the pharmacokinetic parameters of the active agent and the formulation used. Typically, a clinician will administer the composition until a dosage is reached that achieves the desired effect. The composition may therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data.

The route of administration of the pharmaceutical composition is in accord with known methods, e.g., orally, through injection by intravenous, intraperitoneal, intracerebral (intra-parenchymal), intracerebroventricular, intramuscular, intra-ocular, intraarterial, intraportal, or intralesional routes; by sustained release systems or by implants. Where desired, the compositions may be administered by bolus injection or continuously by infusion, or by implantation device.

Alternatively or additionally, the composition may be administered locally via implantation of a membrane, sponge or another appropriate material on to which the desired molecule has been absorbed or encapsulated. Where an implantation device is used, the device may be implanted into any suitable tissue or organ, and delivery of the desired molecule may be via diffusion, timed-release bolus, or continuous administration.

In some cases, it may be desirable to use the pharmaceutical compositions herein in an ex vivo manner. In such instances, cells, tissues, or organs that have been removed from the patient are exposed to the pharmaceutical compositions after which the cells, tissues and/or organs are subsequently implanted back into the patient.

Gene/Cell Therapy

An active agent herein such as a polypeptide or selective binding agent can be delivered by implanting certain cells that have been genetically engineered, using methods such as those described herein, to express and secrete the polypeptide or selective binding agent. Such cells may be animal or human cells, and may be autologous, heterologous, or xenogenic. Optionally, the cells may be immortalized. In order to decrease the chance of an immunological response, the cells may be encapsulated to avoid infiltration of surrounding tissues. The encapsulation materials are typically biocompatible, semi-permeable polymeric enclosures or membranes that allow the release of the protein product(s) but prevent the destruction of the cells by the patient's immune system or by other detrimental factors from the surrounding tissues.

Additional embodiments of the present invention relate to cells and methods (e.g., homologous recombination and/or other recombinant production methods) for both the in vitro production of therapeutic polypeptides and for the production and delivery of therapeutic polypeptides by gene therapy or cell therapy. Homologous and other recombination methods may be used to modify a cell that contains a normally transcriptionally silent transcriptionally silent gene encoding a polypeptide described herein, or an under expressed gene, and thereby produce a cell which expresses therapeutically efficacious amounts of the polypeptides.

Homologous recombination is a technique originally developed for targeting genes to induce or correct mutations in transcriptionally active genes. The basic technique was developed as a method for introducing specific mutations into specific regions of the mammalian genome or to correct specific mutations within defective genes. Through homologous recombination, a given DNA sequence to be inserted into the genome can be directed to a specific region of the gene of interest by attaching it to targeting DNA. The targeting DNA is a nucleotide sequence that is complementary (homologous) to a region of the genomic DNA. Small pieces of targeting DNA that are complementary to a specific region of the genome are put in contact with the parental strand during the DNA replication process.

It is a general property of DNA that has been inserted into a cell to hybridize, and therefore, recombine with other pieces of endogenous DNA through shared homologous regions. If this complementary strand is attached to an oligonucleotide that contains a mutation or a different sequence or an additional nucleotide, it too is incorporated into the newly synthesized strand as a result of the recombination. As a result of the proofreading function, it is possible for the new sequence of DNA to serve as the template. Thus, the transferred DNA is incorporated into the genome. Attached to these pieces of targeting DNA are regions of DNA that may interact with or control the expression of a polypeptide herein, e.g., flanking sequences. For example, a promoter/enhancer element, a suppressor or an exogenous transcription modulatory element is inserted in the genome of the intended host cell in proximity and orientation sufficient to influence the transcription of DNA encoding the desired polypeptide. The control element controls a portion of the DNA present in the host cell genome. Thus, the expression of the desired polypeptide of the present invention may be achieved not by transfection of DNA that encodes the polypeptide itself, but rather by the use of targeting DNA (containing regions of homology with the endogenous gene of interest), coupled with DNA regulatory segments that provide the endogenous gene sequence with recognizable signals for transcription of the gene encoding the polypeptide.

In an exemplary method, the expression of a desired targeted gene in a cell (i.e., a desired endogenous cellular gene) is altered via homologous recombination into the cellular genome at a preselected site, by the introduction of DNA that includes at least a regulatory sequence, an exon and a splice donor site. These components are introduced into the chromosomal (genomic) DNA in such a manner that this, in effect, results in the production of a new transcription unit (in which the regulatory sequence, the exon and the splice donor site present in the DNA construct are operatively linked to the endogenous gene). As a result of the introduction of these components into the chromosomal DNA, the expression of the desired endogenous gene is altered.

Altered gene expression, as described herein, encompasses activating (or causing to be expressed) a gene which is normally silent (unexpressed) in the cell as obtained, as well as increasing the expression of a gene which is not expressed at physiologically significant levels in the cell as obtained. The embodiments further encompass changing the pattern of regulation or induction such that it is different from the pattern of regulation or induction that occurs in the cell as obtained, and reducing (including eliminating) the expression of a gene which is expressed in the cell as obtained.

One method by which homologous recombination can be used to increase, or cause production of a polypeptide described herein from a cell's endogenous gene involves first using homologous recombination to place a recombination sequence from a site-specific recombination system (e.g., Cre/IoxP, FLP/FRT) (see, Sauer, Current Opinion In Biotechnology, 5:521-527, 1994; and Sauer, Methods In Enzymology, 225:890-900, 1993) upstream (that is, 5′ to) of the cell's endogenous genomic polypeptide coding region. A plasmid containing a recombination site homologous to the site that was placed just upstream of the genomic polypeptide coding region is introduced into the modified cell line along with the appropriate recombinase enzyme. This recombinase enzyme causes the plasmid to integrate, via the plasmid's recombination site, into the recombination site located just upstream of the genomic polypeptide coding region in the cell line (Baubonis and Sauer, Nucleic Acids Res., 21:2025-2029, 1993; and O'Gorman et al., Science, 251: 1351-1355, 1991). Any flanking sequences known to increase transcription (e.g., enhancer/promoter, intron or translational enhancer), if properly positioned in this plasmid, would integrate in such a manner as to create a new or modified transcriptional unit resulting in de novo or increased polypeptide production from the cell's endogenous gene.

A further method to use the cell line in which the site-specific recombination sequence has been placed just upstream of the cell's endogenous genomic polypeptide coding region is to use homologous recombination to introduce a second recombination site elsewhere in the cell line's genome. The appropriate recombinase enzyme is then introduced into the two-recombination-site cell line, causing a recombination event (deletion, inversion or translocation) (Sauer, Current Opinion In Biotechnology, supra, 1994 and Sauer, Methods In Enzymology, supra, 1993) that would create a new or modified transcriptional unit resulting in de novo or increased polypeptide production from the cell's endogenous gene.

Another approach for increasing, or causing, the expression of the polypeptide from a cell's endogenous gene involves increasing, or causing, the expression of a gene or genes (e.g., transcription factors) and/or decreasing the expression of a gene or genes (e.g., transcriptional repressors) in a manner which results in de novo or increased polypeptide production from the cell's endogenous gene. This method includes the introduction of a non-naturally occurring polypeptide (e.g., a polypeptide comprising a site-specific DNA binding domain fused to a transcriptional factor domain) into the cell such that de novo or increased polypeptide production from the cell's endogenous gene results.

The present invention further relates to DNA constructs useful in the method of altering expression of a target gene. In certain embodiments, the exemplary DNA constructs comprise: (a) one or more targeting sequences; (b) a regulatory sequence; (c) an exon; and (d) an unpaired splice-donor site. The targeting sequence in the DNA construct directs the integration of elements (a)-(d) into a target gene in a cell such that the elements (b)-(d) are operatively linked to sequences of the endogenous target gene. In another embodiment, the DNA constructs comprise: (a) one or more targeting sequences, (b) a regulatory sequence, (c) an exon, (d) a splice-donor site, (e) an intron, and (f) a splice-acceptor site, wherein the targeting sequence directs the integration of elements (a)-(f) such that the elements of (b)-(f) are operatively linked to the endogenous gene. The targeting sequence is homologous to the preselected site in the cellular chromosomal DNA with which homologous recombination is to occur. In the construct, the exon is generally 3′ of the regulatory sequence and the splice-donor site is 3′ of the exon.

If the sequence of a particular gene is known, such as the nucleic acid sequence of the polypeptides presented herein, a piece of DNA that is complementary to a selected region of the gene can be synthesized or otherwise obtained, such as by appropriate restriction of the native DNA at specific recognition sites bounding the region of interest. This piece serves as a targeting sequence(s) upon insertion into the cell and will hybridize to its homologous region within the genome. If this hybridization occurs during DNA replication, this piece of DNA, and any additional sequence attached thereto, will act as an Okazaki fragment and will be incorporated into the newly synthesized daughter strand of DNA. The present invention, therefore, includes nucleotides encoding a polypeptide, which nucleotides may be used as targeting sequences.

Polypeptide cell therapy, e.g., the implantation of cells producing polypeptides described herein, is also contemplated. This embodiment involves implanting cells capable of synthesizing and secreting a biologically active form of the polypeptide. Such polypeptide-producing cells can be cells that are natural producers of the polypeptides or may be recombinant cells whose ability to produce the polypeptides has been augmented by transformation with a gene encoding the desired polypeptide or with a gene augmenting the expression of the polypeptide. Such a modification may be accomplished by means of a vector suitable for delivering the gene as well as promoting its expression and secretion. In order to minimize a potential immunological reaction in patients being administered a polypeptide, as may occur with the administration of a polypeptide of a foreign species, it is preferred that the natural cells producing polypeptide be of human origin and produce human polypeptide. Likewise, it is preferred that the recombinant cells producing polypeptide be transformed with an expression vector containing a gene encoding a human polypeptide.

Implanted cells may be encapsulated to avoid the infiltration of surrounding tissue. Human or non-human animal cells may be implanted in patients in biocompatible, semipermeable polymeric enclosures or in membranes that allow the release of polypeptide, but prevent the destruction of the cells by the patient's immune system or by other detrimental factors from the surrounding tissue. Alternatively, the patient's own cells, transformed to produce polypeptides ex Vivo, may be implanted directly into the patient without such encapsulation.

Techniques for the encapsulation of living cells are known in the art, and the preparation of the encapsulated cells and their implantation in patients may be routinely accomplished. For example, Baetge et al. (WO 95/05452 and PCT/US94/09299) describe membrane capsules containing genetically engineered cells for the effective delivery of biologically active molecules. The capsules are biocompatible and are easily retrievable. The capsules encapsulate cells transfected with recombinant DNA molecules comprising DNA sequences coding for biologically active molecules operatively linked to promoters that are not subject to down-regulation in vivo upon implantation into a mammalian host. The devices provide for the delivery of the molecules from living cells to specific sites within a recipient. A system for encapsulating living cells is described in PCT Application no. PCT/US91/00157 of Aebischer et al. See also, PCT Application no. PCT/US91/00155 of Aebischer et al.; Winn et al., Exper. Neurol., 113:322-329 (1991), Aebischer et al., Exper. Neurol., 11 1:269-275 (1991); and Tresco et al., ASAIO, 38:17-23 (1992).

In vivo and in vitro gene therapy delivery of polypeptides are also part of the present invention. One example of a gene therapy technique is to use the gene (either genomic DNA, cDNA, and/or synthetic DNA) encoding a polypeptide described herein that may be operably linked to a constitutive or inducible promoter to form a “gene therapy DNA construct”. The promoter may be homologous or heterologous to the endogenous gene, provided that it is active in the cell or tissue type into which the construct will be inserted. Other components of the gene therapy DNA construct may optionally include, DNA molecules designed for site-specific integration (e.g., endogenous sequences useful for homologous recombination); tissue-specific promoter, enhancer(s) or silencer(s); DNA molecules capable of providing a selective advantage over the parent cell; DNA molecules useful as labels to identify transformed cells; negative selection systems, cell specific systems; cell-specific binding agents (as, for example, for cell targeting); cell-specific internalization factors; and transcription factors to enhance expression by a vector, as well as factors to enable vector manufacture.

A gene therapy DNA construct can then be introduced into cells (either ex vivo or in vivo) using viral or non-viral vectors. Certain vectors, such as retroviral vectors, will deliver the DNA construct to the chromosomal DNA of the cells, and the gene can integrate into the chromosomal DNA. Other vectors will function as episomes, and the gene therapy DNA construct will remain in the cytoplasm.

In yet other embodiments, regulatory elements can be included for the controlled expression of the gene in the target cell. Such elements are turned on in response to an appropriate effector. In this way, a therapeutic polypeptide can be expressed when desired. One conventional control means involves the use of small molecule dimerizers or rapalogs (as described in WO 9641865 (PCT/US96/099486); WO 9731898 (PCT/US97/03137) and WO9731899 (PCT/US95/03157) used to dimerize chimeric proteins which contain a small molecule-binding domain and a domain capable of initiating biological process, such as a DNA-binding protein or a transcriptional activation protein. The dimerization of the proteins can be used to initiate transcription of the transgene.

An alternative regulation technology uses a method of storing proteins expressed from the gene of interest inside the cell as an aggregate or cluster. The gene of interest is expressed as a fusion protein that includes a conditional aggregation domain that results in the retention of the aggregated protein in the endoplasmic reticulum. The stored proteins are stable and inactive inside the cell. The proteins can be released, however, by administering a drug (e.g., small molecule ligand) that removes the conditional aggregation domain and thereby specifically breaks apart the aggregates or clusters so that the proteins may be secreted from the cell.

Another control means uses a positive tetracycline-controllable transactivator. This system involves a mutated tet repressor protein DNA-binding domain (mutated tet R-4 amino acid changes which resulted in a reverse tetracycline-regulated transactivator protein, i.e., it binds to a tet operator in the presence of tetracycline) linked to a polypeptide that activates transcription.

In vivo gene therapy may be accomplished by introducing the gene encoding a polypeptide into cells via local injection of a nucleic acid molecule or by other appropriate viral or non-non-viral delivery vectors. For example, a nucleic acid molecule encoding a polypeptide of the present invention may be contained in an adeno-associated virus (AAV) vector for delivery to the targeted cells (e.g., Johnson, International Publication No. WO95/34670; and International Application No. PCT/US95/07178). The recombinant AAV genome typically contains AAV inverted terminal repeats flanking a DNA sequence encoding a polypeptide operably linked to functional promoter and polyadenylation sequences.

Alternative suitable viral vectors include, but are not limited to, retrovirus, adenovirus, herpes simplex virus, lentivirus, hepatitis virus, parvovirus, papovavirus, poxvirus, alphavirus, coronavirus, rhabdovirus, paramyxovirus, and papilloma virus vectors. U.S. Pat. No. 5,672,344 describes an in vivo viral-mediated gene transfer system involving a recombinant neurotrophic HSV-1 vector. U.S. Pat. No. 5,399,346 provides examples of a process for providing a patient with a therapeutic protein by the delivery of human cells that have been treated in vitro to insert a DNA segment encoding a therapeutic protein. Additional methods and materials for the practice of gene therapy techniques are described in U.S. Pat. No. 5,631,236 involving adenoviral vectors; U.S. Pat. No. 5,672,510 involving retroviral vectors; and U.S. Pat. No. 5,635,399 involving retroviral vectors expressing cytokines.

Nonviral delivery methods include, but are not limited to, liposome-mediated transfer, naked DNA delivery (direct injection), receptor-mediated transfer (ligand-DNA complex), electroporation, calcium phosphate precipitation, and microparticle bombardment (e.g., gene gun). Gene therapy materials and methods may also include the use of inducible promoters, tissue-specific enhancer-promoters, DNA sequences designed for site-specific integration, DNA sequences capable of providing a selective advantage over the parent cell, labels to identify transformed cells, negative selection systems and expression control systems (safety measures), cell-specific binding agents (for cell targeting), cell-specific internalization factors, and transcription factors to enhance expression by a vector as well as methods of vector manufacture. Such additional methods and materials for the practice of gene therapy techniques are described in U.S. Pat. No. 4,970,154 involving electroporation techniques; WO96/40958 involving nuclear ligands; U.S. Pat. No. 5,679,559 describing a lipoprotein-containing system for gene delivery; U.S. Pat. No. 5,676,954 involving liposome carriers; U.S. Pat. No. 5,593,875 concerning methods for calcium phosphate transfection; and U.S. Pat. No. 4,945,050 wherein biologically active particles are propelled at cells at a speed whereby the particles penetrate the surface of the cells and become incorporated into the interior of the cells.

It is also contemplated that gene therapy or cell therapy according to the present invention can further include the delivery of one or more additional polypeptide(s) in the same or a different cell(s). Such cells may be separately introduced into the patient, or the cells may be contained in a single implantable device, such as the encapsulating membrane described above, or the cells may be separately modified by means of viral vectors.

A means to increase endogenous polypeptide expression in a cell via gene therapy is to insert one or more enhancer element into the polypeptide promoter, where the enhancer element(s) can serve to increase transcriptional activity of the gene. The enhancer element(s) used will be selected based on the tissue in which one desires to activate the gene(s); enhancer elements known to confer promoter activation in that tissue will be selected. Here, the functional portion of the transcriptional element to be added may be inserted into a fragment of DNA containing the polypeptide promoter (and optionally, inserted into a vector and/or 5′ and/or 3′ flanking sequence(s), etc.) using standard cloning techniques. This construct, known as a “homologous recombination construct”, can then be introduced into the desired cells either ex vivo or in vivo.

Gene therapy also can be used to decrease polypeptide expression by modifying the nucleotide sequence of the endogenous promoter(s). Such modification is typically accomplished via homologous recombination methods. For example, a DNA molecule containing all or a portion of the promoter of the gene selected for inactivation can be engineered to remove and/or replace pieces of the promoter that regulate transcription. For example the TATA box and/or the binding site of a transcriptional activator of the promoter may be deleted using standard molecular biology techniques; such deletion can inhibit promoter activity thereby repressing the transcription of the corresponding gene. The deletion of the TATA box or the transcription activator binding site in the promoter may be accomplished by generating a DNA construct comprising all or the relevant portion of the polypeptide promoter(s) (from the same or a related species as the polypeptide gene to be regulated) in which one or more of the TATA box and/or transcriptional activator binding site nucleotides are mutated via substitution, deletion and/or insertion of one or more nucleotides. As a result, the TATA box and/or activator binding site has decreased activity or is rendered completely inactive. The construct will typically contain at least about 500 bases of DNA that correspond to the native (endogenous) 5′ and 3′ DNA sequences adjacent to the promoter segment that has been modified. The construct may be introduced into the appropriate cells (either ex vivo or in vivo) either directly or via a viral vector as described herein. Typically, the integration of the construct into the genomic DNA of the cells will be via homologous recombination, where the 5′ and 3′ DNA sequences in the promoter construct can serve to help integrate the modified promoter region via hybridization to the endogenous chromosomal DNA.

Methods of Treatment

The identification and characterization of the polypeptide of SEQ ID No:2 as a modulator (i.e. repression or augmentation) of SRA regulated transactivation of nuclear receptors and the recognition of it's role in energy and metabolism means that there are a broad range of diseases and disorders that may be treated using therapies based on the polypeptides described herein.

Thus, the present invention provides a method for treating a disorder associated with an undesirable level of activation of a nuclear receptor, the method comprising the step of administering an effective amount of an isolated polypeptide comprising:
(i) SEQ ID No: 2;
(ii) amino acids 27 to 109 of SEQ ID No:2;
(iii) amino acids 22 to 109 of SEQ ID No:2;
(iv) amino acids 21 to 91 of SEQ ID No:2;
(v) amino acids 21-26 and/or 60-67 of SEQ ID No:2; or
(vi) a functional variant of any one of (i) to (v).

The nuclear receptor may be any receptor that is capable of being transactivated by SRA. The receptor may be selected from a nuclear receptor subfamily such as Type I NR—classically defined as ligand dependent and will homodimerize, Type II NR—classically defined as ligand independent and potential for both homo- and heterodimerization and orphan receptors-ligands that have only recently been characterized at the filing date of this application. Preferably, the receptor is selected from the group comprising: PPAR (e.g. alpha, beta/gamma and delta) RXR's, RAR's, ERRs (e.g. alpha, beta and gamma), estrogen receptor a (ERα), estrogen receptor β (ERβ), progesterone receptor (PR), androgen receptor (AR), glucocorticoid receptor (GR), mineralocorticoid receptor (MR), retinoic acid receptor α (RARα), retinoic acid receptor β (RARβ), retinoic acid receptor γ (RARγ), thyroid hormone receptor α (TRα), thyroid hormone receptor β (TRβ), vitamin D receptor (VDR), ecdysteroid receptor (EcR), retinoid X receptor α (RXRα), retinoid X receptor β (RXRβ), liver X receptor α (LXRα), liver X receptor β (LXRβ, farnesoid X receptor (FXR), pregnane X receptor (PXR), steroid and xenobiotic receptor (SXR) and constitutive andronstane receptor (CAR).

Glucocorticoid related disorders include Cushings disease or hyperadrenocorticism. Excessive levels of glucocorticoids can have widespread effects on metabolism and organ function. A diverse set of clinical manifestations accompany these disorders, including hypertension, ischaemic heart disease, dyslipidaemia (cholesterol and triglyceride abnormalities), apparent obesity, muscle wasting, thin skin, and metabolic aberrations such as diabetes.

Insufficient production of cortisol, often accompanied by an aldosterone deficiency, is called Addison's disease or hypoadrenocorucism. Most commonly, this disease is a result of infectious disease (e.g. tuberculosis in humans) or autoimmune destruction of the adrenal cortex. As with Cushing's disease, numerous diverse clinical signs accompany Addison's disease, including cardiovascular disease, lethargy, diarrhea, and weakness. Aldosterone deficiency can be acutely life threatening due to disorders of electrolyte balance and cardiac function.

Androgen receptor related disorders include: cancer such as prostate cancer, hirsuitism (or excessive hair growth), problems with libido and erectile dysfunction and osteoporosis.

Thyroid receptor related disorders include: overactivity of the thyroid (including Graves' disease, toxic multinodular goitre and toxic nodules) and underactivity of the thyroid (including Hashimoto's disease).

Vitamin D receptor related disorders include: Vitamin D deficiency or rickets, osteoporosis, and a role in differentiation and colonic carcinoma.

Therapeutic methods in accordance with the present invention will vary depending on whether the intention is to modulate by increasing or decreasing the amount and/or physiological effects of the polypeptides described herein, such as SEQ ID No:2. Hereunder is a range of methods that may be used to increase or decrease the effects of the polypeptide as deemed appropriate by a clinician with a view to achieving a therapeutic outcome.

Thus, the present invention also provides a method of treating a subject suffering from a disorder associated with undesirable physiological levels of a polypeptide of SEQ ID No:2 comprising the step of manipulating the physiological levels of the polypeptide. This manipulation may be intended to affect the activity of downstream signalling pathways and/or mitochondrial biogenesis and activity.

The physiological levels of the polypeptide can be increased or decreased as required to treat particular disorders. These increases or decreases can be achieved using the polypeptides, polynucleotides and/or selective binding agents described herein. These agents are capable of increasing or decreasing the endogenous production of the polypeptide or can be administered directly to increase or decrease the physiological levels of the polypeptide using the methods described herein. For example, selective binding agents, such as antibodies could be administered to decrease the physiological levels of the polypeptide of SEQ ID No:2 by binding the polypeptide and preventing it from binding SRA.

The polypeptide of SEQ ID No:2 regulates the action of hormones, including estrogen and glucocorticoids, and is expressed widely in human cancer cell lines. In addition, it is highly expressed in normal human skeletal muscle, liver and heart. Thus, disorders that may benefit from appropriate manipulation (increase or decrease) in the physiological levels of the polypeptide include: cancer such as hormone-dependent cancers e.g. prostate, breast, ovary, endometrium and other cancers including kidney cancer, or lung, bone, liver, colon or cervical cancer; disorders in which glucocorticoids are aberrant e.g. pituitary and adrenal disease that results in excess glucocorticoid production; disorders in which energy homeostasis is altered, including obesity, insulin resistance and diabetes mellitus; defects of fatty acid oxidation such as lipid storage myopathies; defects of the mitochondrial respiratory chain such as mitochondrial myopathies; the expression of the polypeptide in heart tissue also suggests a role in cardiac hormone action, in particular thyroid hormone signalling in the heart; additionally the detection of the thyroid hormone and estrogen receptor β nuclear receptors in the mitochondria suggests that the polypeptide may play a role in modulating thyroid and other hormone action in that organelle.

It may be preferable to administer the polypeptides, polynucleotides or binding agents of the present invention in combination with other therapeutic agents that are useful for treating a given disease or disorder. Such combinations could use conjugates comprising the polypeptide or the therapy could be concomitant or involve the sequential administration of the agents.

Applicant has found that the polypeptide of SEQ ID No: 2 can also enhance the repression of SRA binding mediated by SHARP (1). Thus, the present invention also comprises a method for enhancing the repression of SRA mediated by SHARP comprising the step of administering an effective amount of SHARP or a functional equivalent thereof and an isolated polypeptide comprising:
(i) SEQ ID No: 2;
(ii) amino acids 27 to 109 of SEQ ID No:2;
(iii) amino acids 22 to 109 of SEQ ID No:2;
(iv) amino acids 21 to 91 of SEQ ID No:2;
(v) amino acids 21-26 and/or 60-67 of SEQ ID No:2; or
(vi) a functional variant of any one of (i) to (v).

The above method may be particularly useful for treating disorders associated with an undesirable level of activation of a nuclear receptor, some of which are described more fully above.

With particular reference to treatment of cancer, such as breast cancer and prostate cancer, the polypeptides and/or polynucleotides herein may be used in combination with one or more agents that are adapted to interfere with the natural action of estrogen. These agents include antagonists (such as ICI 182,780) and SERMs (such as Tamoxifen and raloxifene) as well as agents that affect or inhibit the production of estrogen e.g. steroid ablation therapy. Other combination partners include agents that block other receptors on cancer cells that may be responsible for cell proliferation e.g. agents that activate erbB-2 or EGF-receptor. Estrogen antagonists are known to those skilled in the art and may be selected from the group consisting of: and ICI. Other inhibitors of estrogen production include aromatase inhibitors (e.g. Letrazole). These may also represent good combination partners for therapy according to the present invention.

Other potential therapies that could be combined with SLIRP based therapies include inhibitors of the tyrosine kinase signalling pathway (EGF-receptor monoclonal antibodies) or small molecule inhibitors such as Iressa. Inhibitors of the erbB-2 pathway may also be used (egg. Herceptin)

The effect of the administered therapeutic composition can be monitored by standard diagnostic procedures. For example, in the treatment of cancer, the patient outcomes from the administration of a therapeutic composition can be monitored by assessing any one or more of the recognized markers for disease progression.

The polypeptides may be administered as a therapeutic or a prophylactic depending on the particular circumstances and as deemed appropriate by a medical practitioner.

Diagnostics/Prognostics

The polypeptide, polynucleotides and binding agents herein may also be used for diagnostic and prognostic purposes in relation to any diseases or disorder associated with undesirable physiological levels of a polypeptide according to SEQ ID No:2.

Thus, the present invention also provides a method for performing a diagnosis on a patient comprising:
(a) determining the concentration of a polypeptide described herein in a biological sample, taken from the patient;
(b) comparing the level determined in step (a) to the concentration range of the polypeptide known to be present in normal subjects; and
(c) diagnosing whether the patient has the disorder based on the comparison in step (b).

The diagnostic method of the present invention can be used for any disorder associated with undesirable levels of the polypeptide of SEQ ID No:2. Preferably, step (a) above is performed using a binding agent described herein.

The diagnostic method may be applied to patients known to be suffering from a disorder associated with undesirable levels of the polypeptide according to SEQ ID No:2 with a view to assessing their response to treatment for the disorder.

Furthermore, the present invention may be applied to assess the prognosis of a patient. Thus, the present invention also provides a method for prognostic evaluation of a patient comprising:
(a) determining the concentration of a polypeptide described herein in a biological sample, taken from the patient;
(b) comparing the level determined in step (a) to the concentration range of the polypeptide known to be present in normal subjects; and
(c) evaluating the prognosis of said patient based on the comparison in step (b).

The prognostic method of the present invention can be used for any disorder associated with undesirable levels of the polypeptide of SEQ ID No:2 such as those mentioned herein.

The present invention may also be applied to assess the suitability of patients for particular therapeutic interventions. Thus, the present invention also provides a method for determining a therapeutic intervention in a patient with a disorder comprising:
(i) determining the concentration of a polypeptide according to SEQ ID No:2 in a biological sample, taken from the patient;
(ii) comparing the level determined in step (i) to the concentration range of the polypeptide known to be present in normal subjects; and
(iii) evaluating the therapeutic intervention for the disorder based on the comparison in step (ii).

The polynucleotides herein may also be used as cancer biomarkers. In this regard, using the polynucleotides to analyze allelic imbalances (or Loss of Heterozygosity: LOH), it is possible to compare DNA samples from paired normal and cancer tissues, and to examine whether specific genetic changes associated with the polynucleotides of the present invention in primary cancers can serve as biomarkers of metastatic potential. In this regard, the frequent finding of LOH in a malignancy/tumor is often an indication of the presence of a tumour suppressor gene whose loss (via either mutation or deletion) plays a significant role in the progression to cancer.

In general terms LOH analysis may be carried out by identifying a number of patients with primary cancer (such as breast), and then genotyping these with a number of genetic markers spanning Chr 14q24-14q25. The intensity of ratios of the two genetic alleles in normal/tumor DNA pairs can then be examined in genetically informative individuals. LOH can be scored based on the tumor allele intensity ratios.

Histological slides from routine, archival, clinical specimens can be screened microscopically for adequate amounts of control tissue and invasive metastatic breast cancers. Microdissection of specimens can then be performed on serial sections from formalin-fixed, paraffin-embedded tissue blocks. Following this, DNA can be liberated from the micro-dissected specimens.

Samples can then undergo PCR amplification as per normal laboratory practice. PCR products are then separated electrophoretically on 6.5% denaturing (8M urea) polyacrylamide gels, and subject to autoradiography. LOH is observed in tumours when one allele of DNA is dramatically reduced in intensity or lost entirely relative to remaining alleles in normal DNA.

Microsatellite marker information (against which primers can be developed and comparative analysis can occur) can be obtained from the Genethon Genetic Linkage Map (http://www.genethon.fr) and the Johns Hopkins University BioInformatics Genome Database (http://www.bis.med.jhmi.edu/). Their relative order can be determined via The Center of Medical Genetics Database (http://www2.marshfieldclinic.org/RESEARCH/GENETICS/).

Transgenics

The present invention also includes non-human animals such as mice, rats, or other rodents, rabbits, goats, or sheep, or other farm animals, in which the gene encoding the polypeptide of SEQ ID No:2 or a variant thereof has been disrupted (“knocked out) such that the level of expression of this gene or genes is(are) significantly decreased or completely abolished. Such animals may be prepared using techniques and methods such as those described in U.S. Pat. No. 5,557,032.

The present invention further includes non-human animals such as mice, rats, or other rodents, rabbits, goats, sheep, or other farm animals, in which either the native form of the gene encoding the polypeptide of SEQ ID No:2 or variant thereof for that animal or a heterologous gene is over-expressed by the animal, thereby creating a “transgenic” animal. Such transgenic animals may be prepared using well known methods such as those described in U.S. Pat. No. 5,489,743 and PCT application No. WO94/28122.

The present invention further includes non-human animals in which the promoter for one or more of the polypeptides of the present invention is either activated or inactivated (e.g., by using homologous recombination methods) to alter the level of expression of one or more of the native polypeptides.

These non-human animals may be used for drug candidate screening. In such screening, the impact of a drug candidate on the animal may be measured. For example, drug candidates may decrease or increase the expression of the gene encoding the polypeptide. In certain embodiments, the amount of the polypeptide, which is produced may be measured after the exposure of the animal to the drug candidate. Additionally, in certain embodiments, one may detect the actual impact of the drug candidate on the animal. For example, the overexpression of a particular gene may result in, or be associated with, a disease or pathological condition. In such cases, one may test a drug candidate's ability to decrease expression of the gene or its ability to prevent or inhibit a pathological condition. In other examples, the production of a particular metabolic product such as a fragment of a polypeptide, may result in, or be associated with, a disease or pathological condition. In such cases, one may test a drug candidate's ability to decrease the production of such a metabolic product or its ability to prevent or inhibit a pathological condition.

Set out hereunder is a general approach to the production of transgenic non-human animals.

A. Preparation of Constructs for Transformation

1. Selection of Transgene

The transgene of interest encoding a polypeptide or variant thereof of the present invention is selected. The simultaneous use of more than one transgene for insertion into a single embryo is within the scope of this invention. The structural gene may be obtained from any source, if obtained from vertebrate mammals, the structural gene may be from a homologous source (i.e., a gene from one mouse implanted into another mouse), or from a non-homologous source (i.e., a structural gene from rabbit implanted into a mouse). The transgene may have additional effects on the phenotype of the transgenic mammal, and these effects may be related or unrelated to the physiological action of the polypeptides herein. Preferred transgenes for use in the present invention include myc, myb, E2F (Nevins, J. R., Science 258:424429 [1992]), abl, ras, pim.1, src, E1A (Nevins, supra), HPVE7 (human papilloma virus E7, Nevins, supra), and SV40 early region, SV40 large T antigen, SV40 large T antigen tsA58 mutant, and mutants and fragments thereof. More preferred genes include myc, E2F, SV40 early region, and the SV40 early region tsA58 mutant. The most preferred gene is SV40 early region tsA58 mutant.

2. Selection of Promoter

Promoters useful in practicing this invention are those that are highly regulated with respect to activity, both temporally and spatially. Thus, the promoters of choice are those that are active only in particular tissues or cell types. The source of the promoter may be from any unicellular prokaryotic or eukaryotic organism, any vertebrate or invertebrate, or any plant. Where the promoter is obtained from a mammal, the mammal may be homologous (the same species as the mammal to be transfected) or non-homologous (a different species).

3. Other Vector Components

In addition to a promoter and one or more structural genes, the vectors of this invention preferably contain other elements useful for optimal functioning of the vector in the mammal into which the vector is inserted. These elements are well known to those of ordinary skill in the art, and are described, for example in Sambrook et al., Molecular Cloning: A Laboratory Manual Cold Spring Harbor Laboratory Press, 1989.

4. Construction of Vectors

Vectors used for transforming mammalian embryos are constructed using methods well known in the art, including, without limitation, the standard techniques of restriction endonuclease digestion, ligation, plasmid and DNA and RNA purification, DNA sequencing, and the like as described, for example in Sambrook, Fritsch, and Maniatis, eds., Molecular Cloning: A Laboratory Manual., (Cold Spring Harbor Laboratory Press, Cold Spring Harbor, N.Y. [1989]).

B. Production of Transgenic Mammals

The specific lines of any mammalian species used to practice this invention are selected for general good health, good embryo yields, good pronuclear visibility in the embryos, and good reproductive fitness. For example, when transgenic mice are to be produced, lines such as C57/BL6×DBA2 F1 cross, or FVB lines are used (obtained commercially from Charles River Labs).

The age of the mammals that are used to obtain embryos and to serve as surrogate hosts is a function of the species used, but is readily determined by one of ordinary skill in the art. For example, when mice are used, pre-puberal females are preferred, as they yield more embryos and respond better to hormone injections.

Similarly, the male mammal to be used as a stud will normally be selected by age of sexual maturity, among other criteria.

Administration of hormones or other chemical compounds may be necessary to prepare the female for egg production, mating, and/or reimplantation of embryos. The type of hormones/cofactors and the quantity used, as well as the timing of administration of the hormones will vary for each species of mammal. Such considerations will be readily apparent to one of ordinary skill in the art.

Typically, a primed female (i.e., one that is producing eggs that can be fertilized) is mated with a stud male, and the resulting fertilized embryos are then removed for introduction of the transgene(s). Alternatively, eggs and sperm may be obtained from suitable females and males and used for in vitro fertilization to produce an embryo suitable for introduction of the transgene.

Normally, fertilized embryos are incubated in suitable media until the pronuclei appear. At about this time, exogenous nucleic acid comprising the transgene of interest is introduced into the female or male pronucleus. In some species such as mice, the male pronucleus is preferred.

Introduction of nucleic acid may be accomplished by any means known in the art such as, for example, microinjection. Following introduction of the nucleic acid into the embryo, the embryo may be incubated in vitro for varying amounts of time, or reimplanted into the surrogate host, or both. In vitro incubation to maturity is within the scope of this invention. One common method is to incubate the embryos in vitro for 1-7 days and then reimplant them into the surrogate host.

Reimplantation is accomplished using standard methods. Usually, the surrogate host is anesthetized, and the embryos are inserted into the oviduct. The number of embryos implanted into a particular host will vary, but will usually be comparable to the number of offspring the species naturally produces.

Transgenic offspring of the surrogate host may be screened for the presence of the transgene by any suitable method. Screening is often accomplished by Southern or Northern analysis, using a probe that is complementary to at least a portion of the transgene. Western blot analysis using an antibody against the protein encoded by the transgene may be employed as an alternative or additional method for screening. Typically, the tissues or cells believed to express the transgene at the highest levels are tested, although any tissues or cell types may be used for this analysis.

Alternative or additional methods for evaluating the presence of the transgene include, without limitation, suitable biochemical assays such as enzyme and/or immunological assays, histological stains for particular markers or enzyme activities, and the like. Blood cell count data is useful for evaluation of thrombocytopenia.

Progeny of the transgenic mammals may be obtained by mating the transgenic mammal with a suitable partner, or by in vitro fertilization of eggs and/or sperm obtained from the transgenic mammal. Where in vitro fertilization is used, the fertilized embryo may be implanted into a surrogate host or incubated in vitro, or both. Where mating is used to produce transgenic progeny, the transgenic mammal may be backcrossed to a parental line. Using either method, the progeny may be evaluated for the presence of the transgene using methods described above, or other appropriate methods.

The transformed mammals, their progeny, and cell lines of the present invention provide several important uses that will be readily apparent to one of ordinary skill in the art. The mammals and cell lines are particularly useful in screening compounds that have potential as prophylactic or therapeutic treatments for diseases associated with undesirable levels of the polypeptide of SEQ ID No:2 or a variant thereof such as cancer or energy or metabolic disorders.

In the case of transgenic mammals, screening of candidate compounds is conducted by administering the compound(s) to be tested to the mammal, over a range of doses, and evaluating the mammal's physiological response to the compound(s) over time. Administration may be oral, or by suitable injection, depending on the chemical nature of the compound being evaluated. In some cases, it may be appropriate to administer the compound in conjunction with co-factors that would enhance the efficacy of the compound.

In screening cell lines for compounds useful in treating thrombocytopenia and/or megakaryocytic leukemia, the compound is added to the cell culture medium at the appropriate time, and the cellular response to the compound is evaluated over time using the appropriate biochemical and/or histological assays. In some cases, it may be appropriate to apply the compound of interest to the culture medium in conjunction with co-factors that would enhance the efficacy of the compound.

General

Those skilled in the art will appreciate -that the invention described herein is susceptible to variations and modifications other than those specifically described. It is to be understood that the invention includes all such variations and modifications. The invention also includes all of the steps, features, compositions and compounds referred to or indicated in the specification, individually or collectively and any and all combinations or any two or more of the steps or features.

The present invention is not to be limited in scope by the specific embodiments described herein, which are intended for the purpose of exemplification only. Functionally equivalent products, compositions and methods are clearly within the scope of the invention as described herein.

The entire disclosures of all publications (including patents, patent applications, journal articles, laboratory manuals, books, or other documents) cited herein are hereby incorporated by reference. No admission is made that any of the references constitute prior art or are part of the common general knowledge of those working in the field to which this invention relates.

Throughout this specification, unless the context requires otherwise, the word “comprise”, or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

### Examples

General Materials/Methods

(i) Cell Culture

The cell lines (MCF-7, MDA-MB-468, SK-BR-3, T47D, LNCaP, 22Rv1, PC3, HepG2, Calu-6, HT1080, COS-7, HeLa) were obtained from the American Type Culture Collection. C2C12 cells were obtained from Dr George Muscat. HMEC cells were obtained from Dr. Roger Reddell. Cells were grown as per manufacturer's recommendations and as previously described (17), and used within 20 passages of the original stock for all experiments.

(ii) Yeast Three-Hybrid Screen of Human Breast Cancer Library

The pIIIA/MS2-2 plasmid and the Saccharomyces cerevisiae L40 coat used in the yeast-three hybrid screen were gifts of Dr. Marvin Wickens. Dr. Jennifer Byrne kindly donated the human breast cancer cDNA library (5). The screening protocol used was as described (3). Analysis of SRA and STR7 was performed using the Mfold program (15). From the initial screen 150 colonies underwent strict stringency tests as described (6). Plasmid DNA was isolated from the yeast as described (3).

(iii) SLIRP Plasmid Constructs, Sequences and Vectors

The yeast three-hybrid SLIRP cDNA clone containing the coding region and 3′ UTR, was amplified with specific primers SLIRP (sense) 5′ cgc gga tcc gcg gcc tca gca gca 3′, SLIRP (reverse) 5′ gcg cgg atc cta ggc tgc agt ctca 3′, and subcloned into BamHI cut pCMV-FLAG 7.1 for transfection assays and pGEX-6P2 for GST-fusion protein experiments. STR7 primers (sense) 5′ agg agg cag gta tgt gat gac atc agc cga cgc ctg gca ctg ctg cag gaa cag tgg gct gga gga aag ttg tca ata cct gta aag aa 3′ and (reverse) 5′ ttc ttt aca ggt aft gac aac ttt cct cca gcc cac tgt tcc tgc agc agt gcc agg cgt cgg ctg atg tca tca cat acc tgc ttc ct 3′ were annealed and cloned into EcoRV-digested pBluescript II KS+ to generate labeled riboprobes. To generate the SRA mutant SDM7, the underlined residues above were mutated to tcc, ctc and ctc as outlined previously (19). pBluescript vectors were linearized with EcoRV for transcription with T7 or T3 RNA polymerase (Invitrogen) in reactions containing [³²P]UTP (Amersham Biosciences) or unlabeled UTP, as described (16), to produce riboprobes with a specific activity of ˜2×10⁹ cpm/μg RNA. Constructs for expressing mutant forms of SLIRP (R24, 25A and L62A) were prepared by PCR-based mutagenesis. Each of the mutant coding domains was subcloned into BamH1-cut pCMV-FLAG 7.1 and pGEX-6P2 as above. Sequence alignment comparison of SLIRP and SHARP, and SLIRP inter-species comparisons were performed using algorithms previously described (22).

(iv) REMSA and UVXL

REMSA and UVXL were performed as previously described (16). In RNA competition REMSA, excess (100 fold) unlabeled sense RNA transcript (pBluescript II KS+ vector (Stratagene) alone−pblue, pBluescipt-STR7, or tRNA (16) was incubated with the extract/protein sample for 30 min at 22° C. prior to incubation with the ³²P labeled probe. Samples were resolved by a 5% non-denaturing PAGE and visualized by PhosphorImager and ImageQuant software (Molecular Dynamics). Large scale pGEX-6P2 (GST alone, pGEX-SLIRP, pGEX-SLIRP R24, 25A and L62A, pGEX-SHARP-RRM and pGEX-SHARP-RD (1)) expression was performed according to standard protocols (17). To generate cleaved proteins, beads were incubated with PreScission Protease (Sigma).

(v) Transfection and Luciferase Assays

HeLa cells were transfected using FuGENE6 (Roche) with equal molar ratios of control and plasmid DNA: 50 ng/well ER(α); 0.6 μg/well ERE-Luc, GRE-Luc, TRE-Luc, ARE-Luc, VDRE-Luc; 0.0625 μg/well pSCT or 0.08 μg/well pSCT-SRA/pSCT-SRA-SDM7a; 0.049 μg/well pCMV-FLAG-7.1 or 0.050.5 μg/well FLAG-SLIRP/FLAG-SLIRP-R24/FLAG-SLIRP-L62/FLAG-SLIRP-DM; 0.3 μg/well pCMX or 0.5 μg/well pCMX-SHARP; 100 ng/well pCGN or 100 ng/well pCGN-SKIP. After transfection, cells were cultured in complete medium for 24 h, prior to the addition of E2 (10 nM), Dex (10 nM), T3 (1 nM), DHT (10 nM), VitD (100 nM), 4-hydroxytamoxifen (Tam, 1 nM), ICI182780 (ICI, 1 nM) or equal volume of vehicle (absolute ethanol or water). After 8 h, lysates were assayed for luciferase activity relative to protein levels using Luciferase (Promega) and Protein Assay (BioRad) on a FluorStar Optima (BMG).

(vi) SLIRP RNA Interference (siRNA)

siRNA complexes directed against SRA, SLIRP or control non-targeting (Dharmacon RNA Technologies USA, final concentration 20-200 nM) were transfected into cells with Lipofectamine 2000 (Invitrogen). In assessing effects of SLIRP knock down on GRE-luc activity, the reporter plus pSCT or pSCT-SRA and the siRNA conjugates were added simultaneously. These cultures were grown without manipulation for a further 48 h, induced with Dex (10 nM) and lysates harvested and processed as described above.

(vii) Northern Analysis

Human tissue Northern blots were obtained from BD Biosciences (7759-1, 7760-1). Total RNA was isolated using TRIzol LS reagent (GibcoBRL), and poly A RNA prepared using biotinylated oligo (dT) primers and streptavidin-coupled magnetic particles (Poly ATtract™, Promega). Samples (2 μg) were electrophoresed and transferred to Hybond-N membrane (Amersham). Human SLIRP, SKIP or β-actin cDNAs were labeled (random primer. DNA labeling kit (GibcoBRL) incorporating [³²P]dCTP (Amersham)), hybridized with labeled probes (Rapid Hyb solution (Amersham) for 2 to 5 h), washed (to 0.1×SSC/0.1% SDS at 65° C.), and transcripts visualized by Phospholmager.

(viii) Western Immunoblotting

Cell lysates were resolved by SDS-PAGE and transferred onto PVDF Membrane (Roche). To detect SLIRP, rabbit polyclonal antisera raised against GST-SLIRP protein and was used at 1:250 as previously described (Giles et al., 2003). SRC-1 antibody (SCM-341), SKIP antibody (kind gift of Dennis Dowhan), ERα antibody (SC7207) and β-actin antibody (Abeam antibody 6276) were used with HRP conjugated anti-rabbit and anti-mouse secondary abs and ECL Plus (Amersham) to visualize target proteins.

(ix) Immunohistochemistry

Full-face sections of primary human breast cancer tissue were immunostained for SLIRP or HSP-60 using standard protocols. SLIRP polyclonal antibody or HSP-60 antibody (SC13115) was used at 1:2500, and biotinylated goat anti-rabbit and anti-mouse antibody (Chemicon IHC Select) followed by streptavidin-horseradish peroxidase used as secondary and tertiary reagents. Sections were vizualized with diaminobenzidine (DAKO) followed by a light counterstain with haematoxylin.

(x) Immunoprecipitation RT-PCR Assay

Method was as described (17). Using MCF-7, MDA-MB468 or HeLa cells with either SLIRP antibody, SRC-1 antibody, β-actin antibody or no antibody, co-immunopurifying SRA was detected using primers: SRAEIV (sense): 5′-tga tga cat cag ccg acg cct-3′ and SRAEIV(reverse) 5′-gct gca gat ttc tct tca ttg-3′. SLIRP (sense) 5′-gcg ctg cgt aga agt atc aa-3′ and SLIRP (reverse) 5′-tcg att ccg aag tcc ttc tt-3′. Actin (sense) 5′-gcc aac aca gtg ctg tct gg-3′ and actin (reverse) 5′-tac tcc tgc ttg ctg atc ca-3′.

(xi) Chromatin Immunoprecipitation (ChIP) Assay

The approach was as described (12). Briefly, cells (MCF-7 or HeLa) were treated (100 nM E2 for 0-120 min or Dex 100 nM for 15 min), fixed with 1% formaldehyde, lysed, sonicated and a proportion of the soluble chromatin used as an “input control”. Soluble chromatin was incubated with 4 μg antibody (ERα (SC7207), SLIRP polyclonal antibody, HuD (SC5979), SRC-1 (SC6096) or GR (a 50:50 combination of SC1002 and SC1004)), washed and recovered DNA fragments amplified with PCR using either pS2 primers: (sense) 5′-ggc cat ctc tca cta tga atc act tct gc-3′ and (reverse) 5′-ggc agg ctc tgt ttg ctt aaa gag cg-3′ or metallothionein (MTA2) primers: (sense) 5′-act cgt ccc ggc tct ttc ta-3′ and (reverse) 5′-agg agc agt tgg gat cca t-3′.

(xii) Imaging Studies

HeLa cells were cultured overnight on glass cover slips and incubated with MitoTracker (Molecular Probes), SLIRP, HSP-60 or cytochrome c (SC7159) antibody for 1 h, washed and Alexa Fluor 488 goat-anti-rabbit (Molecular Probes) secondary antibody added. Cover slips were bathed in 100 ng/ml Hoechst 33258/PBS, washed and mounted in Vectashield (Vector Laboratories). For the SLIRP-FLAG imaging studies, HeLa cells were transfected with the FLAG-tagged vector, treated similarly to above, and then stained with either MitoTracker or Cytochrome C antibody. Mounted cells were visualized using a BioRad MRC1000 confocal microscope using Confocal Assistant and Adobe Photoshop software.

(xiii) Polyclonal Sera Preparation

To prepare polyclonal antisera against GST-SLIRP, the methods described in “Antibodies a Laboratory Manual” were employed (Editors, Ed Harlow and David Lane; Cold Spring Harbor Laboratory, 1988, USA ISBN 0-87969-314-2). In brief, protein lysates from bacterial cultures expressing the protein were resolved by polyacrylamide gel electrophoresis and the region containing the fusion protein cut from the gel. This material was macerated and mixed with Freund's complete adjuvant prior to injection into rabbits. Animals were injected again after 4 weeks and then again after a further 2 weeks using GST-SLIRP protein mixed with Freund's incomplete adjuvant. Whole blood was removed from immunized animals approximately 10 weeks after the first injection. Following clotting, samples were centrifuged and the polyclonal sera isolated, aliquoted and stored at −80° C.

### Example 1

### SRA STR7 is a Target for Proteins in Human Breast Cancer Cells

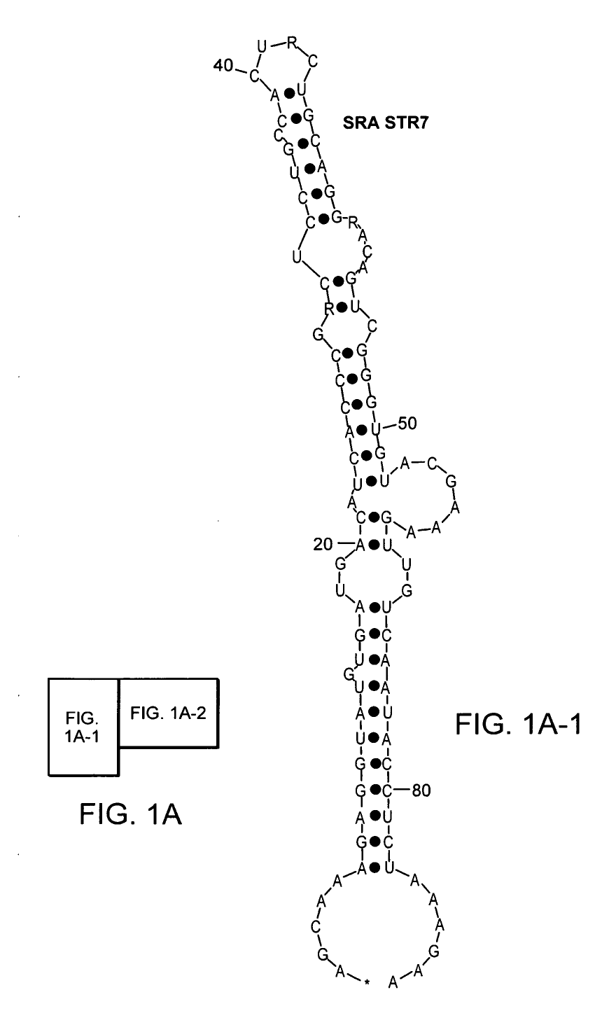

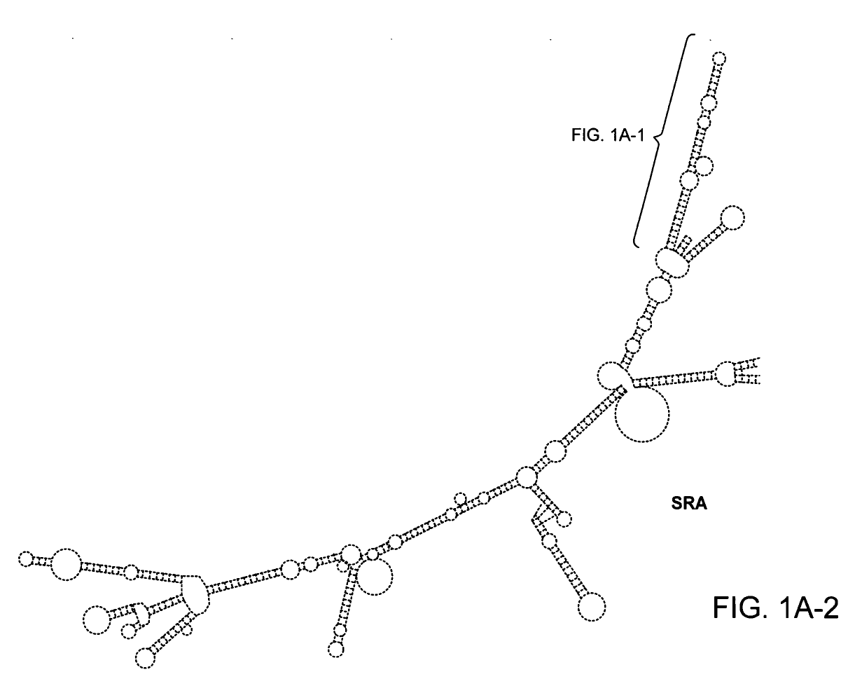

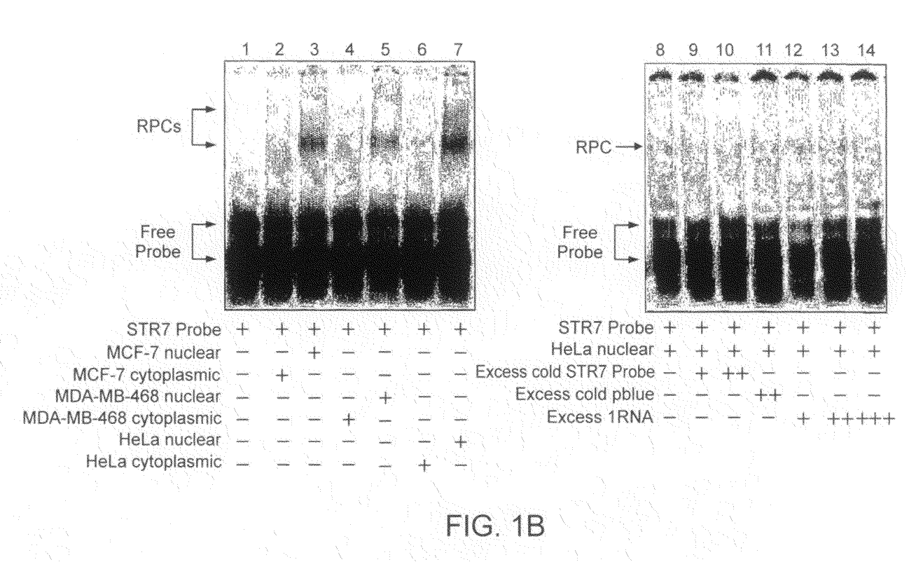

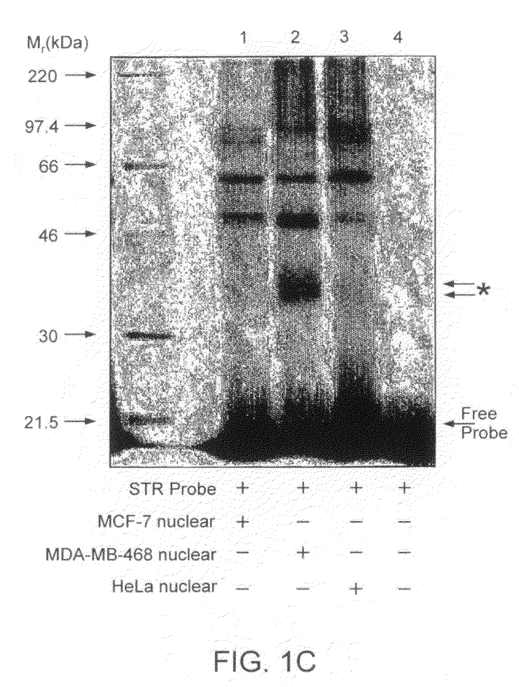

SRA is a complex RNA molecule with multiple stable stem-loop structures predicted by secondary (2⁰) structure analysis (FIG. 1A) (19). STR7, an 89-nucleotide (nt) sequence, is the largest and one of the most stable stem-loop SRA structures that functions in a cooperative manner with other stem-loops to augment transactivation of an E2-responsive reporter (19).

We first investigated whether SRA STR7 was a target for RNA-binding proteins in RNA electrophoretic mobility shift assay (REMSA) studies. While weak RNA-protein complexes (RPCs) were visible in the cytoplasmic extracts from several different cancer cell lines (MCF-7, MDA-MB468 and HeLa), two strong RPCs were evident in the nuclear extracts of each cell line (FIG. 1B). Addition of excess unlabeled STR7 effectively abrogated RPC formation (FIG. 1B). However, addition of 100-fold excess of either unlabeled vector transcript (pBluescript), or yeast tRNA competitor RNA did not diminish the formation of RPCs in HeLa cell nuclear extracts (FIG. 1B). Taken together, these data indicate a highly specific interaction between SRA STR7 and nuclear proteins from human cancer cells.

UV cross-link (UVXL) was performed to further characterize RPC formation with the STR7 riboprobe. Multiple STR7-protein complexes were identified from each of the nuclear cell extracts (FIG. 1C). Although many of the bands were common, there were some significant differences between cell types. For example, two RPCs of ˜39 and 40 kDa bound with a greater intensity in extracts from MDA-MB-468 cells compared to extracts from MCF-7 and HeLa cells (FIG. 1C, lane 2). These data show that an array of nuclear proteins bind SRA STR7 in vitro.

### Example 2

### Cloning of an SRA-Binding Protein (“SLIRP”)

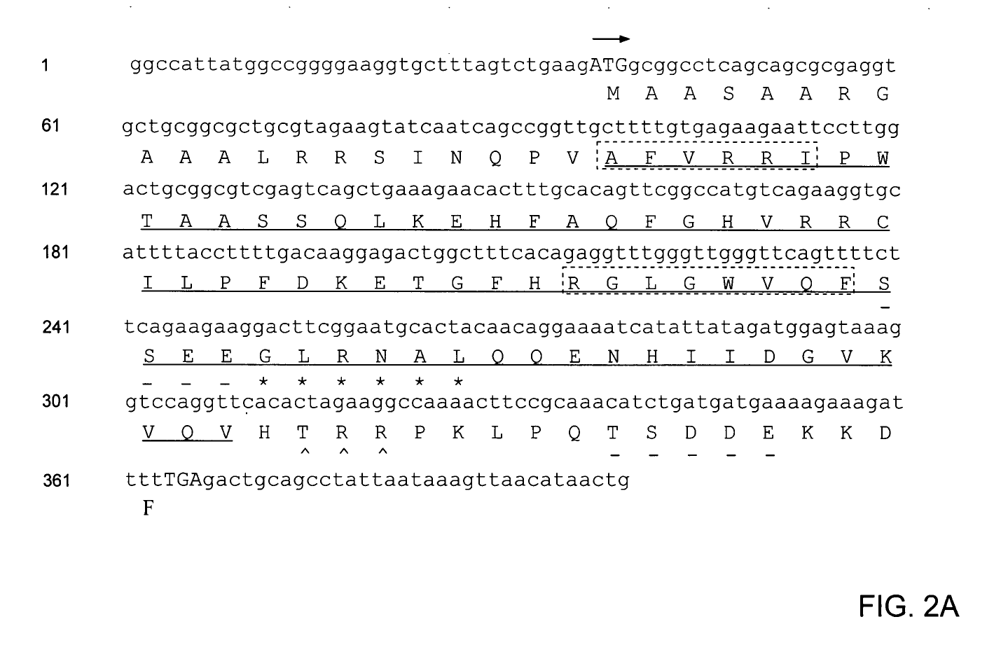

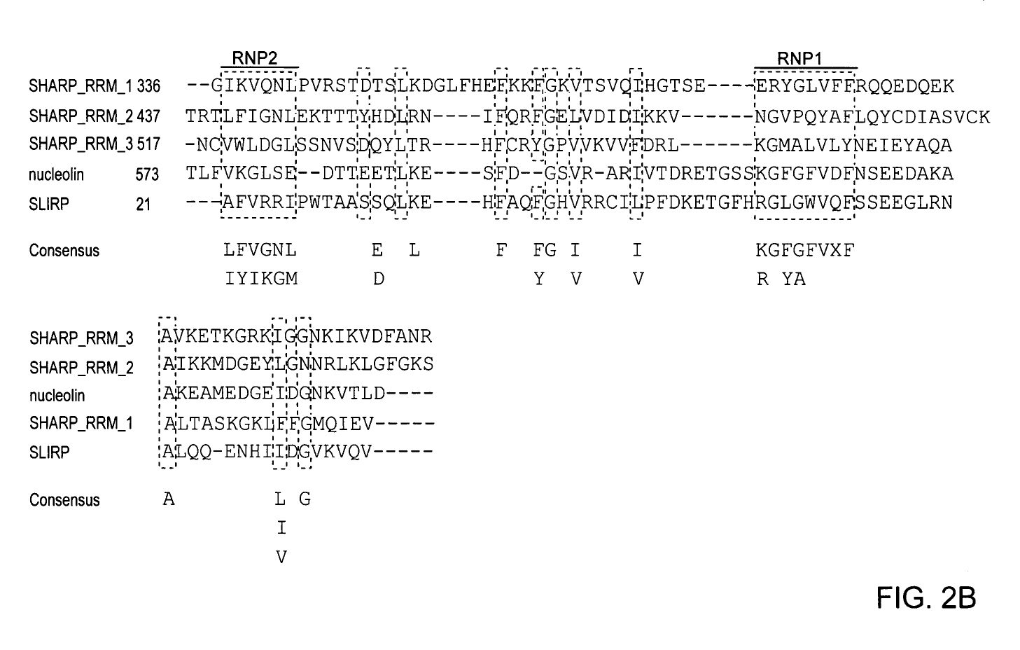

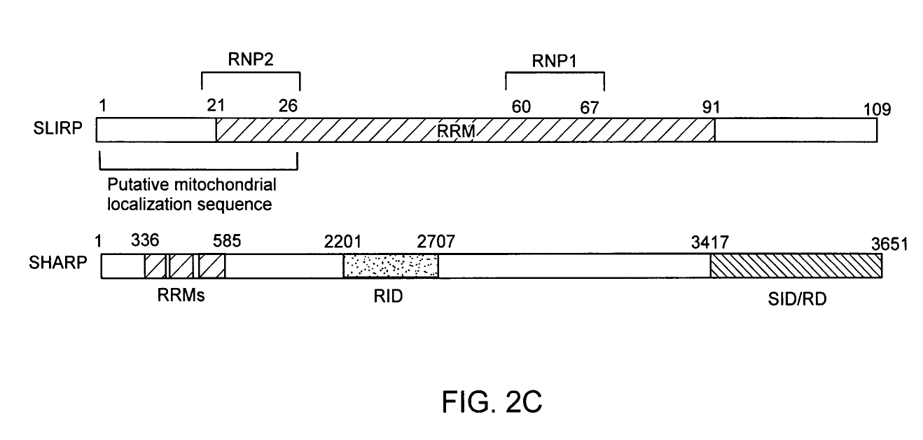

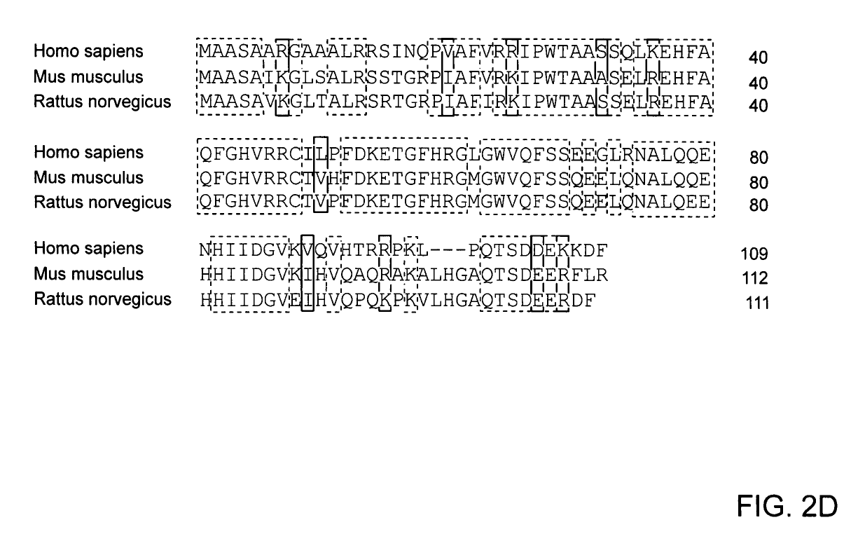

To isolate novel SRA-binding proteins, we used SRA STR7 as bait in a yeast three-hybrid screen (3) of a primary human breast cancer cDNA library (5). Mfold 2⁰ structure analysis indicated that the STR7 stem-loop structure was strictly preserved in the hybrid RNA pIIIA/MS2-2 bait construct. From the screen we isolated a cDNA clone that contained an open reading frame (minus the first methionine), a 3′ untranslated region (UTR) and polyadenylated (poly A) tail (FIG. 2A). The cDNA sequence (SEQ ID No:1) predicted a protein of 109 amino acids (aa), with a Mᵣ of 12.7 kDa (SEQ ID No:2).

The predicted protein sequence of SLIRP is composed almost entirely of an RNA recognition motif (RRM) (FIG. 2A) containing RNP1 and RNP2 submotifs (7). The RRM domain in SLIRP shares substantial aa homology with SHARP (FIG. 2B), a recently discovered SRA corepressor (1) and with nucleolin, a canonical RRM-containing protein (9).

In addition to its RRM, SLIRP contains a number of additional putative functional domains including a mitochondrial localization sequence (aa 1-26), a protein kinase C phosphorylation site (aa 9397), casein kinase II phosphorylation sites (aa 68-71, 101-104 and 102-105) and a N-myristoylation site (aa 72-77) (FIG. 2C). The aa sequence of SLIRP is highly conserved across species, including the rat and mouse genomes (FIG. 2D). Of interest, the mouse and rat SLIRP homologs are each surrounded by the same genes as human SLIRP.

Bioinformatic analysis of human SLIRP showed that it colocalizes to the same chromosomal position as SKIP, on Chr 14q24.3. There are no intervening genes in the 1750 nt separating SLIRP and SKIP. SKIP is a well described coregulator that acts as a corepressor in the VitD pathway, and has been implicated in oncogenesis (20, 21). SKIP is expressed in breast cancer tissue, and has been shown to regulate ER transactivation (20).

### Example 3

### SLIRP is Expressed Widely in Human Tissues and Cancer Cells

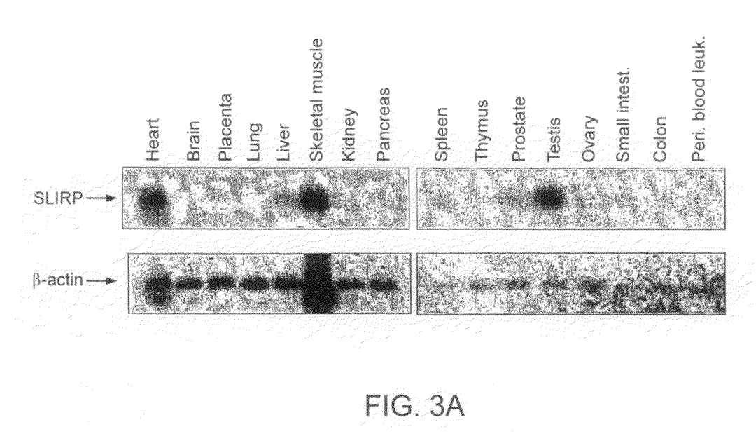

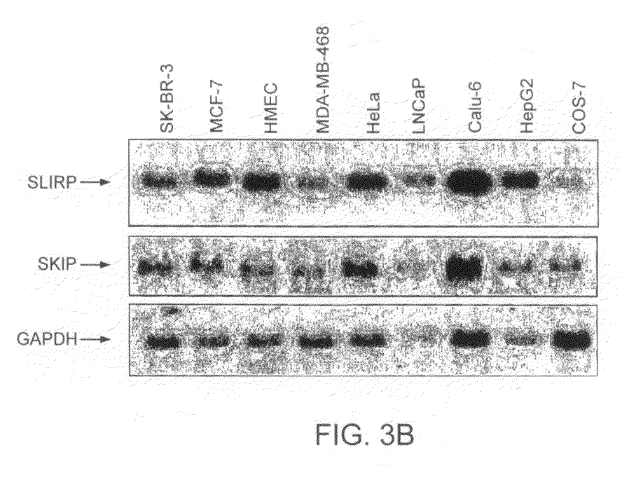

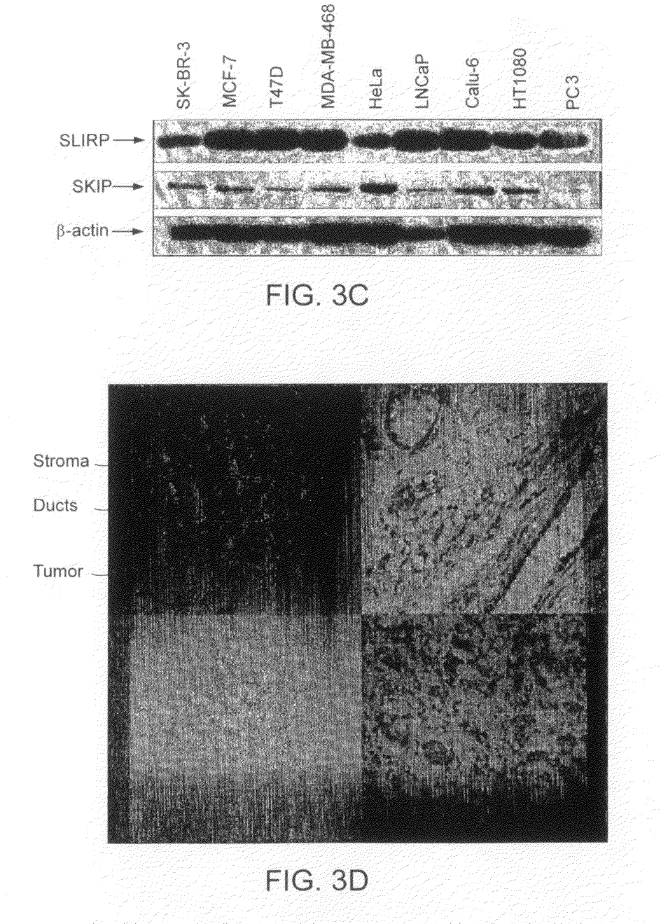

In normal human tissue, SLIRP mRNA is ubiquitously expressed, but in varying amounts. Transcripts were abundant in the heart, liver, skeletal muscle and testis (FIG. 3A). SLIRP was readily detected in a variety of cancer cell lines, including SK-BR-3, MCF-7, HMEC, MDA-MB-468, HeLa, LNCaP, Calu-6, HepG2 and COS-7 (FIG. 3B), and noticeably increased in HeLa (human cervical cancer), Calu-6 (human lung cancer) and HepG2 (human liver cancer) cells. Notably, we found that the relative expression levels of SLIRP across multiple tissues (FIG. 3A) and multiple cancer cell lines (FIG. 3B) were similar to those reported for SRA (19).

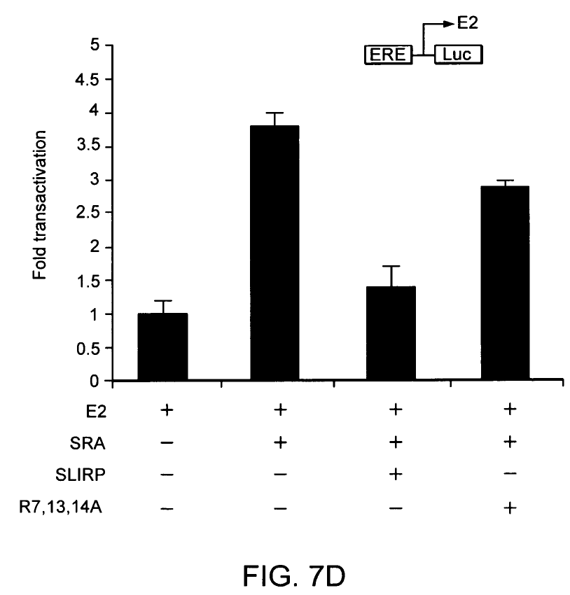

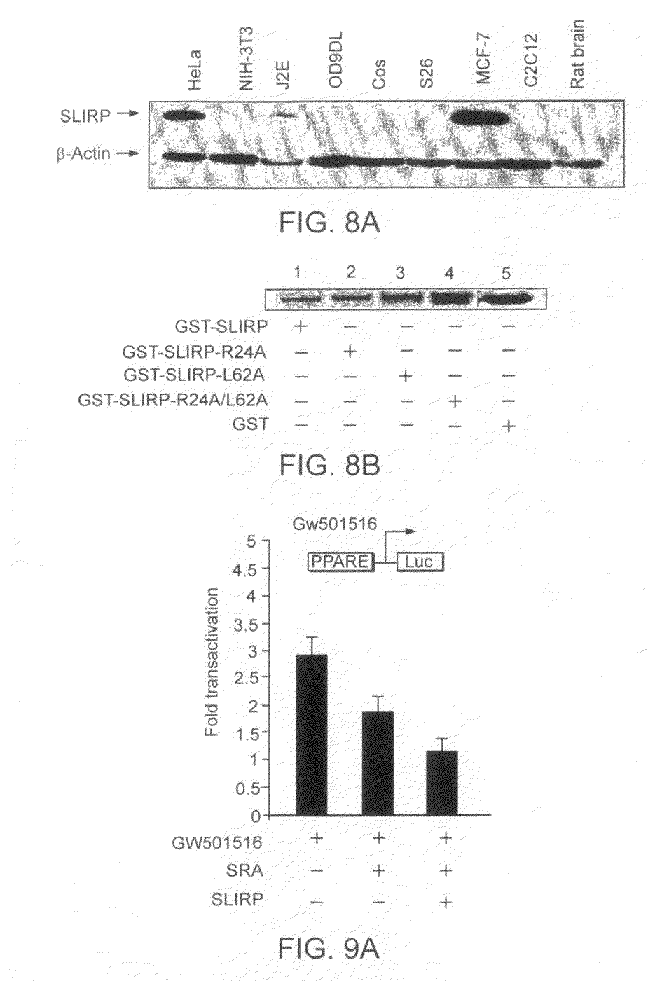

We generated a polyclonal SLIRP antibody and demonstrated SLIRP protein (Mᵣ ˜12.7 kDa) expression in multiple human cancer cell lines, including those derived from breast, prostate and lung carcinomas (FIG. 3C). The antibody was very specific for human SLIRP, with virtually no expression in a variety of other species (FIG. 8A). Expression of SLIRP protein varied across different breast cancer cell lines, and in some cells discordant levels of SLIRP mRNA and protein were observed (eg. HeLa cells).

Immunohistochemistry (IHC) with the SLIRP antibody on human primary breast cancer tissue showed SLIRP staining some normal ductal tissue, but little of the surrounding stroma (FIG. 3D, a & b). Intense SLIRP staining was noted in carcinoma tissue (FIG. 3D, d) when compared to the control (FIG. 3D, c). Staining was evident throughout the cell, but predominantly with punctate distribution throughout the cytoplasm (FIG. 3D, b & d).

### Example 4

### Characterization of SLIRP's Interaction with SRA

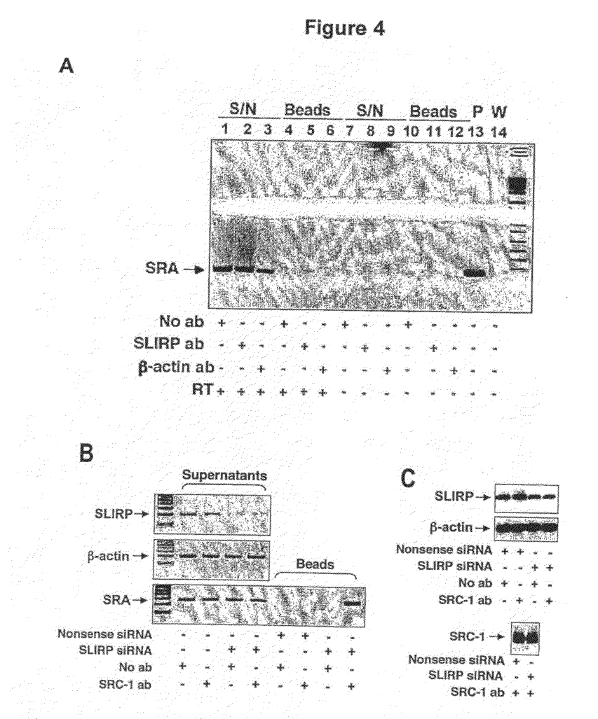

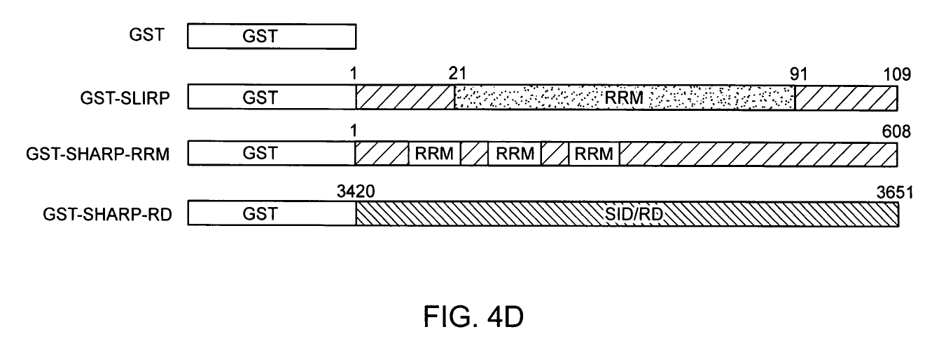

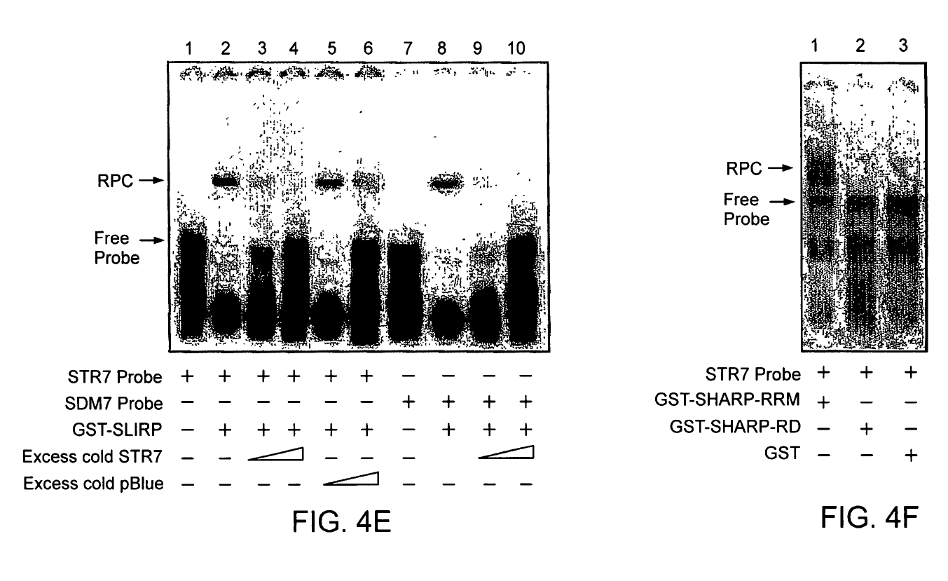

To confirm SLIRP's interaction with SRA in vivo, we performed immunoprecipitation-RT-PCR (IP-RT-PCR) assays with the SLIRP antibody. Using HeLa (FIG. 4A), MCF-7 and MDA-MB468 cells (data not shown), we found that SRA coimmunopurifies with SLIRP (FIG. 4A, lane 5), but not β-actin (FIG. 4A, lane 6). As SRA also co-purifies with SRC-1 (19), we next examined the effects on this interaction of reducing intracellular SLIRP levels with siRNA. When endogenous SLIRP expression was reduced, we found a corresponding increase in SRC1 associated with SRA (FIGS. 4B & C).

To assess the binding of SLIRP to STR7 in vitro, we performed REMSA with recombinant GST-SLIRP fusion and cleaved SLIRP proteins (FIG. 4D). Both GST-SLIRP (FIG. 4E, lane 2) and cleaved SLIRP (data not shown) bound STR7 avidly in the presence of heparin and RNase T1, while GST did not (FIG. 4F, lane 3). Addition of increasing amounts of unlabeled (cold) STR7 probe efficiently competed out the complex (FIG. 4E, lanes 3 & 4). In contrast, neither addition of excess unlabeled pBluescript transcript (FIG. 4E, lanes 5 & 6), nor inclusion of high amounts of tRNA affected SLIRP-STR7 complex formation (data not shown). Taken together, these results indicate that SLIRP binds STR7 in vitro with a high degree of specificity. We next tested binding of SLIRP to a mutant SRA STR7 probe (SDM7), which contains several point mutations within the stem-loop structure and is known to reduce transactivation activity of SRA (19). With an equivalent input of probe, binding of GST-SLIRP to the SRA SDM7 probe was reduced compared to the wild-type STR7 probe (FIG. 4E, lane 8). Binding to the mutant probe could also be overcome with excess unlabeled SRA STR7 (lanes 8 & 9).

Given the homology between SLIRP and SHARP within their RRM domain, we next examined if SHARP also bound SRA STR7. Using a GST-SHARP fusion protein (1) containing the three RRM domains (GST-SHARP-RRM), we found that it bound STR7 avidly under stringent conditions (FIG. 4F, lane 1). In contrast, GST-SHARP-RD fusion protein (containing the repression domain of SHARP) did not bind STR7 (FIG. 4F, lane 2). These data indicate that the RRM domain of SHARP may compete with SLIRP for binding to SRA STR7.

### Example 5

### SLIRP Represses SRA-Mediated Nuclear Receptor Coactivation

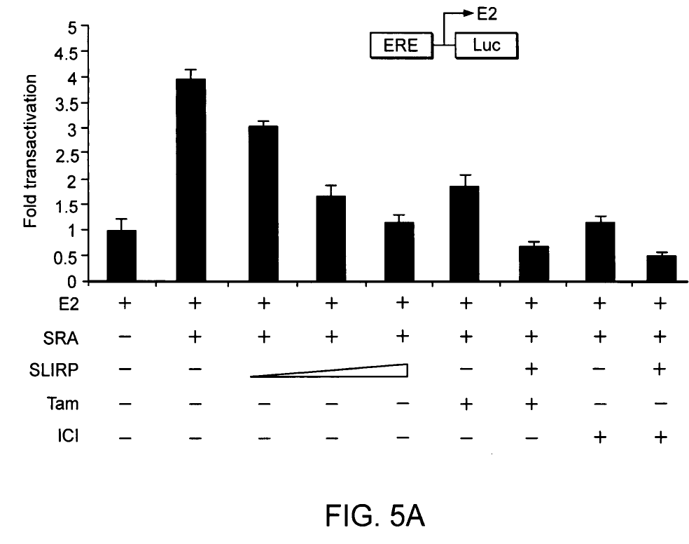

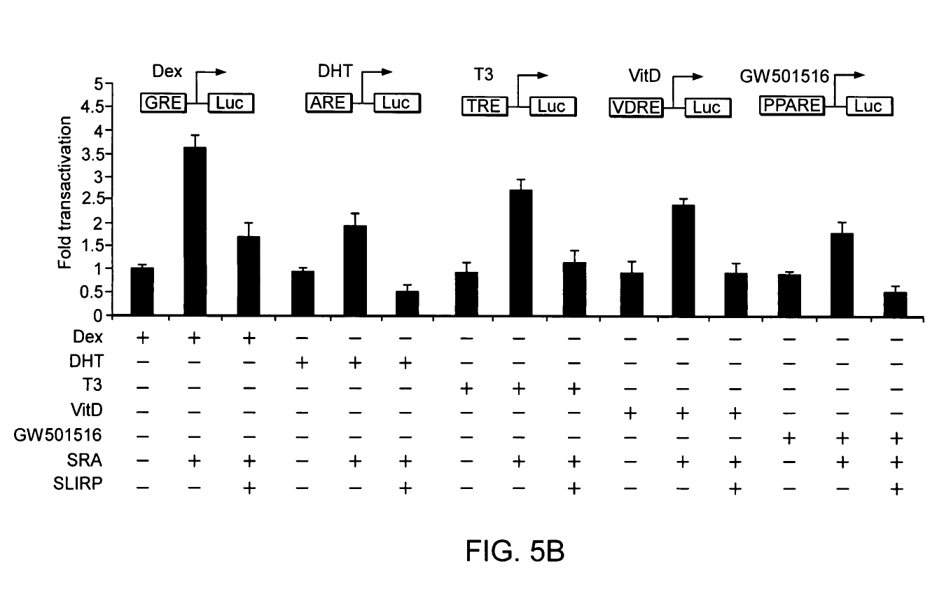

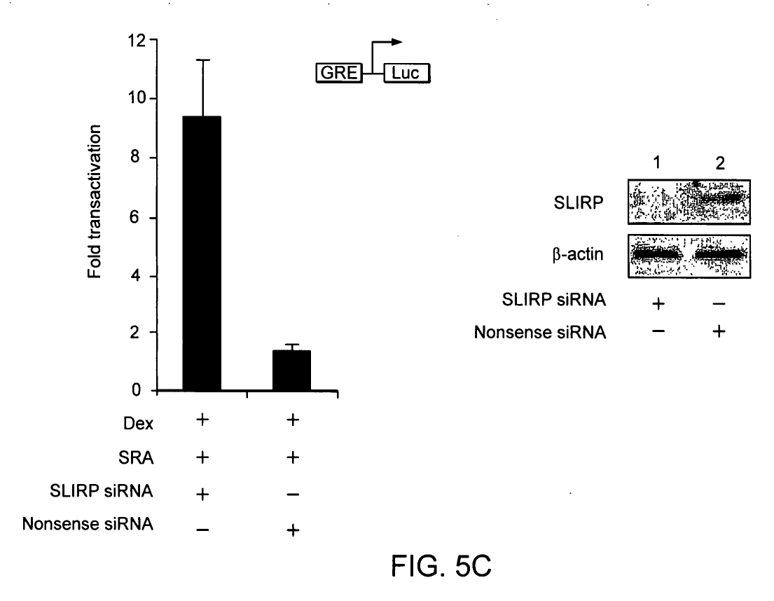

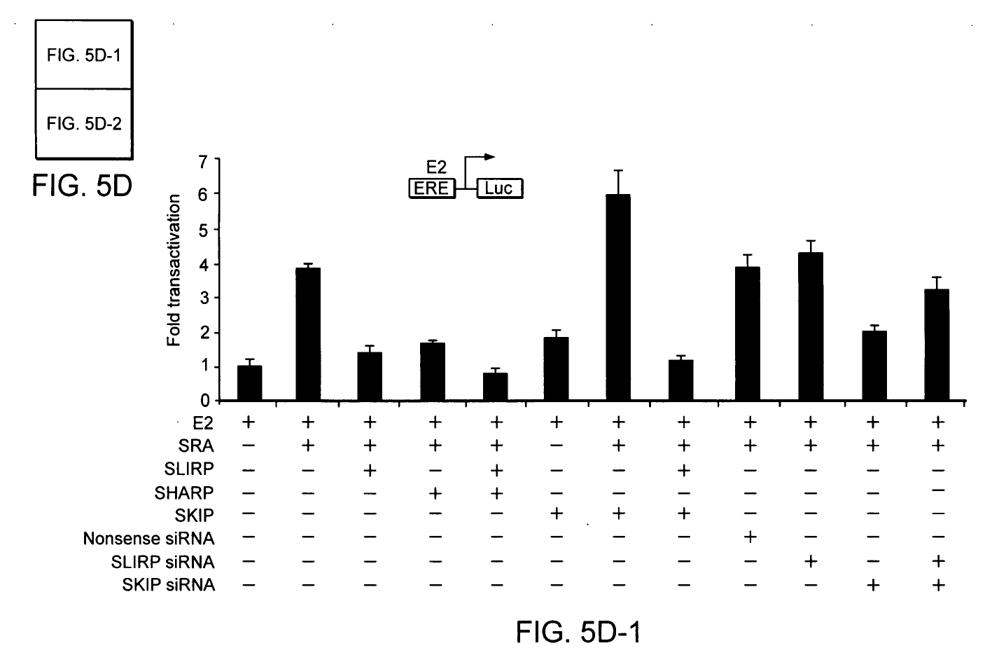

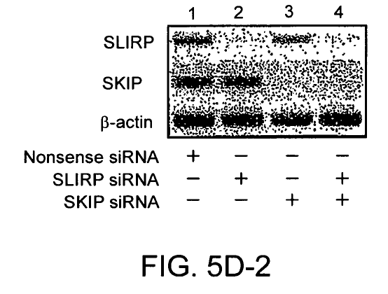

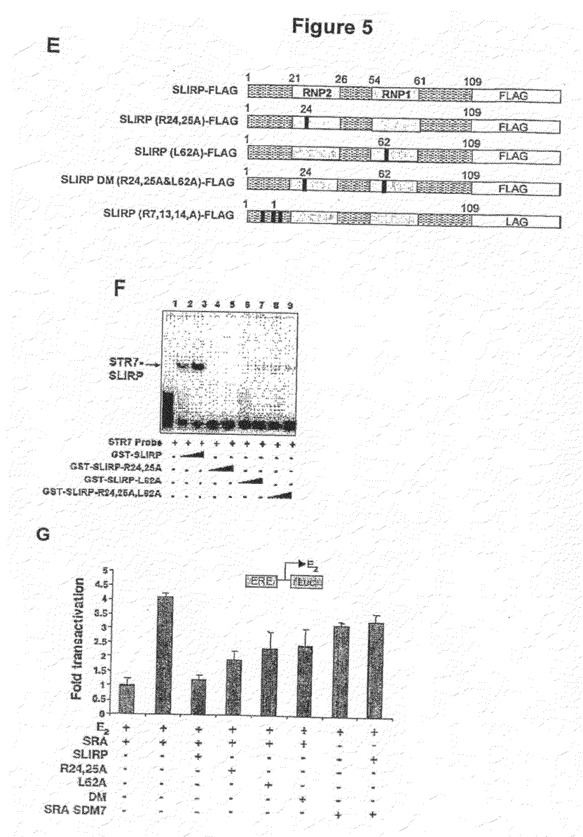

In transient transfection assays in HeLa cells using an E2-responsive reporter, we found that SRA coactivated reporter activity approximately 3-4 fold (FIG. 5A), as previously reported. When cotransfected with SRA, SLIRP repressed SRA-mediated coactivation (FIG. 5A). With increasing SLIRP concentrations, up to a 3-fold decrease of SRA-mediated coactivation was observed (FIG. 5A). These data define SLIRP as an estrogen receptor (ER) corepressor. Interestingly, addition of SLIRP to cells cotransfected with SRA and treated with estrogen antagonists, Tamoxifen (TAM) or ICI 182780, FasIodex, AstraZeneca) (ICI) further augmented repression of reporter activity (FIG. 5A, lanes 6-9).

We next investigated if the repression activity of SLIRP was evident with other NRs using a dexamethasone (Dex)-responsive glucocorticoid receptor (GR) reporter system. Cotransfection of SLIRP, with the reporter and SRA, into HeLa cells resulted in strong repression of GR-mediated transactivation (FIG. 5B), indicating SLIRP can modulate different NR signalling pathways. SLIRP also repressed hormone-responsive reporter activity of several other NRs in HeLa cells, including the androgen receptor (AR), thyroid hormone receptor (TR), and vitamin D receptor (VDR) (FIG. 5B). These data suggest that SLIRP has broad corepressive activity within the NR superfamily.

To complement our SLIRP overexpression studies, we investigated the effects of SLIRP siRNA on Dex-responsive reporter activity. In cells with reduced endogenous SLIRP expression, we found that there was a 10-fold increase in GRE-luc activity, further confirming that SLIRP acts as a NR corepressor (FIG. 5C).

### Example 6

### SLIRP Modulates SHARP- and SKIP-Coregulation of NR Activity

The high amino acid sequence homology between SHARP and SLIRP and their avid binding to SRA STR7 in vitro suggested that a functional interaction may exist in vivo. When cotransfected with SRA, SHARP repressed SRA-mediated coactivation of the E2-responsive reporter (FIG. 5D, lane 4) as previously reported (1). When SLIRP was cotransfected with SHARP and SRA, an additional 2-fold repression of SRA-mediated coactivation was observed (FIG. 5D, lane 5). Thus, SHARP and SLIRP appear to act in an additive fashion to augment repression of the E2-responsive reporter.

We investigated the effects of SKIP on SLIRP repression in further transfection studies. In the presence of transfected SKIP alone, reporter activity was increased ˜2-fold (FIG. 5D, lane 6), consistent with SKIP functioning as a coactivator of ER transactivation. In the presence of cotransfected SRA, an additive effect was observed with a total increase in activity of ˜6-fold (FIG. 5D, lane 7). When SLIRP was additionally cotransfected, reporter activity was reduced by more than 5-fold (FIG. 5D, lane 8). Thus, in the presence of SRA, SLIRP is a potent repressor of SKIP-mediated coactivation.

### Example 7

### SLIRP Function Requires Intact RRM Domains

To investigate the structural and functional significance of the RRM domain within SLIRP, we generated mutations within the RNP1 and RNP2 submotifs of the RRM domain (FIG. 5E) for analysis in REMSA and reporter assays. Based on binding predictions from other RRM-containing proteins, arginine 24 and 25 were mutated to alanines (R24, 25A) in the RNP2 submotif, and within the RNP1 domain leucine 62 was mutated to alanine (L62A). A double mutant (DM) containing both the R24, 25A and L62A substitutions was also prepared. In REMSA studies, each of the mutations markedly reduced binding to the SRA STR7 probe (FIG. 5F, lanes 4-9, and FIG. 8B). When we evaluated the mutants in transfection assays, we found that each mutant partially relieved the SLIRP-mediated repression (FIG. 5G, lanes 4, 5 & 6), indicating the requirement of an intact RRM domain for SLIRP to function as a repressor of E2-induced SRA coactivation.

To examine the functional specificity of the SLIRP-STR7 interaction in vivo, we utilized the SRA-SDM7 mutant of SRA used in REMSA studies above (19), in which the stem-loop structure is mutated, but preserved. We found this mutation decreased SRA-mediated coactivation to ˜70% of wild-type levels (FIG. 5G, lane 7). Furthermore, when we cotransfected SLIRP with SRA-SDM7, SLIRP was unable to function as a repressor (FIG. 5G, lane 8). This data suggested that a direct interaction between SLIRP and STR7 is critical for SLIRP's repressive activity.

### Example 8

### SLIRP is Recruited to Endogenous Promoters

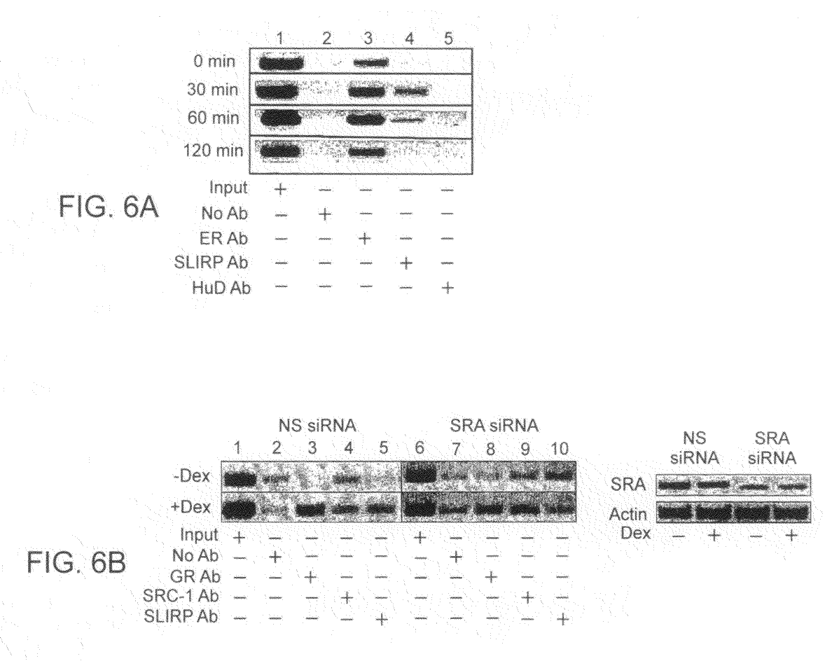

To determine if SLIRP was recruited to E2- and Dex-responsive promoters, we performed chromatin immunoprecipitation (ChIP) assays. We found that SLIRP was actively recruited to the E2-responsive pS2 promoter within 30 min (FIG. 6A). This association was reduced by 60 min, and had returned to undetectable levels by 120 min. ERα binding increased in response to ligand returning to basal levels within 120 min. In contrast HuD, another well-characterized RRM-containing RNA-binding protein, was not recruited to the DNA at all. This data confirmed that SLIRP, a protein that binds to and associates with SRA, can closely associate with the response element of an E2-regulated gene.

In an effort to better understand the mechanism by which SLIRP might mediate its effect at the transcriptional level, we performed ChIP assays in HeLa cells treated with SRA siRNA. Interestingly, these studies showed that in cells with reduced SRA expression, less SLIRP was recruited to the Dex-responsive metallothionein (MT) promoter (FIG. 6B, lane 10). This suggests that the presence of SRA is critical for recruiting SLIRP to the promoter and consequently mediating SLIRP's repressive effects.

To investigate possible interactions of SLIRP with other corepressors, we performed ChIP studies on MCF-7 breast cancer cells treated with Tamoxifen in the presence or absence of SLIRP siRNA. These studies showed that NCoR recruitment to the ERE was dependent upon SLIRP (FIG. 6C).

### Example 9

### SLIRP is Predominantly Mitochondrial

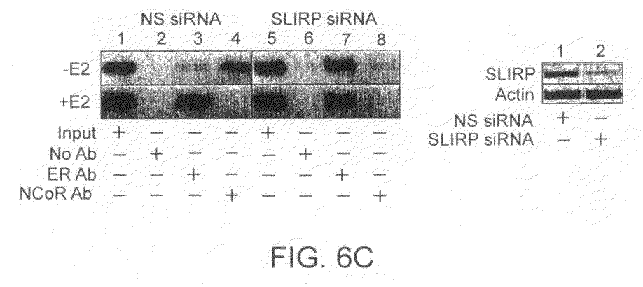

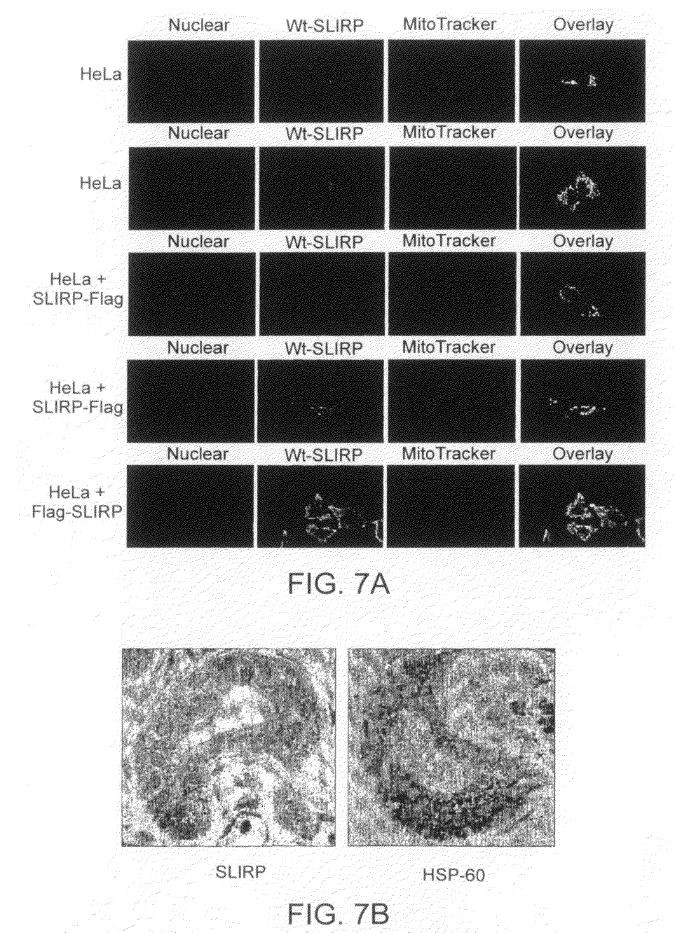

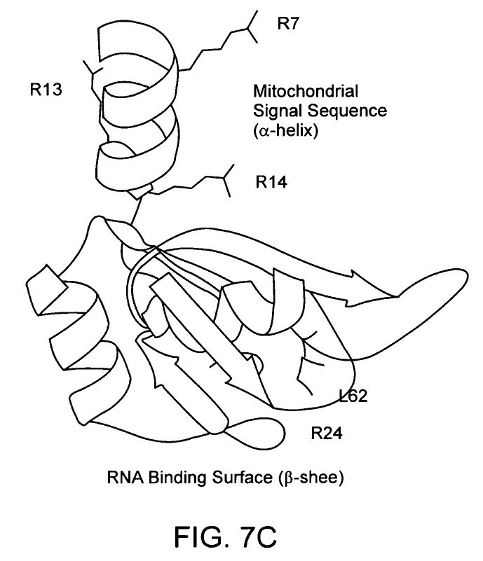

Based on our REMSA, transfection and ChIP data demonstrating that SLIRP is a NR transcriptional repressor, we envisaged SLIRP would be a predominantly nuclear protein. However, imaging studies using the SLIRP antibody revealed endogenous SLIRP to have a filamentous distribution confined predominantly to the cytoplasm. Using an organelle-specific counter stain we found that SLIRP localized to the mitochondria (FIG. 7A, top panel). A similar pattern was observed using antibody to HSP-60, a mitochondrial-specific protein (FIG. 7A, second row). In cells transfected with FLAG-tagged SLIRP, we found SLIRP colocalized with another mitochondrial-specific protein cytochrome C oxidase (FIG. 7A, third row). Sequence analysis revealed an N-terminal 26 amino acid domain within SLIRP that is highly predictive of an amphipathic α-helical mitochondrial targeting sequence and is conserved between the mouse, rat and human genomes (see FIG. 2D). This mitochondrial signal sequence is evident in the 3-D predicted structure of SLIRP, as an independent helix linked to the well conserved RRM structure (FIG. 7C). To evaluate the importance of the N-terminal signal sequence, we compared the intracellular localization of SLIRP-FLAG versus FLAG-SLIRP constructs. Interestingly, we found that SLIRP-FLAG localized to the mitochondria, whereas FLAG-SLIRP was pancellular (FIG. 7A, bottom two rows), consistent with the notion that the N-terminal mitochondrial signal sequence is a critical determinant for targeting SLIRP to the mitochondria.

To further investigate the mitochondrial location of endogenous SLIRP in human tissue, we examined primary human breast tissue with SLIRP and HSP-60 antibodies. A punctate cytoplasmic staining pattern, characteristic of mitochondria, was observed with both SLIRP and HSP-60 antibodies (FIG. 7B). In addition, we subfractionated human Jurkat cells to isolate the mitochondrial fraction, and used the HSP-60 antibody to confirm that SLIRP copurifies specifically with the mitochondrial fraction (data not shown). Taken together, these data confirm that SLIRP resides predominantly in the mitochondria, and that interference with the N-terminal signal sequence can substantially alter the intracellular distribution of the protein.

In order to evaluate the functional importance of the mitochondrial signal sequence, we generated a triple mutant of the first three arginines in SLIRP (R7, 13, 14, A) (see FIG. 6E). When cotransfected into HeLa cells with SRA, this SLIRP mutant had reduced ability to function as a repressor (FIG. 7D). This data suggested that the mitochondrial signal sequence is required for maintaining corepressor function, raising the possibility that SLIRP has a bifunctional capacity as a NR corepressor in the nucleus and mitochondria.

### Example 10

### SLIRP Regulates PPARδ Activity in Murine Muscle Cells

Materials/Methods

Conjugates containing 600 ng of PPRE-tkLUC reporter and either 65 ng of pSCT (23) plus 450 ng of pCMV-FLAG 5a (Sigma); 65 ng of pSCT plus 450 ng of pSLIRP-FLAG; 80 ng of pSCT-SRA (23) and 450 ng of pCMV-FLAG 5a; 80 ng of pSCT-SRA and 450 ng of pSLIRP-FLAG (24) expression vectors plus FuGene 6 (Roche) were prepared for transfection as per manufactures recommendations. These conjugates were then added to 1.5 mL volumes of C2C12 mouse muscle cells suspended at 70,000 cells per mL in complete medium RPMI (Invitrogen) plus 20% Serum Supreme (Biowhittaker) which were added to individual wells in 6 well plates. Cells plus transfection conjugates were maintained at 37° C. in a humidified, 5% CO2 environment for 12 hours before replacing of the media. Cells were cultured for a further 26 hours before addition of the PPARδ agonist GW501516 dissolved in DMSO to a final concentration of 1 uM. The same volume of DMSO was added to control cultures to a final concentration of 0.33%. 24 hours post ligand/vehicle treatment, media was removed and cells lysed in 150 uL of Passive Lysis Buffer (Promega) and the level of luciferase activity determined as described previously.

Results

SLIRP regulates PPARδ signalling activity (with a PPARE-Luc reporter) in the presence of a PPARδ-specific ligand (GW501516) in murine muscle cells (FIG. 9).

### Example 11

### SLIRP Predicts a Worse Survival in Human Breast Cancer

Kaplan Meier survival curves of SLIRP antibody reactivity in human breast cancer tissue microarrays. This represents data from over 500 primary breast cancer tissue samples, with a follow up period of 8.5 years. Tissue microarrays of primary human breast cancer tissue were immunostained using standard protocols. SLIRP polyclonal antibody was used at 1:4000 and biotinylated goat anti-rabbit and anti-mouse antibody (Chemicon IHC Select) followed by streptavidin-horseradish peroxidase as secondary and tertiary reagents. Sections visualized with diaminobenzidine (DAKO) followed by a light counterstain with haematoxylin.

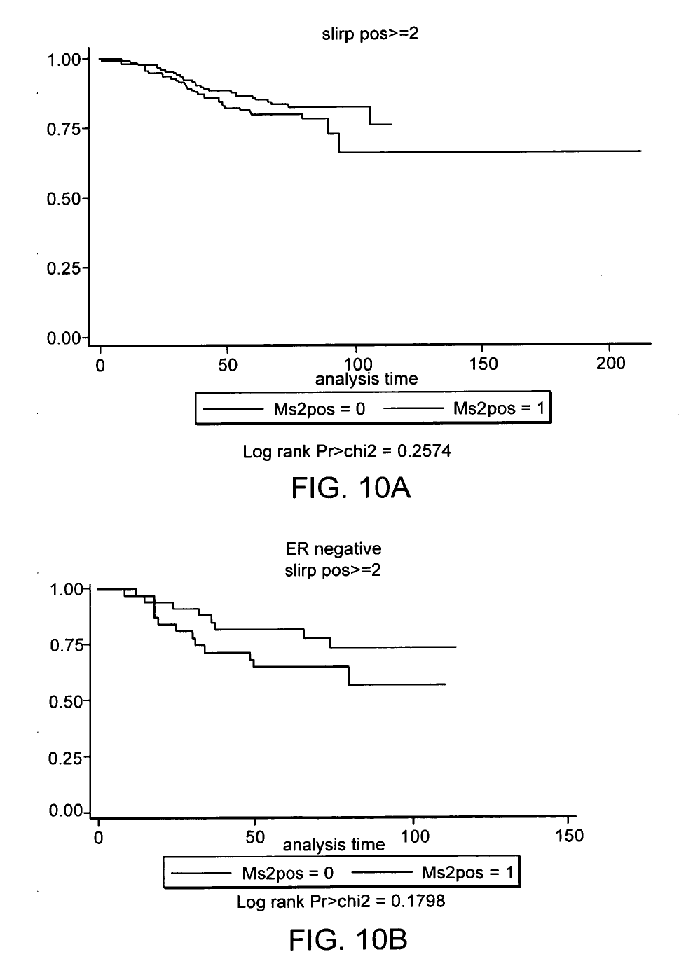

FIG. 10, panel A compares survival of patients with SLIRP positivity >2+ (on a scoring system of 0-3+, red line) versus those with SLIRP immunoreactivity less than or equal to 2 (blue line). FIG. 10, panel B represents survival with SLIRP staining of >2+ (red line) versus that of women with SLIRP less than or equal to 2 (blue line) specifically within the cohort of women with tumours that were estrogen receptor negative.

### Example 12

### SLIRP Downregulates Androgen Receptor Signalling in Prostate Cancer Cells

Materials/Methods

Human prostate cancer cells (22Rv1) were transfected in RPMI containing 5% stripped serum using FuGENE6 (Roche) with equal molar ratios of control empty and or cDNA expression plasmids plus: 0.6 μg/well PSA-Luc, 0.0625 μg/well pSCT or 0.08 μg/well pSCT-SRA, 0.049 μg/well pCMV-FLAG-7.1 or 0.05-0.5 μg/well FLAG-SLIRP. After transfection, cells were cultured for 8 h, prior to addition of DHT (10 nM). After 24 h, lysates assayed for luciferase activity relative to protein levels using Luciferase (Promega) and Protein Assays (BioRad) on a FluorStar Optima (BMG).

Results

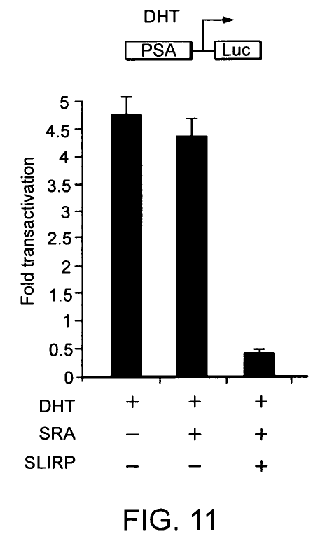

FIG. 11 shows that transfection of SLIRP into human prostate cancer cells (22Rv1 cells) that are androgen responsive, results in a reduction of androgen receptor mediated signalling, using an androgen-responsive PSA-Luc reporter. The PSA-Luc (prostate specific antigen-luciferase) reporter contains an androgen-responsive promoter, whose activity is inhibited ˜15-fold in the presence of excess SLIRP. This data indicates that SLIRP is a powerful repressor of androgen-mediated signalling in human prostate cancer cells.

### REFERENCES

1. Shi, Y., Downes, M., Xie, W., Kao, H.-Y., Ordentlich, P., Tsai, C.-C., Hon, M. & Evans, R. M. (2001) Genes & Development 15, 1140-1151.

2. Wantanabe, M., Yanagisawa, J., Kitagawa, H., Takeyama, K.-i., Ogawa, S., Arao, Y., Suzawa, M., Kobayashi, Y., Yano, T., Yoshikawa, H., Masuhiro, Y. & Kato, S. (2001) The EMBO Journal 20, 1342-1352.

3. SenGupta, D. J., Zhang, B., Kraemer, B., Pochart, P., Fields, S. & Wickens, M. (1996) Proc Natl Acad Sci USA 93, 8496-8501.

4. Byrne, J., Tomasetto, C., Garnier, J., Rouyer, N., Mattei, M., Bellocq, J., Rio, M. & Basset, P. (1995) Cancer Research 55, 2896-903.

5. Byrne, J., Nourse, C., Basset, P. & Gunning, P. (1998) Oncogene 16, 873-81.

6. Park, Y. W., Tan, S.-L. & Katze, M. G. (1999) Short Technical Reports 26, 1102-1106.

7. Burd, C., G & Dreyfuss, G. (1994) Science 265, 615-621.

8. Ghisolfi-Nieto, L., Joseph, G., Puvion-Dutilleul, F., Amalric, F. & Bouvet, P. (1996) Journal of Molecular Biology 260, 34-53

9. Bouvet, P., Jain, C., Belasco, J. & Amalric, A. F. (1997) EMBO J 16, 5235-46

10. Chenna, R., Sugawara, H., Tadashi, K., Lopez, R., Gibosn, T. J., Higgins, D. G. & Thompson, J. D. (2003) Nucleic Acids Research 31, 3497-500

11. Stothard, P. (2000) BioTechniques 28, 1102-04

12. Dowhan, D., Hong, E., Auboeuf, D., Dennis, A., Wilson, M., Berget, S. & O'Malley, B. W. (2005) Molecular Cell 17, 429-439.

13. Scheller, K. & Sekeris, C. (2003) Experimental Physiology 88, 129-40.

14. Tao, Y., Williams-Skipp, C. & Scheinman, R. I. (2001) Journal of Biological Chemistry 276, 2329-2332.

15. Zucker, M. (2003) Nucleic Acids Research 31, 3406-15.

16. Thomson, A. M., Rogers, J. T., Walker, C. E. & Leedman, P. J. (1999) BioTechniques 27, 1032-1042

17. Giles, K. M., Daly, J. M., Beveridge, D. J., Thomson, A. M., Voon, D. C., Furneaux, H. M., Jazayeri, J. A. & Leedman, P. J. (2003) Journal of Biological Chemistry 278, 2937-46

18. Munns, S. E., Lui, J. K., and Arthur, P. G. (2005). Mitochondrial hydrogen peroxide production alters oxygen consumption in an oxygen-concentration-dependent manner. Free Radic Biol Med 38, 1594-1603.

19. Lanz, R. B., Razani, B., Goldberg, A. D., and O'Malley, B. W. (2002). Distinct RNA motifs are important for coactivation of steroid hormone receptors by steroid receptor RNA activator (SRA) Proc Natl Acad Sci USA 99, 16081-16086.

20. Barry, J. B., Leong, G. M., Church, B., Issa, L. L. Eisman, J. A., and Gardiner, E. M. (2003). Interactions of SKIP/NCoA-62, TFIIB, and retinoid x receptor with vitamin D receptor helix H10 residues. Journal of Biological Chemistry 278, 8224-8228.

21. McDonald, P. N., Dowd, D. R., Zhang, C., and Gu, C. (2004) Emerging insights into the coactivator role of NCoA62/SKIP in vitamin D mediated transcription. Journal of Steroid Biochem Molec Biol 89-90, 179-186.

22. Bairoch, A., P, B., and K. H. (1997). The Prosite database, its status in 1997. Nucleic Acids Research 25, 217-221

23. Lanz, R. B., McKenna, N. J., Onate, S. A., Albrecht, U., Wong, J., Tsai, S. Y., Tsai, M.-J., and O'Malley, B. W. (1999). A steroid receptor coactivator, SRA, functions as an RNA and is present in an SRC-1 complex. Cell 97, 17-27.

24. Hatchell, E. M., Colley, S., Beveridge, D. J., Epis, M. R., Stuart, L. M., Giles, K. M., Redfern, A. D., Miles, L. E. C., Barker, A., MacDonald, L., Golding, J., M^{c}Culloch, R. M., Arthur, P., Lui, J. K. C., Wilce, J. A., Wilce, M. C. J., Lanz, R. B., O'Malley, B. W., Leedman, P. J. (2006) SLIRP, a small SRA-binding protein, is a nuclear receptor co-repressor. Molecular Cell 22, 657-668.

The claims defining the invention are as follows:

1. An isolated polypeptide comprising:
(i) SEQ ID No: 2;
(ii) amino acids 27 to 109 of SEQ ID No:2;
(iii) amino acids 22 to 109 of SEQ ID No:2;
(iv) amino acids 21 to 91 of SEQ ID No:2;
(v) amino acids 21-26 and/or 60-67 of SEQ ID No:2; or
(vi) a functional variant of any one of (i) to (v).

2. A fusion protein comprising a polypeptide of claim 1.

3. A method for identifying a functional variant of an isolated polypeptide according to any one of parts (i) to (v) of claim 1 comprising the steps of: (i) changing an amino acid residue of the isolated peptide to produce a variant and (ii) assessing the activity of the variant to identify functional variants.

4. A biologically active fragment of the polypeptide of SEQ ID No:2 comprising at least about 10, 20, 30, 50 or 100 amino acid residues.

5. A fragment according to claim 4 capable of modulating SRA regulated transactivation of a nuclear receptor.

6. A fragment according to claim 4 comprising an epitope-bearing portion of a polypeptide according to SEQ ID No:2.

7. Use of a polypeptide according to claim 1 for preparing a non-peptide mimetic thereof.

8. A non-peptide mimetic of a polypeptide of claim 1.

9. A selective binding agent of a polypeptide according to claim 1.

10. An antibody of a polypeptide according to claim 1.

11. A polyclonal antibody according to claim 10.

12. A monoclonal antibody according to claim 10.

13. A labelled antibody according to any one of claims 10 to 12.

14. A method for detecting a polypeptide according to claim 1 in a sample comprising the steps:
(i) providing an antibody of the invention;
(ii) contacting the sample with the antibody under conditions which allow for the formation of an antibody-antigen complex; and
(iii) determining whether antibody-antigen complex comprising the antibody is formed.

15. A method according to claim 14 wherein the sample is a tissue extract.

16. A method according to claim 15 wherein the tissue is selected from the group consisting of: brain, breast, ovary, lung, colon, pancreas, testes, skin, liver, muscle, prostate, bone tissue or a neoplastic growth derived from such a tissue.

17. An agent according to claim 9 or 10 bound to a solid support.

18. A method for identifying an agent that is capable of binding a polypeptide according to claim 1 comprising the steps of: (i) contacting an immobilized polypeptide according to claim 1 with a non-immobilized candidate agent and (ii) determining whether and/or to what extent the polypeptide and candidate agent bind to each other.

19. A method according to claim 18 wherein the polypeptide is immobilized on agarose beads.

20. A method for identifying an agent that is capable of modulating the binding of a polypeptide according to claim 1 to a ligand comprising the steps of: (i) contacting the polypeptide with the ligand in the presence and absence of the agent; and (ii) determining whether and/or to what extent the polypeptide binds the ligand.

21. A method for identifying an agent that is capable of modulating the activation of a nuclear receptor comprising the steps of: (i) activating the receptor in the presence and absence of the agent; and (ii) determining whether and/or to what extent the nuclear receptor is activated.

22. A method according to claim 20 or 21 wherein the agent is an antagonist.

23. A method according to claim 20 or 21 wherein the agent is an agonist.

24. A method according to claim 20 wherein the ligand is SRA.

25. A method according to claim 21 wherein the receptor is a Type I or Type II endocrine receptor.

26. An isolated polynucleotide encoding a polypeptide according to claim 1.

27. An isolated polynucleotide comprising SEQ ID No:1.

28. An isolated polynucleotide according to claim 26 or 27 comprising genomic DNA.

29. An isolated polynucleotide that selectively hybridizes to the polynucleotide of any one of claims 26.

30. An isolated polynucleotide according to claim 29 that comprises a nucleotide sequence 95% to 99% identical to a nucleotide sequence encoding the polypeptide having the complete amino acid sequence in SEQ ID NO: 2.

31. Use of a polynucleotide according to claim 27 for identifying a homologous polynucleotide.

32. A fusion polynucleotide comprising a polynucleotide according to any one of claims 26-30 and a marker sequence that encodes a peptide that facilitates purification of the expression product of the polynucleotide.

33. A vector comprising a polynucleotide according to any one of claims 26 to 30 or 32.

34. A host cell comprising a vector according to claim 33.

35. A pharmaceutical preparation comprising a polypeptide according to claim 1 and a physiologically acceptable carrier.

36. A method for treating a disease or disorder associated with an undesirable level of activation of a nuclear receptor, in a subject, the method comprising the step of administering the subject an effective amount of a polypeptide according to claim 1.

37. Use of a polypeptide according to claim 1 for preparing a medicament for treating a disease or disorder associated with an undesirable level of activation of a nuclear receptor.

38. A method according to claim 36 or a use according to claim 37 wherein the receptor belongs to the Type I endocrine NR subfamily.

39. A method or use according to claim 38 wherein the receptor is selected from the list consisting of: estrogen receptor α (ERα), estrogen receptor β (ERβ), progesterone receptor (PR), androgen receptor (AR), glucocorticoid receptor (GR), mineralocorticoid receptor (MR).

40. A method according to claim 36 or a use according to claim 37 wherein the receptor belongs to the Type II endocrine NR subfamily.

41. A method or use according to claim 40 wherein the receptor is selected from the list consisting of: retinoic acid receptor α (RARα), retinoic acid receptor β (RARβ), retinoic acid receptor γ (RARγ), thyroid hormone receptor α (TRα), thyroid hormone receptor β (TR β), vitamin D receptor (VDR), ecdysteroid receptor (EcR).

42. A method according to claim 36 or a use according to claim 37 wherein the receptor is selected from the list consisting of: retinoid X receptor α (RXRα), retinoid X receptor β (RXRβ), peroxisome proliferator activated receptor α (PPARα), peroxisome proliferator activated receptor β/δ (PPARβ/δ), peroxisome proliferator activated receptor γ (PPARγ), liver X receptor α (LXRα), liver X receptor β (LXRβ, farnesoid X receptor (FXR), pregnane X receptor (PXR), steroid and xenobiotic receptor (SXR) and constitutive andronstane receptor (CAR).

43. A method according to claim 36 or a use according to claim 37 wherein the disease or disorder is selected from the list consisting of: Cushings disease, hyperadrenocorticism, glucocorticoid excess related disorders, hypertension, ischaemic heart disease, dyslipidaemia (cholesterol and triglyceride abnormalities), apparent obesity, muscle wasting, thin skin, and metabolic aberrations such as diabetes, cancer such as prostate cancer, hirsuitism (or excessive hair growth), problems with libido and erectile dysfunction, osteoporosis, thyroid overactivity disorders, Graves' disease, toxic multinodular goitre and toxic nodules, rickets, osteoporosis, colonic carcinoma pituitary and adrenal disease that result in excess glucocorticoid production; disorders in which energy homeostasis is altered, including obesity, insulin resistance and diabetes mellitus; defects of fatty acid oxidation such as lipid storage myopathies; defects of the mitochondrial respiratory chain such as mitochondrial myopathies.

44. A method according to claim 36 or a use according to claim 37 wherein the disease or disorder is cancer.

45. A method or use according to claim 44 wherein the cancer is selected from the group consisting of: prostate, breast, ovary, skin, endometrium, kidney, lung, bone, liver, colon or cervical cancer.

46. Use of a polypeptide according to claim 1 or an agonist thereof for modulating SRA regulated transactivation of a nuclear receptor.

47. A method of modulating SRA regulated transactivation of a nuclear receptor comprising the step of contacting the SRA with an effective amount of a polypeptide according to claim 1 or an agonist thereof.

48. Use of a polypeptide according to claim 1 or an agonist thereof in combination with SHARP, SRC-1 or SKIP for modulating SRA regulated transactivation of a nuclear receptor.

49. A method of modulating SRA regulated transactivation of a nuclear receptor comprising the step of contacting the SRA with an effective amount of a polypeptide according to claim 1 or an agonist thereof and SHARP, SRC-1 or SKIP.

50. Use of an antagonist of the polypeptide according to claim 1 for modulating SRA regulated transactivation of a nuclear receptor.

51. A method of enhancing SRA mediated activation of a nuclear receptor comprising the step of contacting the SRA with an effective amount of a polypeptide according to claim 1 or an antagonist thereof.

52. A method for performing a diagnosis on a patient comprising:
(i) determining the amount of a polypeptide according to SEQ ID No:2 in a sample, taken from the patient;
(ii) comparing the amount determined in step (i) to the concentration range 25 of the polypeptide known to be present in normal subjects; and
(iii) diagnosing whether the patient has a disorder based on the comparison in step (ii).

53. A method for prognostic evaluation of a patient comprising:
(i) determining the concentration of a polypeptide according to SEQ ID No:2 in a biological sample, taken from the patient;
(ii) comparing the level determined in step (i) to the concentration range of the polypeptide known to be present in normal subjects; and
(iii) evaluating the prognosis of said patient based on the comparison in step (ii).

54. A method for determining a therapeutic intervention in a patient with a disorder comprising:
(iv) determining the concentration of a polypeptide according to SEQ ID No:2 in a biological sample, taken from the patient;
(v) comparing the level determined in step (i) to the concentration range of the polypeptide known to be present in normal subjects; and
(vi) evaluating the therapeutic intervention for the disorder based on the comparison in step (ii).

55. Use of a polynucleotide according to SEQ ID No:1 or a fragment thereof as a marker for cancer.

56. A method of genotyping a subject comprising the step of contacting a sample from said subject with a probe capable of hybridizing to a polynucleotide encoding a polypeptide according to claim 1 and detecting the hybridization products.

57. A method according to claim 57 wherein the probe is capable of hybridizing to an allelic variant of SEQ ID No:1 that results in the subject producing a sub-optimal amount of a polypeptide according to SEQ ID No:2.

58. A solid substrate having immobilized thereon at least one polynucleotide according to claim 27.

59. A transgenic animal comprising a polynucleotide according to claim 27.


SEQUENCE LISTING (25 sequences)

SEQ ID NO: 1 - DNA, 399 bp, Homo sapiens
  CDS (37)..(363)
ggccattatggccggggaaggtgctttagtctgaagatggcggcctcagcagcgcgaggtgctgcggcgctgcgtagaagtatcaatcagccggttgcttttgtgagaagaattccttggactgcggcgtcgagtcagctgaaagaacactttgcacagttcggccatgtcagaaggtgcattttaccttttgacaaggagactggctttcacagaggtttgggttgggttcagttttcttcagaagaaggacttcggaatgcactacaacaggaaaatcatattatagatggagtaaaggtccaggttcacactagaaggccaaaacttccgcaaacatctgatgatgaaaagaaagatttttgagactgcagcctattaataaagttaacataactg

SEQ ID NO: 2 - protein, 109 aa, Homo sapiens
MAASAARGAAALRRSINQPVAFVRRIPWTAASSQLKEHFAQFGHVRRCILPFDKETGFHRGLGWVQFSSEEGLRNALQQENHIIDGVKVQVHTRRPKLPQTSDDEKKDF

SEQ ID NO: 3 - RNA, 89 nt, Artificial Sequence
  Description of Artificial Sequence Synthetic probe
aggaagcagguaugugaugacaucagccgacgccuggcacugcugcaggaacagugggcuggaggaaaguugucaauaccuguaaagaa

SEQ ID NO: 4 - protein, 73 aa, Homo sapiens
GIKVQNLPVRSTDTSLKDGLFHEFKKFGKVTSVQIHGTSEERYGLVFFRQQEDQEKALTASKGKLFFGMQIEV

SEQ ID NO: 5 - protein, 77 aa, Homo sapiens
TRTLFIGNLEKTTTYHDLRNIFQRFGEIVDIDIKKVNGVPQYAFLQYCDIASVCKAIKKMDGEYLGNNRLKLGFGKS

SEQ ID NO: 6 - protein, 73 aa, Homo sapiens
NCVWLDGLSSNVSDQYLTRHFCRYGPVVKVVFDRLKGMALVLYNEIEYAQAAVKETKGRKIGGNKIKVDFANR

SEQ ID NO: 7 - protein, 71 aa, Homo sapiens
TLFVKGLSEDTTEETLKESFDGSVRARIVTDRETGSSKGFGFVDFNSEEDAKAAKEAMEDGEIDGNKVTLD

SEQ ID NO: 8 - protein, 112 aa, Mus musculus
MAASAIKGLSALRSSTGRPIAFVRKIPWTAAASELREHFAQFGHVRRCTVHFDKETGFHRGMGWVQFSSQEELQNALQQEHHIIDGVKIHVQAQRAKALHGAQTSDEERFLR

SEQ ID NO: 9 - protein, 111 aa, Rattus norvegicus
MAASAVKGLTALRSRTGRPIAFIRKIPWTAASSELREHFAQFGHVRRCTVPFDKETGFHRGMGWVQFSSQEELQNALQEEHHIIDGVEIHVQPQKPKVLHGAQTSDEEKDF

SEQ ID NO: 10 - protein, 6 aa, Artificial Sequence
  Description of Artificial Sequence Synthetic 6xHis tag
HHHHHH

SEQ ID NO: 11 - protein, 4 aa, Artificial Sequence
  Description of Artificial Sequence Synthetic peptide
GGGG

SEQ ID NO: 12 - DNA, 24 bp, Artificial Sequence
  Description of Artificial Sequence Synthetic primer
cgcggatccgcggcctcagcagca

SEQ ID NO: 13 - DNA, 25 bp, Artificial Sequence
  Description of Artificial Sequence Synthetic primer
gcgcggatcctaggctgcagtctca

SEQ ID NO: 14 - DNA, 89 bp, Artificial Sequence
  Description of Artificial Sequence Synthetic primer
aggaggcaggtatgtgatgacatcagccgacgcctggcactgctgcaggaacagtgggctggaggaaagttgtcaatacctgtaaagaa

SEQ ID NO: 15 - DNA, 89 bp, Artificial Sequence
  Description of Artificial Sequence Synthetic primer
ttctttacaggtattgacaactttcctccagcccactgttcctgcagcagtgccaggcgtcggctgatgtcatcacatacctgcttcct

SEQ ID NO: 16 - DNA, 21 bp, Artificial Sequence
  Description of Artificial Sequence Synthetic primer
tgatgacatcagccgacgcct

SEQ ID NO: 17 - DNA, 21 bp, Artificial Sequence
  Description of Artificial Sequence Synthetic primer
gctgcagatttctcttcattg

SEQ ID NO: 18 - DNA, 20 bp, Artificial Sequence
  Description of Artificial Sequence Synthetic primer
gcgctgcgtagaagtatcaa

SEQ ID NO: 19 - DNA, 20 bp, Artificial Sequence
  Description of Artificial Sequence Synthetic primer
tcgattccgaagtccttctt

SEQ ID NO: 20 - DNA, 20 bp, Artificial Sequence
  Description of Artificial Sequence Synthetic primer
gccaacacagtgctgtctgg

SEQ ID NO: 21 - DNA, 20 bp, Artificial Sequence
  Description of Artificial Sequence Synthetic primer
tactcctgcttgctgatcca

SEQ ID NO: 22 - DNA, 29 bp, Artificial Sequence
  Description of Artificial Sequence Synthetic primer
ggccatctctcactatgaatcacttctgc

SEQ ID NO: 23 - DNA, 26 bp, Artificial Sequence
  Description of Artificial Sequence Synthetic primer
ggcaggctctgtttgcttaaagagcg

SEQ ID NO: 24 - DNA, 20 bp, Artificial Sequence
  Description of Artificial Sequence Synthetic primer
actcgtcccggctctttcta

SEQ ID NO: 25 - DNA, 19 bp, Artificial Sequence
  Description of Artificial Sequence Synthetic primer
aggagcagttgggatccat